(19) United States
(12) Patent Application Publication
(10) Pub. No.: US 2003/0215916 A1
(43) Pub. Date: Nov. 20, 2003

(54) Novel imidazoline receptor homologs

(76) Inventors: John N. Feder, Belle Mead, NJ (US); Gene G. Kinney, Collegeville, PA (US); Gabriel Mintier, Hightstown, NJ (US); Chandra S. Ramanathan, Wallingford, CT (US); David K. Bol, Gaithersburg, MD (US); Rolf-Peter Ryseck, Ewing, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/395,812

(22) Filed: Mar. 21, 2003

Related U.S. Application Data
(63) Continuation-in-part of application No. 09/932,145, filed on Aug. 17, 2001.
(60) Provisional application No. 60/261,779, filed on Jan. 16, 2001.
(60) Provisional application No. 60/226,411, filed on Aug. 18, 2000.

(51) Int. Cl. C12Q 1/68; G01N 33/53; G01N 33/567; C07H 21/04; C07K 14/705; C12P 21/02; C12N 5/06

(52) U.S. Cl. 435/69.1; 435/320.1; 435/325; 530/350; 536/23.5; 435/6; 435/7.2; 530/388.22

(57) ABSTRACT

Novel imidazoline receptor homologs, designated imidazoline receptor related protein 1 (IMRRP1), imidazoline receptor related protein 1b (IMRRP1b), and derivatives thereof are described. Pharmaceutical compositions comprising at least one IMRRP1, IMRRP1b, or a functional portion thereof, are provided as are methods for producing IMRRP1, IMRRP1b or a functional portion thereof. In addition, nucleic acid sequences encoding polypeptides, oligonucleotides, fragments, portions or antisense molecules thereof, and expression vectors and host cells comprising polynucleotides that encode IMRRP1 or IMRRP1b are provided. The novel association of IMRRP1 and/or IMRRP1b to modulating the NFkB pathway and the p21 cell cycle checkpoint, and uses thereof are also provided.

This application is a continuation-in-part application of non-provisional application U.S. Ser. No. 09/932,145, filed Aug. 17, 2001; which claims priority to provisional application U.S. Serial No. 60/261,779 filed Jan. 16, 2001, and to provisional application, U.S. Serial No. 60/226,411 filed Aug. 18, 2000, under 35 U.S.C. 119(e). The entire teachings of the referenced applications are incorporated herein by reference.

### FIELD OF THE INVENTION

Novel imidazoline receptor homologs, designated imidazoline receptor related protein 1 (IMRRP1), imidazoline receptor related protein 1b (IMRRP1b) and derivatives thereof are described. Pharmaceutical compositions comprising at least one IMRRP1, IMRRP1b or a functional portion thereof are provided as are methods for producing IMRRP1, IMRRP1b or a functional portion thereof. In addition, nucleic acid sequences encoding polypeptides, oligonucleotides, fragments, portions or antisense molecules thereof, and expression vectors and host cells comprising polynucleotides that encode IMRRP1 or IMRRP1b are provided. The use of the nucleic acid sequences, polypeptide, peptide and antibodies for diagnosis and treatment of disorders or diseases associated with aberrant regulation of blood pressure, induction of feeding, stimulation of firing of locus coeruleus neurons, and stimulation of insulin release, as well as the aberrant induction of the expression of glial fibrillary acidic protein independent of the action of alpha-2 adrenoceptors, dysphoric premenstrual syndrome, neurodepolynucleotiderative disorders such as Alzheimer's disease, opiate addiction, monoamine turnover and therefore nociception, aging, mood and stroke, salivary disorders and developmental disorders is also described.

### BACKGROUND OF THE INVENTION

Imidazoline receptor (IMR) subtypes bind clonidine and imidazoline (Escriba et al., 1995). These compounds mediate the regulation of blood pressure, induction of feeding, stimulation of firing of locus coeruleus neurons, and stimulation of insulin release, as well as the induction of the expression of glial fibrillary acidic protein independent of the action of alpha-2 adrenoceptors. These receptors are pharmacologically important target for drugs that can mediate the aforementioned physiological conditions (Farsang and Kapocsi, 1999).

Non-adrenoceptor sites predominantly labeled by clonidine or para-amino clonidine are termed I₁-sites whereas those non-adrenoceptor sites predominantly labeled by idazoxan are termed I₂-sites. Imidazoline sites which are distinct from either I₁- or I₂ sites are termed I₃-sites. An example is an imidazoline receptor in the pancreas reported to enhance insulin secretion. Chan et al. (1993) Eur. J. Pharmocol. 230 375; Chan et al. (1994) Br. J. Pharmocol. 112 1065. The receptor is efaroxan sensitive and it a target for the treatment of type II diabetes. The site is also sensitive to agmatine, an insulin secretagogue, and to crude preparation of clonidine displacing substance (CDS). I₂ sites may also be involved in the modulation of smooth muscle proliferation.

Endogenous ligands of the imidazoline receptors are harmane, tryptamine and agmatine. There are also numerous compounds which are selective for either I1-sites, e.g., clonidine, benazoline and rilmenidine, or I₂-sites, e.g. RS-45041-190, 2-BFI, BU 224, and BU 239. Many of these compounds are commercially available, for example, from Tocris Cookson, Inc., USA.

I₁-site selective drugs are promising for the treatment of hypertension, I₃-site selective drugs are promising for the treatment of diabetes, and I₂-site selective drugs affect monoamine turnover and therefore I₂ receptor ligands can affect a wide range of brain functions such as nociception, ageing, mood and stroke.

Characterization of the immidazoline receptor polypeptides of the present invention led to the determination that they are involved in the modulation of the p21 G1/S-phase cell cycle check point protein, either directly or indirectly.

Critical transitions through the cell cycle are highly regulated by distinct protein kinase complexes, each composed of a cyclin regulatory and a cyclin-dependent kinase (cdk) catalytic subunit (for review see Draetta, Curr. Opin. Cell Biol. 6, 842-846 (1994)). These proteins regulate the cell's progression through the stages of the cell cycle and are, in turn, regulated by numerous proteins, including p53, p21, p16, and cdc25. Downstream targets of cyclin-cdk complexes include pRb and E2F. The cell cycle often is dysregulated in neoplasia due to alterations either in oncopolynucleotides that indirectly affect the cell cycle, or in tumor suppressor polynucleotides or oncopolynucleotides that directly impact cell cycle regulation, such as pRb, p53, p16, cyclin D1, or mdm-2 (for review see Schafer, Vet Pathol 1998 35, 461-478 (1998)).

P21, also known as CDNK1A (cyclin-dependent kinase inhibitor 1A), or CIP1 inhibits mainly the activity of cyclin CDK2 or CDK4 complexes. Therefore, p21 primarily blocks cell cycle progression at the G1 stage of the cell cycle. The expression of p21 is tightly controlled by the tumor suppressor protein p53, through which this protein mediates the cell cycle G1 phase arrest in response to a variety of stress stimuli. In addition, p21 protein interacts with the DNA polymerase accessory factor PCNA (proliferating cell nuclear antigen), and plays a regulatory role in S phase DNA replication and DNA damage repair.

After DNA damage, many cells appear to enter a sustained arrest in the G2 phase of the cell cycle. Bunz et al. (Science 282, 1497-1501 (1998)) demonstrated that this arrest could be sustained only when p53 was present in the cell and capable of transcriptionally activating the cyclin-dependent kinase inhibitor p21. After disruption of either the p53 or the p21 polynucleotide, gamma-radiated cells progressed into mitosis and exhibited a G2 DNA content only because of a failure of cytokinesis. Thus, p53 and p21 appear to be essential for maintaining the G2 cell cycle checkpoint in human cells.

Due to the connection between the transcriptional activity of p53 and p21 RNA expression, the readout of p21 RNA can be used to determine the effect of drugs or other insults (radiation, antisense for a specific polynucleotide, dominant negative expression) on a given cell system which contains wild type p53. Specifically, if a polynucleotide is removed using antisense products and this has an effect on the p53 activity, p21 will be upregulated and can serve therefore as an indirect marker for an influence on the cell cycle regulatory pathways and induction of cell cycle arrest.

In addition to cancer regulation of cell cycle activity has a role in numerous other systems. For example, hematopoietic stem cells are relative quiescent, while after receiving the required stimulus they undergo dramatic proliferation and inexorably move toward terminal differentiation. This is partly regulated by the presence of p21. Using p21 knockout mice Cheng et al. (Science 287, 1804-1808 (2000)) demonstrated its critical biologic importance in protecting the stem cell compartment. In the absence of p21, hematopoietic stem cell proliferation and absolute number were increased under normal homeostatic conditions. Exposing the animals to cell cycle-specific myelotoxic injury resulted in premature death due to hematopoietic cell depletion. Further, self-renewal of primitive cells was impaired in serially transplanted bone marrow from p21 −/− mice, leading to hematopoietic failure. Therefore it was concluded that p21 is the molecular switch governing the entry of stem cells into the cell cycle, and in its absence, increased cell cycling leads to stem cell exhaustion. Under conditions of stress, restricted cell cycling is crucial to prevent premature stem cell depletion and hematopoietic death. Therefore, polynucleotides involved in the downregulation of p21 expression could have a stimulatory effect and therefore be useful for the exploration of stem cell technologies.

Characterization of the immidazoline receptor polypeptides of the present invention led to the determination that it is involved in the NFkB pathway through modulation of the IkB protein, either directly or indirectly.

The fate of a cell in multicellular organisms often requires choosing between life and death. This process of cell suicide, known as programmed cell death or apoptosis, occurs during a number of events in an organisms life cycle, such as for example, in development of an embryo, during the course of an immunological response, or in the demise of cancerous cells after drug treatment, among others. The final outcome of cell survival versus apoptosis is dependent on the balance of two counteracting events, the onset and speed of caspase cascade activation (essentially a protease chain reaction), and the delivery of antiapoptotic factors which block the caspase activity (Aggarwal B. B. Biochem. Pharmacol. 60, 1033-1039, (2000); Thoruberry, N. A. and Lazebnik, Y. Science 281, 1312-1316, (1998)).

The production of antiapoptotic proteins is controlled by the transcriptional factor complex NF-kB. For example, exposure of cells to the protein tumor necrosis factor (TNF) can signal both cell death and survival, an event playing a major role in the regulation of immunological and inflammatory responses (Ghosh, S., May, M. J., Kopp, E. B. Annu. Rev. Immunol. 16, 225-260, (1998); Silverman, N. and Maniatis, T., Genes & Dev. 15, 2321-2342, (2001); Baud, V. and Karin, M., Trends Cell Biol. 11, 372-377, (2001)). The anti-apoptotic activity of NF-kB is also crucial to oncopolynucleotidesis and to chemo- and radio-resistance in cancer (Baldwin, A. S., J. Clin. Inves. 107, 241-246, (2001)).

Nuclear Factor-kB (NF-kB), is composed of dimeric complexes of p50 (NF-kB1) or p52 (NF-kB2) usually associated with members of the Rel family (p65, c-Rel, Rel B) which have potent transactivation domains. Different combinations of NF-kB/Rel proteins bind distinct kB sites to regulate the transcription of different polynucleotides. Early work involving NF-kB suggested its expression was limited to specific cell types, particularly in stimulating the transcription of polynucleotides encoding kappa immunoglobulins in B lymphocytes. However, it has been discovered that NF-kB is, in fact, present and inducible in many, if not all, cell types and that it acts as an intracellular messenger capable of playing a broad role in polynucleotide regulation as a mediator of inducible signal transduction. Specifically, it has been demonstrated that NF-kB plays a central role in regulation of intercellular signals in many cell types. For example, NF-kB has been shown to positively regulate the human beta-interferon (beta-IFN) polynucleotide in many, if not all, cell types. Moreover, NF-kB has also been shown to serve the important function of acting as an intracellular transducer of external influences.

The transcription factor NF-kB is sequestered in an inactive form in the cytoplasm as a complex with its inhibitor, IkB, the most prominent member of this class being IkBa. A number of factors are known to serve the role of stimulators of NF-kB activity, such as, for example, TNF. After TNF exposure, the inhibitor is phosphorylated and proteolytically removed, releasing NF-kB into the nucleus and allowing its transcriptional activity. Numerous polynucleotides are upregulated by this transcription factor, among them IkBa. The newly synthezised IkBa protein inhibits NF-kB, effectively shutting down further transcriptional activation of its downstream effectors. However, as mentioned above, the IkBa protein may only inhibit NF-kB in the absence of IkBa stimuli, such as TNF stimulation, for example. Other agents that are known to stimulate NF-kB release, and thus NF-kB activity, are bacterial lipopolysaccharide, extracellular polypeptides, chemical agents, such as phorbol esters, which stimulate intracellular phosphokinases, inflammatory cytokines, IL-1, oxidative and fluid mechanical stresses, and Ionizing Radiation (Basu, S., Rosenzweig, K, R., Youmell, M., Price, B, D, Biochem, Biophys, Res, Commun., 247(l):79-83, (1998)). Therefore, as a polynucleotideral rule, the stronger the insulting stimulus, the stronger the resulting NF-kB activation, and the higher the level of IkBa transcription. As a consequence, measuring the level of IkBa RNA can be used as a marker for antiapoptotic events, and indirectly, for the onset and strength of pro-apoptotic events.

The upregulation of IkBa due to the downregulation of the immidazoline receptors places these GPCR proteins into a signalling pathway potentially involved in apoptotic events. This gives the opportunity to regulate downstream events via the activity of the protein of the immidazoline receptors with antisense polynucleotides, polypeptides or low molecular chemicals with the potential of achieving a therapeutic effect in cancer, autoimmune diseases. In addition to cancer and immunological disorders, NF-kB has significant roles in other diseases (Baldwin, A. S., J. Clin Invest. 107, :3-6 (2001)). NF-kB is a key factor in the pathophysiology of ischemia-reperfusion injury and heart failure (Valen, G., Yan. Z Q, Hansson, G K, J. Am. Coll. Cardiol. 38, 307-14 (2001)). Furthermore, NF-kB has been found to be activated in experimental renal disease (Guijarro C, Egido J., Kidney Int. 59, 415-425 (2001)).

Antisense inhibition of the immidazoline receptor protein provokes a response in A549 cells that indicates the regulatory pathways controlling p21 and IkB-alpha levels are affected. See example IX and X herein. This implicates the imidazoline receptor in pathways important for maintenance of the proliferative state and progression through the cell cycle. As stated above, there are numerous pathways that could have either indirect or direct affects on the transcriptional levels of p21 and IkB-alpha. Importantly, a major part of the pathways implicated involve the regulation of protein activity through phosphorylation. In as much as the immidazoline receptor is a phosphatase enzyme, it is readily conceivable that dephosphorylation of proteins, the counter activity to the kinases in the signal transduction cascades, contributes to the signals determining cell cycle regulation and proliferation, including regulating p21 and IkB-alpha levels. Additionally, the complexity of the interactions between proteins in the pathways described also allow for affects on the pathway eliciting compensatory responses. That is, inhibition of one pathway affecting p21 and IkB-alpha activity could provoke a more potent response and signal from another pathway of the same end, resulting in upregulation of p21 and IkB-alpha. Thus, the effect of inhibition of the immidazoline receptor resulting in slight increases in the immidazoline receptor levels could indicate that one pathway important to cancer is effected in a way to implicate the immidazoline receptor as a potential target for pharmacologic inhibition for cancer treatment, yet a parallel pathway in the context of the experiment would replace the immidazoline receptor and propagate dysregulation of p21 and IkB-alpha.

Characterization of the imidazoline receptor polypeptides of the present invention led to the determination that they are direct or indirect members of the leucine-rich repeat superfamily. LRR regions typically contain 20-29 amino acids with asparagine and leucine in conserved positions. Proteins with this motif participate in molecular recognition processes and cellular processes that include signal transduction, cellular adhesion tissue organization, hormone binding and RNA processing. These LRR proteins have been linked to human pathologies such as breast cancer and gliomas.

### SUMMARY OF THE INVENTION

The present invention relates to novel imidazoline receptor homologs, hereinafter designated imidazoline receptor related protein 1 (IMRRP1) (SEQ ID NO:3), imidazoline receptor related protein 1b (IMRRP1b) (SEQ ID NO:4) and derivatives thereof.

Accordingly, the invention relates to a substantially purified IMRRP1 having the amino acid sequence of FIGS. 1A-C (SEQ ID NO:3), or functional portion thereof, and a substantially purified variant of IMRRP1, referred to as IMRRP1b, having the amino acid sequence of FIGS. 2A-D (SEQ ID NO:4).

The present invention further provides a substantially purified soluble IMRRP1 polypeptide. In a particular aspect, the soluble IMRRP1 comprises the amino acid sequence of FIGS. 1A-C (SEQ ID NO:3). The present invention further provides a substantially purified soluble IMRRP1b polypeptide. In a particular aspect, the soluble IMRRP1 comprises the amino acid sequence of FIGS. 2A-D (SEQ ID NO:4).

The present invention provides pharmaceutical compositions comprising one IMRRP1 polypeptides, fragments, or a functional portion thereof.

The present invention provides pharmaceutical compositions comprising one IMRRP1b polypeptides, fragments, or a functional portion thereof.

The present invention also provides methods for producing IMRRP1, IMRRP1b, fragments or functional portion(s) thereof.

One aspect of the invention relates to isolated and substantially purified polynucleotides that encode IMRRP1 or IMRRP1b. In a particular aspect, the polynucleotide comprises the nucleotide sequence of FIGS. 1A-C (SEQ ID NO:1). In another aspect of the invention, the polynucleotide comprises the nucleotide sequence which encodes IMRRP1.

In another aspect, the polynucleotide comprises the nucleotide sequence of FIGS. 2A-D (SEQ ID NO:2). In another aspect of the invention, the polynucleotide comprises the nucleotide sequence which encodes the IMRRP1 variant, IMRRP1b (SEQ ID NO:2).

The invention also relates to a polynucleotide sequence comprising the complement of the sequence provided in FIGS. 1A-C (SEQ ID NO:1), FIGS. 2A-D (SEQ ID NO:2), or variants thereof.

In addition, the invention features polynucleotide sequences which hybridize under stringent conditions to a polynucleotide sequence provided in FIGS. 1A-C (SEQ ID NO:1), FIGS. 2A-D (SEQ ID NO:2), or variants thereof.

The invention further relates to nucleic acid sequences encoding polypeptides, oligonucleotides, fragments, portions or antisense molecules thereof, and expression vectors and host cells comprising polynucleotides that encode the IMRRP1 or IMRRP1b polypeptide.

It is another object of the present invention to provide methods for producing polynucleotide sequences encoding an imidazoline receptor.

Another aspect of the invention is antibodies which bind specifically to an imidazoline receptor or epitope thereof, for use as therapeutics and diagnostic agents.

Another aspect of the invention is an agonist, antagonist, or inverse agonist of the IMRRP1 and/or IMRRP1b polypeptide.

The present invention provides methods for screening for agonists, antagonists and inverse agonists of the imidazoline receptors.

It is another object of the present invention to use the nucleic acid sequences, polypeptide, peptide and antibodies for diagnosis of disorders or diseases associated with aberrant regulation of blood pressure, induction of feeding, stimulation of firing of locus coeruleus neurons, and stimulation of insulin release, as well as the aberrant induction of the expression of glial fibrillary acidic protein independent of the action of alpha-2 adrenoceptors, dysphoric premenstrual syndrome, neurodepolynucleotiderative disorders such as Alzheimer's disease, opiate addiction, monoamine turnover and therefore nociception, aging, mood and stroke, salivary disorders and developmental disorder, including aberrant epithelial or stromal cell growth, in addition to other proliferating disorders including angiopolynucleotidesis, and apoptosis, and/or cancers.

The present invention provides methods of preventing or treating disorders associated with aberrant regulation of blood pressure, induction of feeding, stimulation of firing of locus coeruleus neurons, and stimulation of insulin release, as well as methods of preventing or treating disorders associated with the aberrant induction of the expression of glial fibrillary acidic protein independent of the action of alpha-2 adrenoceptors, dysphoric premenstrual syndrome, neurodepolynucleotiderative disorders such as Alzheimer's disease, opiate addiction, monoamine turnover and therefore nociception, ageing, mood and stroke, salivary disorders and developmental disorders.

The invention further relates to a method for preventing, treating, or ameliorating a medical condition with the polypeptides provided as SEQ ID NO:3 in addition to, its encoding nucleic acid, or a modulator thereof, wherein the medical condition is a disorder associated with aberrant p21 expression or activity.

The invention further relates to a method for preventing, treating, or ameliorating a medical condition with the polypeptides provided as and SEQ ID NO:4, in addition to, its encoding nucleic acid, or a modulator thereof, wherein the medical condition is a disorder associated with aberrant p21 expression or activity.

The invention further relates to a method for preventing, treating, or ameliorating a medical condition with the polypeptides provided as SEQ ID NO:3, in addition to, its encoding nucleic acid, or a modulator thereof, wherein the medical condition is a cell cycle defect, disorders related to aberrant phosphorylation, disorders related to aberrant signal transduction, proliferating disorders, and/or cancers.

The invention further relates to a method for preventing, treating, or ameliorating a medical condition with the polypeptides provided as SEQ ID NO:4, in addition to, its encoding nucleic acid, or a modulator thereof, wherein the medical condition is a cell cycle defect, disorders related to aberrant phosphorylation, disorders related to aberrant signal transduction, proliferating disorders, and/or cancers.

The invention further relates to a method for preventing, treating, or ameliorating a medical condition with the polypeptides provided as SEQ ID NO:3, in addition to, its encoding nucleic acid, or a modulator thereof, wherein the medical condition is a disorder associated with aberrant cell cycle regulation.

The invention further relates to a method for preventing, treating, or ameliorating a medical condition with the polypeptides provided as SEQ ID NO:4, in addition to, its encoding nucleic acid, or a modulator thereof, wherein the medical condition is a disorder associated with aberrant cell cycle regulation.

The invention further relates to a method of increasing, or alternatively decreasing, the number of cells in the G1 phase of the cell cycle comprising the step of administering an antagonist, or alternatively an agonist, of the IMRRP1 and/or IMRRP1b polypepide.

The invention further relates to a method of increasing, or alternatively decreasing, the number of cells in the G2 phase of the cell cycle comprising the step of administering an antagonist, or alternatively an agonist, of the IMRRP1 and/or IMRRP1b polypepide.

The invention further relates to a method of decreasing, or alternatively increasing, the number of cells that progress to the S phase of the cell cycle comprising the step of administering an antagonist, or alternatively an agonist, of the IMRRP1 and/or IMRRP1b polypepide.

The invention further relates to a method of increasing, or alternatively decreasing, the number of cells that progress to the M phase of the cell cycle comprising the step of administering an antagonist, or alternatively an agonist, of the IMRRP1 and/or IMRRP1b polypepide.

The invention further relates to a method of inducing, or alternatively inhibiting, cells into G1 and/or G2 phase arrest comprising the step of administering an antagonist, or alternatively an agonist, of the IMRRP1 and/or IMRRP1b polypepide.

The invention also relates to an antisense compound 8 to 30 nucleotides in length that specifically hybridizes to a nucleic acid molecule encoding the human IMRRP1 and/or IMRRP1b polypeptide of the present invention, wherein said antisense compound inhibits the expression of the human the IMRRP1 and/or IMRRP 1b polypepide.

The invention further relates to a method of inhibiting the expression of the human IMRRP1 or IMRRP1b polypeptide of the present invention in human cells or tissues comprising contacting said cells or tissues in vitro, or in vivo, with an antisense compound of the present invention so that expression of the the IMRRP1 and/or IMRRP1b polypepide is inhibited.

The invention further relates to a method of increasing, or alternatively decreasing, the expression of p21 in human cells or tissues comprising contacting said cells or tissues in vitro, or in vivo, with an antisense compound that specifically hybridizes to a nucleic acid molecule encoding the human the IMRRP1 and/or IMRRP1b polypepide of the present invention so that expression of the the IMRRP1 and/or IMRRP1b polypepide is inhibited.

The present invention is also directed to a method of identifying a compound that modulates the biological activity of the IMRRP1 and/or IMRRP1b polypepide, comprising the steps of, (a) combining a candidate modulator compound with the IMRRP1 and/or IMRRP1b polypepide in the presence of an antisense molecule that antagonizes the activity of the IMRRP1 and/or IMRRP1b polypeptide selected from the group consisting of SEQ ID NO:14, 15, 16, 17, and/or 18, and (b) identifying candidate compounds that reverse the antagonizing effect of the peptide.

The present invention is also directed to a method of identifying a compound that modulates the biological activity of the IMRRP1 and/or IMRRP1b polypepide, comprising the steps of, (a) combining a candidate modulator compound with the IMRRP1 and/or IMRRP1b polypepide in the presence of a small molecule that antagonizes the activity of the the IMRRP1 and/or IMRRP1b polypepide selected from the group consisting of SEQ ID NO:14, 15, 16, 17, and/or 18, and (b) identifying candidate compounds that reverse the antagonizing effect of the peptide.

The present invention is also directed to a method of identifying a compound that modulates the biological activity of the IMRRP1 and/or IMRRP1b polypepide, comprising the steps of, (a) combining a candidate modulator compound with the IMRRP1 and/or IMRRP1b polypepide in the presence of a small molecule that agonizes the activity of the IMRRP1 and/or IMRRP1b polypepide selected from the group consisting of SEQ ID NO:3 and/or 4, and (b) identifying candidate compounds that reverse the agonizing effect of the peptide.

The invention further relates to a method for preventing, treating, or ameliorating a medical condition with the polypeptide provided as SEQ ID NO:3, in addition to, its encoding nucleic acid, or a modulator thereof, wherein the medical condition is uterine cancer or related proliferative condition of the epithelium.

The invention further relates to a method for preventing, treating, or ameliorating a medical condition with the polypeptide provided as SEQ ID NO:4, in addition to, its encoding nucleic acid, or a modulator thereof, wherein the medical condition is uterine cancer or related proliferative condition of the epithelium.

The invention further relates to a method of diagnosing a pathological condition or a susceptibility to a pathological condition in a subject comprising the steps of (a) determining the presence or amount of expression of the polypeptide of SEQ ID NO:3 in a biological sample; (b) and diagnosing a pathological condition or a susceptibility to a pathological condition based on the presence or amount of expression of the polypeptide relative to a control, wherein said condition is a member of the group consisting of breast, testicular, ovarian, uterine, or prostate cancer, colon cancer, pancreatic cystadenoma, uterine epithelial tumors, and cancers of the gastrointestinal tract.

The invention further relates to a method of diagnosing a pathological condition or a susceptibility to a pathological condition in a subject comprising the steps of (a) determining the presence or amount of expression of the polypeptide of SEQ ID NO:4 in a biological sample; (b) and diagnosing a pathological condition or a susceptibility to a pathological condition based on the presence or amount of expression of the polypeptide relative to a control, wherein said condition is a member of the group consisting of breast, testicular, ovarian, uterine, or prostate cancer, colon cancer, pancreatic cystadenoma, uterine epithelial tumors, and cancers of the gastrointestinal tract.

The present invention provides kits for screening and diagnosis of disorders associated with aberrant IMRRP1 and/or IMRRP1b polypepide.

### BRIEF DESCRIPTION OF THE FIGURES

These and other objects, features and many of the attendant advantages of the invention will be better understood upon a reading of the detailed description of the invention when considered in connection with the accompanying drawings herein:

FIGS. 1A-C show the polynucleotide sequence from Clone No. FL1-18, referred to as IMRRP1 (SEQ ID NO:1), and the encoded polypeptide sequence (SEQ ID NO:3). Clone No. FL1-18 was deposited as ATCC Deposit No. PTA-2671 on Nov. 15, 2000 at the American Type Culture Collection, Patent Depository, 10801 University Boulevard, Manassas, Va. 20110-2209.

FIGS. 2A-D show the polynucleotide sequence from Clone No. FL1-18 splice variant (SEQ ID NO:2), referred to as IMRRP1b, and the encoded polypeptide sequence (SEQ ID NO:4).

FIG. 3 shows an alignment between the IMRRP1 polypeptide of the present invention with the human imidazoline receptor (Genbank Accession No:NP_{—}009115; SEQ ID NO:7).

FIG. 4 shows an alignment between the polynucleotide sequence of a clone that encodes the IMRRP1 polypeptide of the present invention, FL1-18, to a partial clone, Incyte 2499870. Top strand, FL1-18; bottom strand, Incyte 2499870.

FIGS. 5A and B shows a local sequence alignment between the encoded polypeptide sequence of the FL1-18 splice variant polynucleotide to the Drosophila melanogaster CG9044 (Genbank Accession No. gi|24582055; SEQ ID NO:11), and human imidazoline receptor (Genbank Accession No:NP_{—}009115; SEQ ID NO:7).

FIGS. 6A-D show a global sequence alignment between the IMRRP1 polypeptide (SEQ ID NO:3) and IMRR1b polypeptide (SEQ ID NO:4), to the LkB1-interacting protein 1 (Genbank Accession No., SEQ ID NO:33), the human KMOT1a (International Publication No. WO 20/24750; SEQ ID NO:34), the Drosophila melanogaster CG9044 (Genbank Accession No. gi|24582055; SEQ ID NO:11), and human imidazoline receptor (Genbank Accession No:NP_{—}009115; SEQ ID NO:7).

FIG. 7 shows the expression profile of IMRRP1. As shown, the IMRRP1 polypeptide is expressed predominately in testis; significantly in placenta, bone marrow, lymph node, and to a lesser extent in other tissues shown.

FIG. 8 shows an expanded expression profile of IMRRP1. As shown, the IMRRP1 polypeptide is expressed predominately in testis; and significantly in fetal brain and salivary gland.

FIG. 9 shows the expression profile of IMRRP1 in a panel of cancer cell lines. The IMRRP1 encoding mRNA is expressed in many tumor cell lines with a high degree of differential expression between the various cell lines (up to 10 fold). Specifically, IMRRP1 transcripts were predominately expressed in lung and breast cancer cell lines. The latter results suggest an association between the IMRRP1 protein and aberrant cell growth in these tissues.

FIG. 10 shows an expanded expression profile of the novel imidazoline receptor homolog polypeptide, IMRRP1, in normal tissues. As shown, the IMRRP1 polypeptide was predominately expressed in testis, significantly in fallopian tube, lymph gland, lung, brain, and to a lesser extent in other tissues.

FIG. 11 shows an expanded expression profile of IMRRP1 of the present invention. The figure illustrates the relative expression level of IMRRP1 amongst various mRNA tissue sources isolated from normal and tumor tissues. As shown, the IMRRP1 polypeptide was differentially expressed in expressed in breast, testicular, and colon cancers compared to each respective normal tissue.

### DESCRIPTION OF THE INVENTION

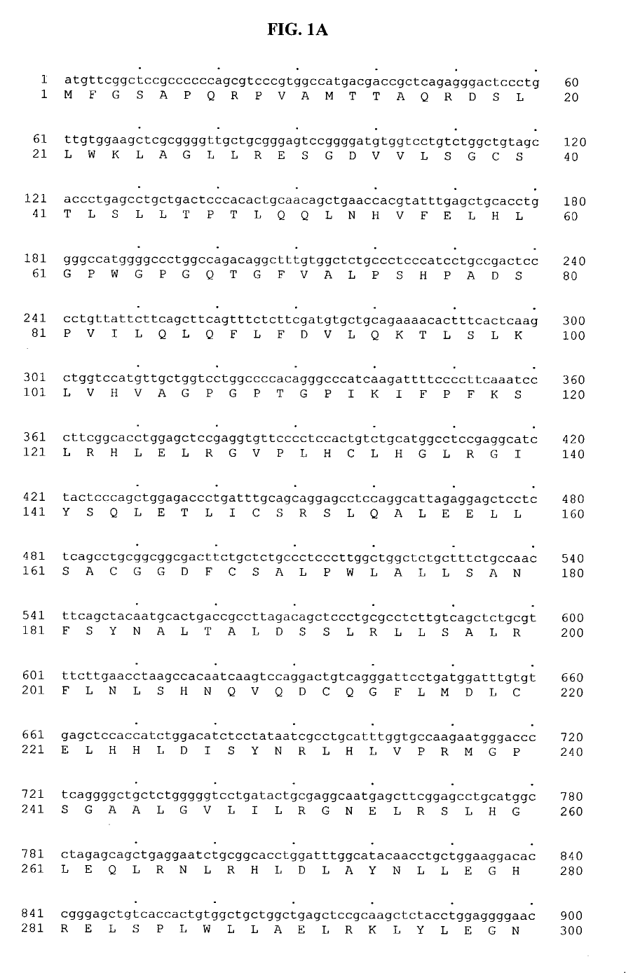

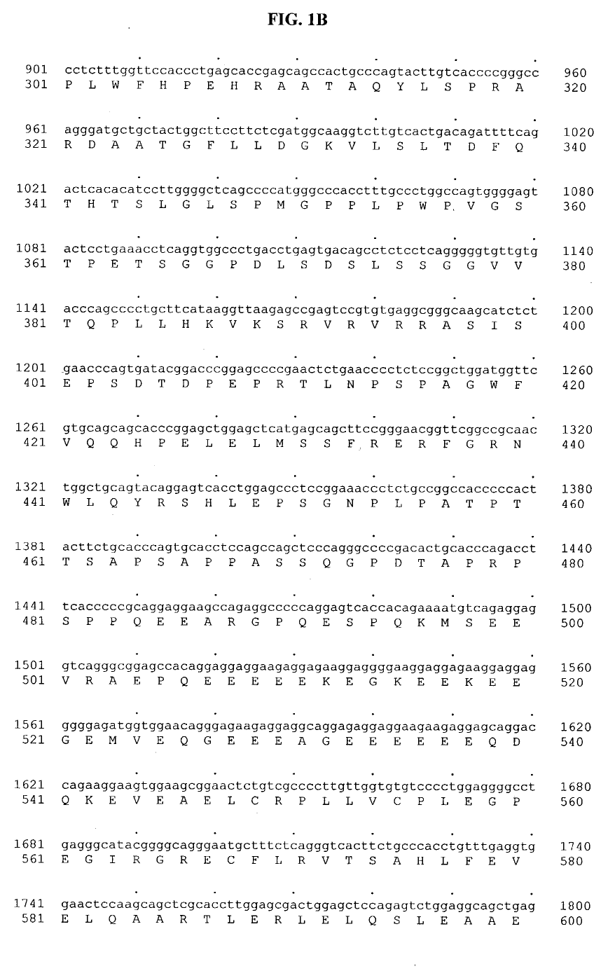

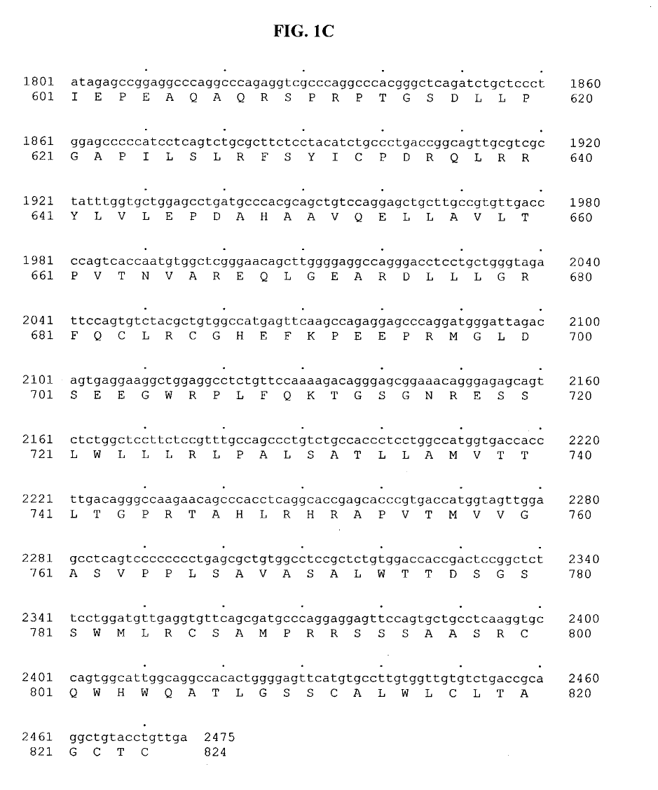

The present invention provides isolated nucleic acid molecules, that comprise, or alternatively consist of, a polynucleotide encoding the IMRRP1 protein having the amino acid sequence shown in FIGS. 1A-C (SEQ ID NO:3), or the amino acid sequence encoded by the cDNA clone, IMRRP1 deposited as ATCC Deposit Number PTA-2671 on Nov. 15, 2000.

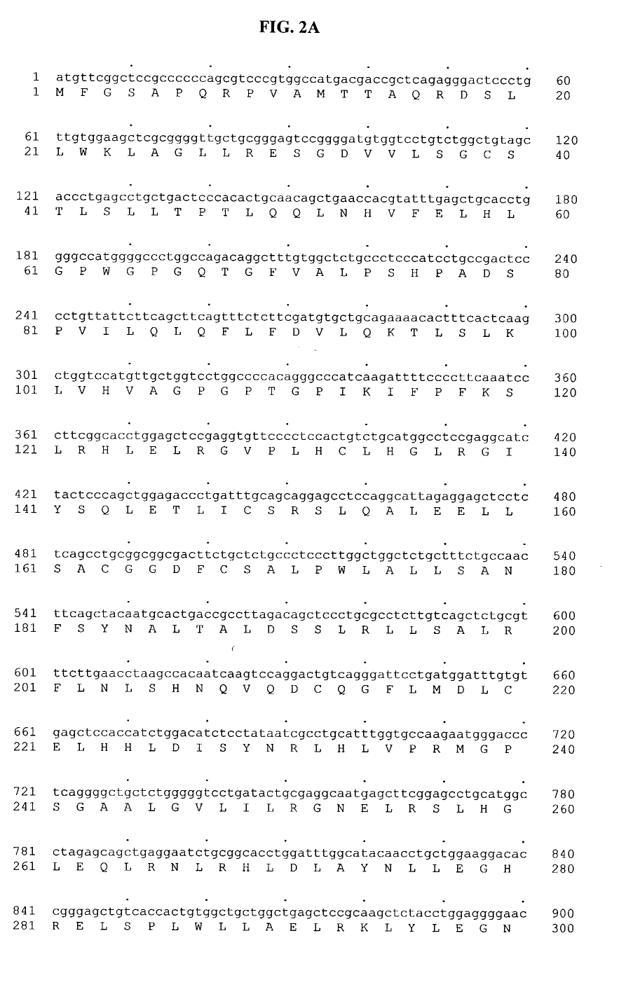

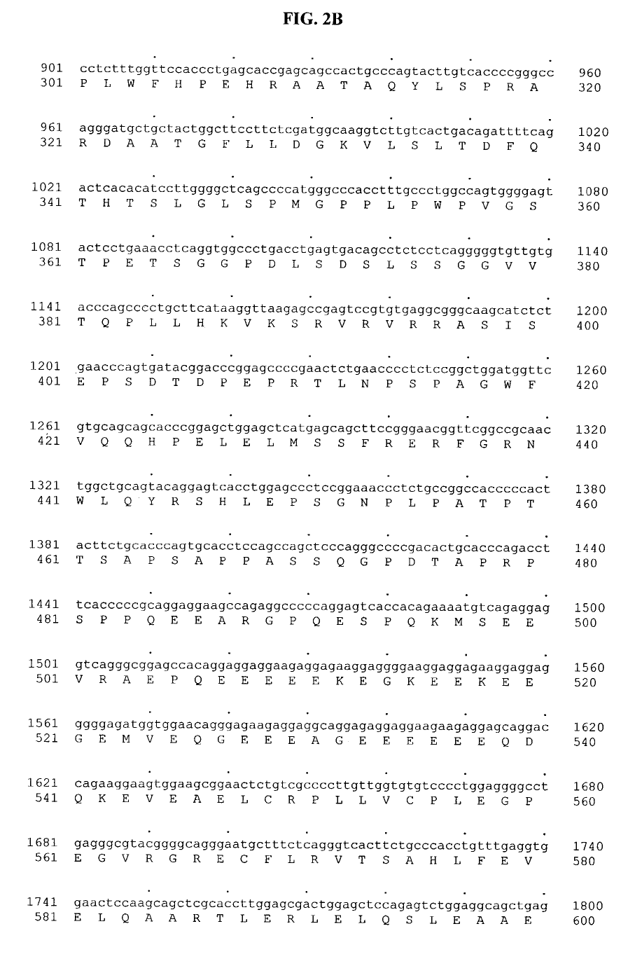

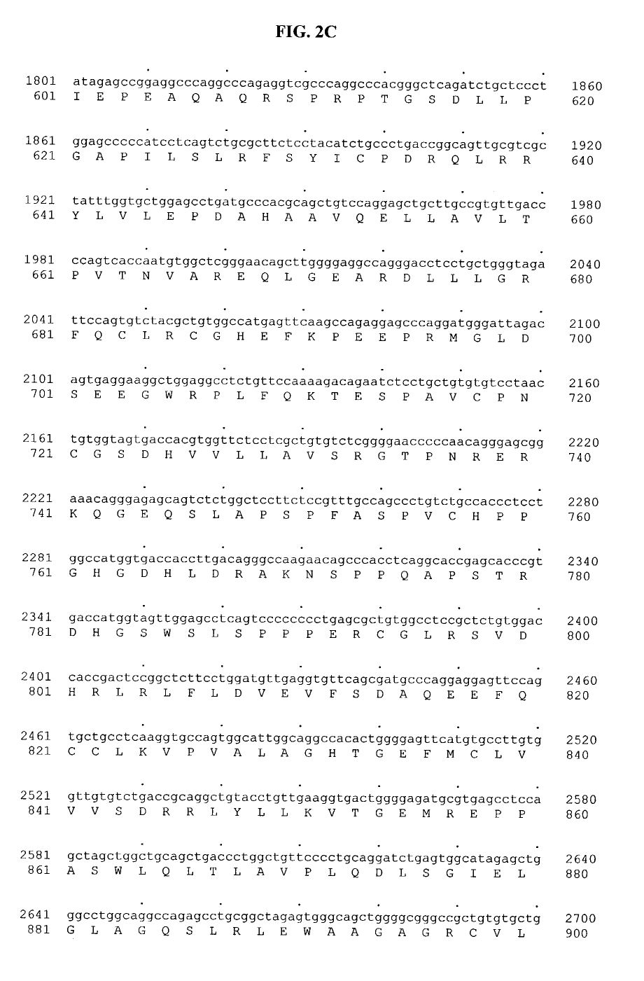

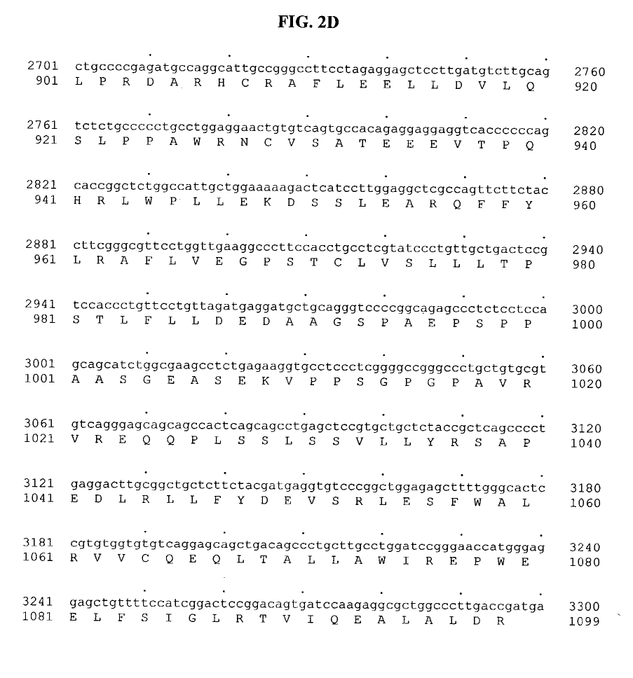

The present invention provides isolated nucleic acid molecules, that comprise, or alternatively consist of, a polynucleotide encoding the IMRRP1b protein having the amino acid sequence shown in FIGS. 2A-D (SEQ ID NO:4).

All references to “IMRRP1” shall be construed to apply to IMRRP1, IMRRP1b, and vise versa, unless otherwise specified herein.”

“Nucleic acid sequence”, as used herein, refers to an oligonucleotide, nucleotide, or polynucleotide, and fragments or portions thereof, and to DNA or RNA of genomic or synthetic origin which may be single- or double-stranded, and represent the sense or antisense strand. Similarly, “amino acid sequence” as used herein refers to an oligopeptide, peptide, polypeptide, or protein sequence, and fragments or portions thereof, and to naturally occurring or synthetic molecules.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

“Peptide nucleic acid”, as used herein, refers to a molecule which comprises an oligomer to which an amino acid residue, such as lysine, and an amino group have been added. These small molecules, also designated anti-polynucleotide agents, stop transcript elongation by binding to their complementary strand of nucleic acid (Nielsen, P. E. et al (1993) Anticancer Drug Des., 8:53-63).

IMRRP1 and IMRRP1b, as used herein, refer to the amino acid sequences of substantially purified imidazoline receptor related proteins obtained from any species, particularly mammalian, including bovine, ovine, porcine, murine, equine, and preferably human, from any source whether natural, synthetic, semi-synthetic, or recombinant.

“Consensus”, as used herein, refers to a nucleic acid sequence which has been resequenced to resolve uncalled bases, or which has been extended using XL-PCR (Perkin Elmer, Norwalk, Conn.) in the 5′ and/or the 3′ direction and resequenced, or which as been assembled from the overlapping sequences of more than one Incyte clone or publically available clone using the GELVIEW Fragment Assembly system (GCG, Madison, Wis.), or which has been both extended and assembled.

A “variant” of IMRRP1 or IMRRP1b, as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions, or both. Guidance in determining which amino acid residues may be substituted, inserted, or deleted without abolishing biological or immunological activity may be found using computer programs well known in the art, for example, DNASTAR software.

A “deletion”, as used herein, refers to a change in either amino acid or nucleotide sequence in which one or more amino acid or nucleotide residues, respectively, are absent.

An “insertion” or “addition”, as used herein, refers to a change in an amino acid or nucleotide sequence resulting in the addition of one or more amino acid or nucleotide residues, respectively, as compared to the naturally occurring molecule.

A “substitution”, as used herein, refers to the replacement of one or more amino acids or nucleotides by different amino acids or nucleotides, respectively.

The term “biologically active”, as used herein, refers to a protein having structural, regulatory, or biochemical functions of a naturally occurring molecule. Likewise, “immunologically active” refers to the capability of the natural, recombinant, or synthetic imidazoline receptor, or any oligopeptide thereof, to induce a specific immune response in appropriate animals or cells and to bind with specific antibodies.

The term “agonist”, as used herein, refers to a molecule which when bound to IMRRP1 or IMRRP1b, increases the amount of, or prolongs the duration of, the activity of IMRRP1 or IMRRP1b. Agonists may include proteins, nucleic acids, carbohydrates, organic molecules or any other molecules which bind to IMRRP1 or IMRRP1b.

The term “antagonist”, as used herein, refers to a molecule which, when bound to IMRRP1 or IMRRP1b, decreases the biological or immunological activity of IMRRP1 or IMRRP1b. Antagonists and inhibitors may include proteins, nucleic acids, carbohydrates, organic molecules or any other molecules which bind to IMRRP1 or IMRRP1b.

The term “mimetic”, as used herein, refers to a molecule, the structure of which is developed from knowledge of the structure of IMRRP1 or IMRRP1b or portions thereof and, as such, is able to effect some or all of the actions of IMRRP1 or IMRRP1b.

The term “derivative”, as used herein, refers to the chemical modification of a nucleic acid encoding IMRRP1 or IMRRP1b or the encoded IMRRP1 or IMRRP1b. Illustrative of such modifications would be replacement of hydrogen by an alkyl, acyl, or amino group. A nucleic acid derivative would encode a polypeptide which retains essential biological characteristics of the natural molecule.

The term “substantially purified”, as used herein, refers to nucleic or amino acid sequences that are removed from their natural environment, isolated or separated, and are at least 60% free, preferably 75% free, and most preferably 90% or greater free from other components with which they are naturally associated.

“Amplification”, as used herein, refers to the production of additional copies of a nucleic acid sequence and is polynucleotiderally carried out using polymerase chain reaction (PCR) technologies well known in the art (Dieffenbach, D. W. and G. S. Dveksler (1995), PCR Primer, a Laboratory Manual, Cold Spring Harbor Press, Plainview, N.Y.).

The term “hybridization”, as used herein, refers to any process by which a strand of nucleic acid binds with a complementary strand through base pairing.

The term “hybridization complex”, as used herein, refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cₒt or Rₒt analysis) or between one nucleic acid sequence present in solution and another nucleic acid sequence immobilized on a solid support (e.g., membranes, filters, chips, pins or glass slides to which cells have been fixed in situ hybridization).

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands.

The term “homology”, as used herein, refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence.

As known in the art, numerous equivalent conditions may be employed to comprise either low or high stringency conditions. Factors such as the length and nature (DNA, RNA, base composition) of the sequence, nature of the target (DNA, RNA, base composition, presence in solution or immobilization, etc.), and the concentration of the salts and other components (e.g., the presence or absence of formamide, dextran sulfate and/or polyethylene glycol) are considered and the hybridization solution may be varied to polynucleotiderate conditions of either low or high stringency different from, but equivalent to, the above listed conditions.

The term “stringent conditions”, as used herein, is the “stringency” which occurs within a range from about Tm−5° C. (5° C. below the melting temperature TM of the probe) to about 20° C. to 25° C. below Tm. As will be understood by those of skill in the art, the stringency of hybridization may be altered in order to identify or detect identical or related polynucleotide sequences.

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the polynucleotide(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be polynucleotiderated. The designation “negative” is sometimes used in reference to the antisense strand, and “positive” is sometimes used in reference to the sense strand.

The term “portion”, as used herein, with regard to a protein (as in “a portion of a given protein”) refers to fragments of that protein. The fragments may range in size from four amino acid residues to the entire amino acid sequence minus one amino acid. Thus, a protein “comprising at least a portion of the amino acid sequence of SEQ ID NO:3 or 4” encompasses the full-length human IMRRP1 or IMRRP1b and fragments thereof.

“Transformation”, as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and partial bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “antigenic determinant”, as used herein, refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “specific binding” or “specifically binding”, as used herein, in reference to the interaction of an antibody and a protein or peptide, mean that the interaction is dependent upon the presence of a particular structure (i.e., the antigenic determinant or epitope) on the protein; in other words, the antibody is recognizing and binding to a specific protein structure rather than to proteins in polynucleotideral. For example, if an antibody is specific for epitope “A”, the presence of a protein containing epitope A (or free, unlabeled A) in a reaction containing labeled “A” and the antibody will reduce the amount of labeled A bound to the antibody.

The term “sample”, as used herein, is used in its broadest sense. A biological sample suspected of containing nucleic acid encoding IMRRP1 or IMRRP1b or fragments thereof may comprise a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells or a tissue, and the like.

The term “correlates with expression of a polynucleotide”, as used herein, indicates that the detection of the presence of ribonucleic acid that is similar to SEQ ID NOS: 1 or 2 by northern analysis is indicative of the presence of mRNA encoding IMRRP1 and IMRRP1b in a sample and thereby correlates with expression of the transcript from the polynucleotide encoding the protein.

“Alterations” in the polynucleotide of SEQ ID NOS: 1 and 2 as used herein, comprise any alteration in the sequence of polynucleotides encoding IMRRP1 and IMRRP1b including deletions, insertions, and point mutations that may be detected using hybridization assays. Included within this definition is the detection of alterations to the genomic DNA sequence which encodes IMRRP1 or IMRRP1b (e.g., by alterations in the pattern of restriction fragment length polymorphisms capable of hybridizing to SEQ ID NOS: 1 or 2), the inability of a selected fragment of SEQ ID NOS: 1 or 2 to hybridize to a sample of genomic DNA (e.g., using allele-specific oligonucleotide probes), and improper or unexpected hybridization, such as hybridization to a locus other than the normal chromomsomal locus for the polynucleotide sequence encoding IMRRP1 or IMRRP1b (e.g., using fluorescent in situ hybridization (FISH) to metaphase chromosome spreads).

As used herein, the term “antibody” refers to intact molecules as well as fragments thereof, such as Fa, F(ab′)₂, Fv, chimeric antibody, single chain antibody which are capable of binding the epitopic determinant. Antibodies that bind IMRRP1 or IMRRP1b polypeptides can be prepared using intact polypeptides or fragments containing small peptides of interest or prepared recombinantly for use as the immunizing antigen. The polypeptide or peptide used to immunize an animal can be derived from the transition of RNA or synthesized chemically, and can be conjugated to a carrier protein, if desired. Commonly used carriers that are chemically coupled to peptides include bovine serum albumin and thyroglobulin. The coupled peptide is then used to immunize the animal, e.g., a mouse, a rat, or a rabbit.

The term “humanized antibody”, as used herein, refers to antibody molecules in which amino acids have been replaced in the non-antigen binding regions in order to more closely resemble a human antibody, while still retaining the original binding ability.

The deposit(s) referred to herein will be maintained under the terms of the Budapest Treaty on the International Recognition of the Deposit of Microorganisms for purposes of Patent Procedure. These deposits are provided merely as convenience to those of skill in the art and are not an admission that a deposit is required under 35 U.S.C. §112. The sequence of the polynucleotides contained in the deposited materials, as well as the amino acid sequence of the polypeptides encoded thereby, are incorporated herein by reference and are controlling in the event of any conflict with any description of sequences herein. A license may be required to make, or sell the deposited materials, and no such license is hereby granted.

The invention is novel human imidazoline receptors referred to as IMRRP1 and IMRRP1b, polynucleotides encoding IMRRP1 and IMRRP1b, and the use of these compositions for the diagnosis, prevention, or treatment of disorders associated with aberrant cellular development, immune responses and inflammation, as well as organ and tissue transplantation rejection.

Human imidazoline receptor protein sequence was used as a probe to search the Incyte and public domain EST databases. The search program used was gapped BLAST (Altschul et al., 1997). The top EST hits from the BLAST results were searched back against the non-redundant protein and patent sequence databases. From this analysis, ESTs encoding a potential novel imidazoline receptor was identified based on sequence homology. The Incyte EST (Clone ID: 2499870) was selected as a potential novel imidazoline receptor candidate for subsequent analysis.

A PCR primer pair, designed from the DNA sequence of Incyte clone-2499870 was used to amplify a piece of DNA from the clone in which the anti-sense strand of the amplified fragment was biotinylated on the 5′ end. This biotinylated piece of double stranded DNA was denatured and incubated with a mixture of single-stranded covalently closed circular cDNA libraries which contain DNA corresponding to the sense strand. The cDNA libraries were total brain tissue libraries obtained from Gibco Life Technologies. Hybrids between the biotinylated DNA and the circular cDNA were captured on streptavidin magnetic beads. Upon thermal release of the cDNA from the biotinylated DNA, the single stranded cDNA was converted into double strands using a primer homologous to a sequence on the cDNA cloning vector. The double stranded cDNA was introduced into E. coli by electroporation and the resulting colonies were screen by PCR, using the original primer pair to identify the proper cDNA clones. One clone named FL1-18 was sequenced on both strands (FIGS. 1A-C). The deduced amino acid sequence corresponding to the nucleic acid sequence of clone FL1-18 is shown in FIGS. 1A-C.

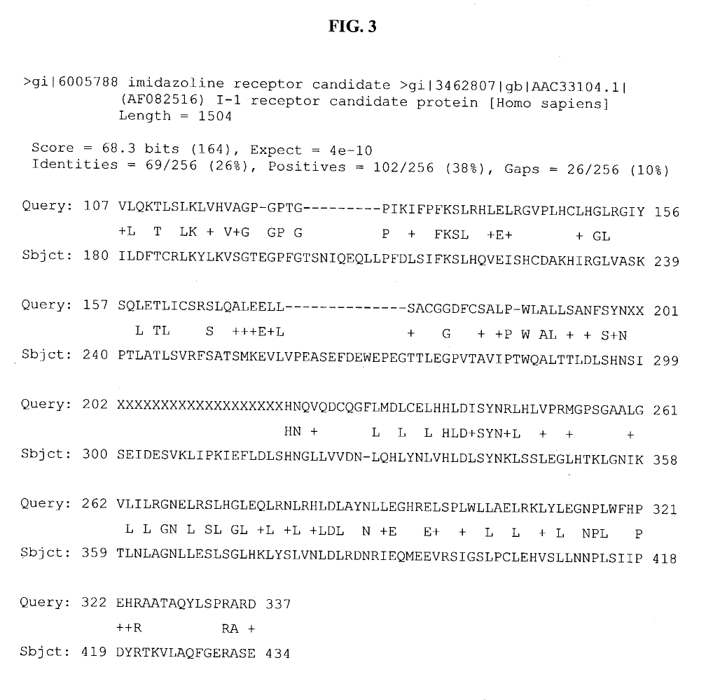

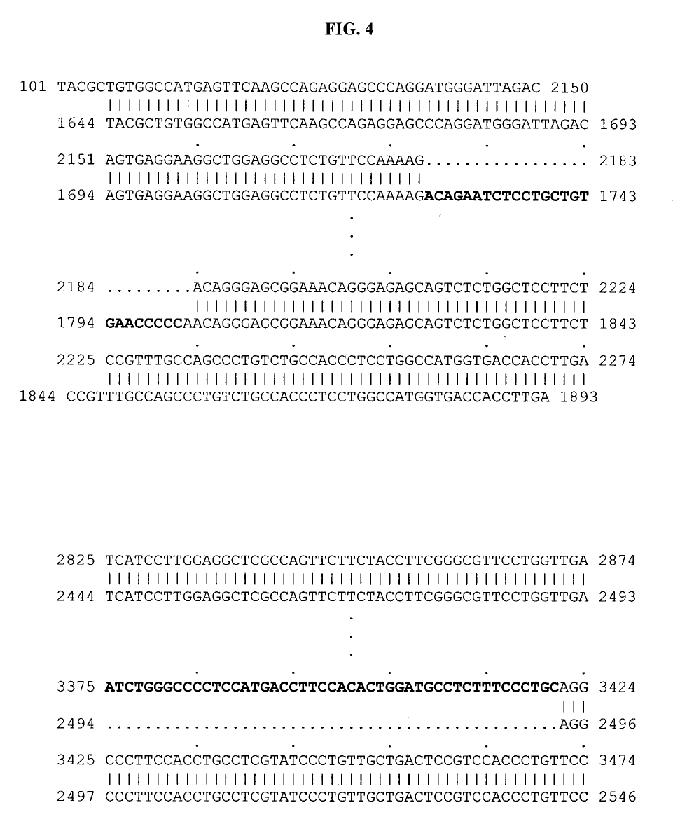

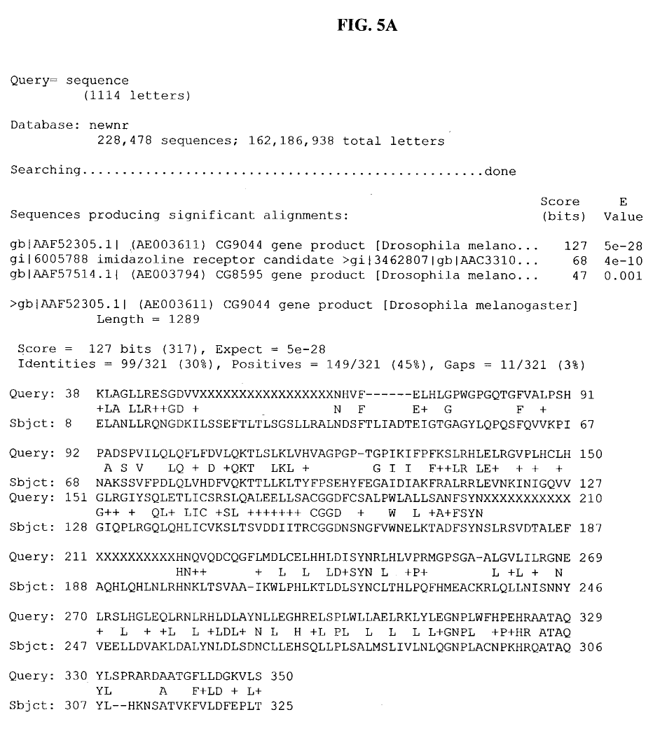

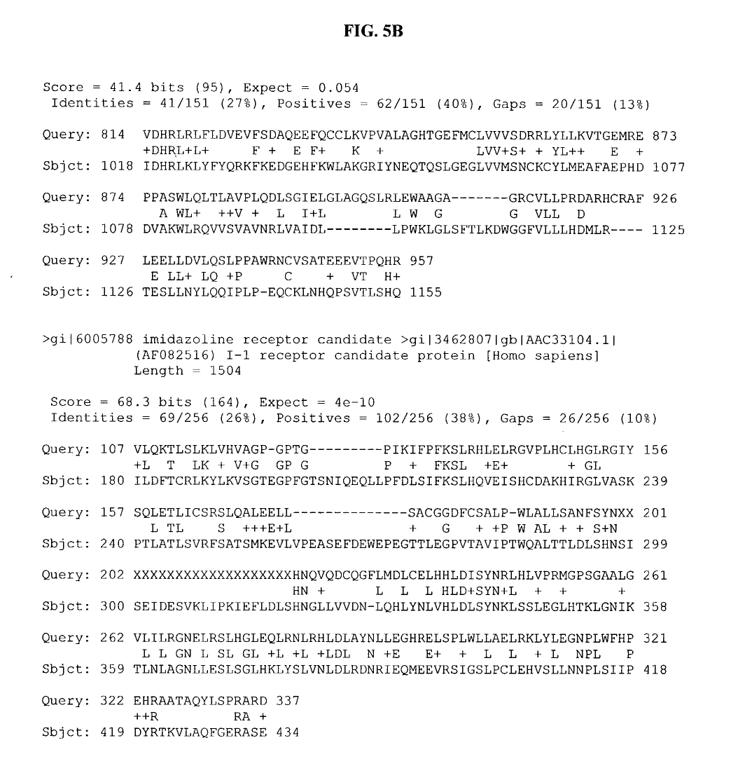

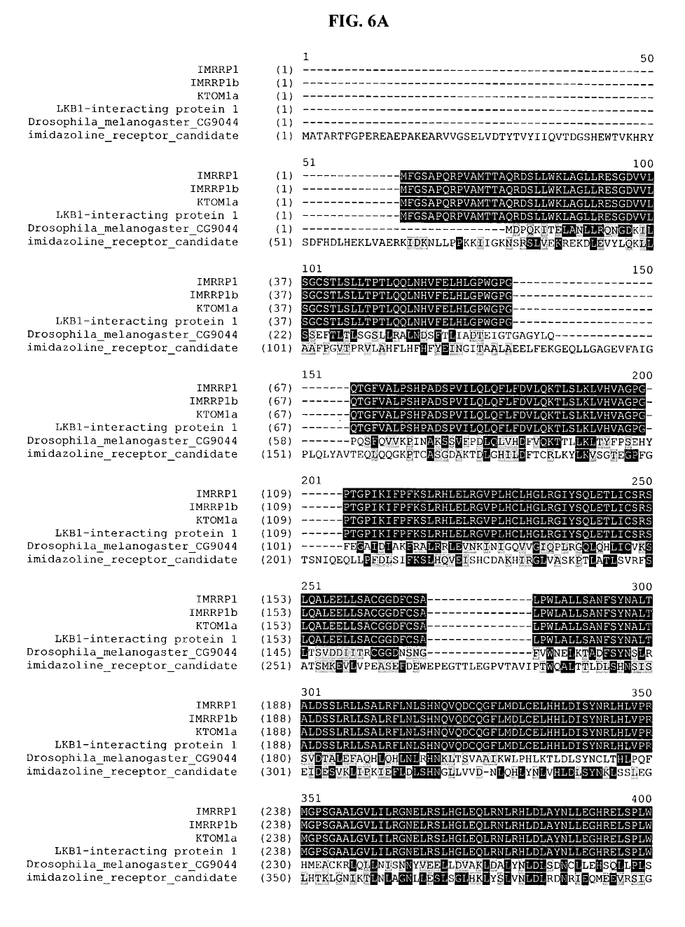

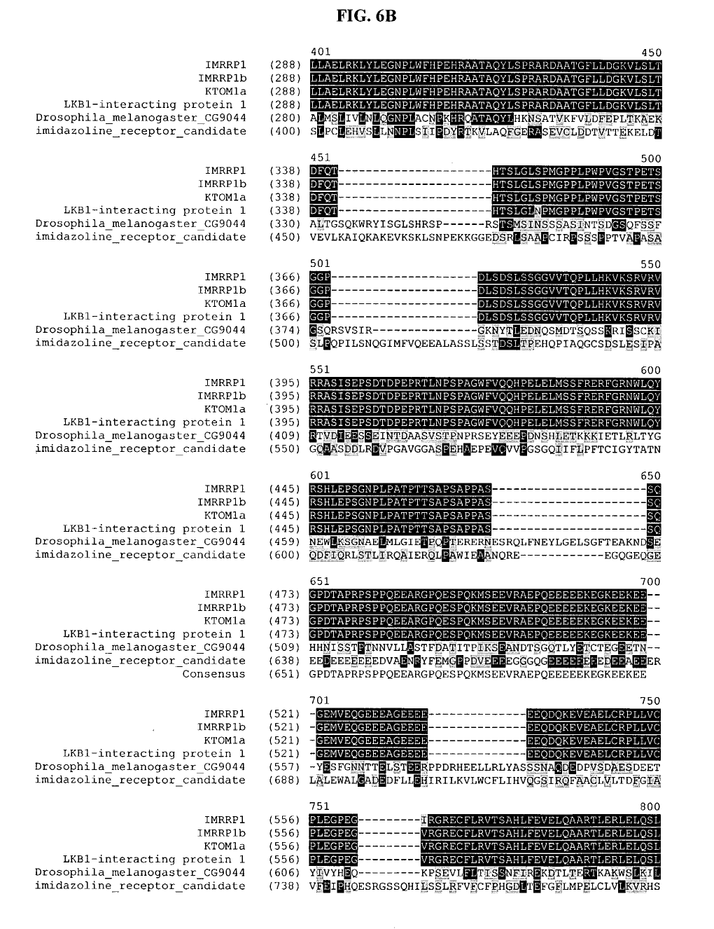

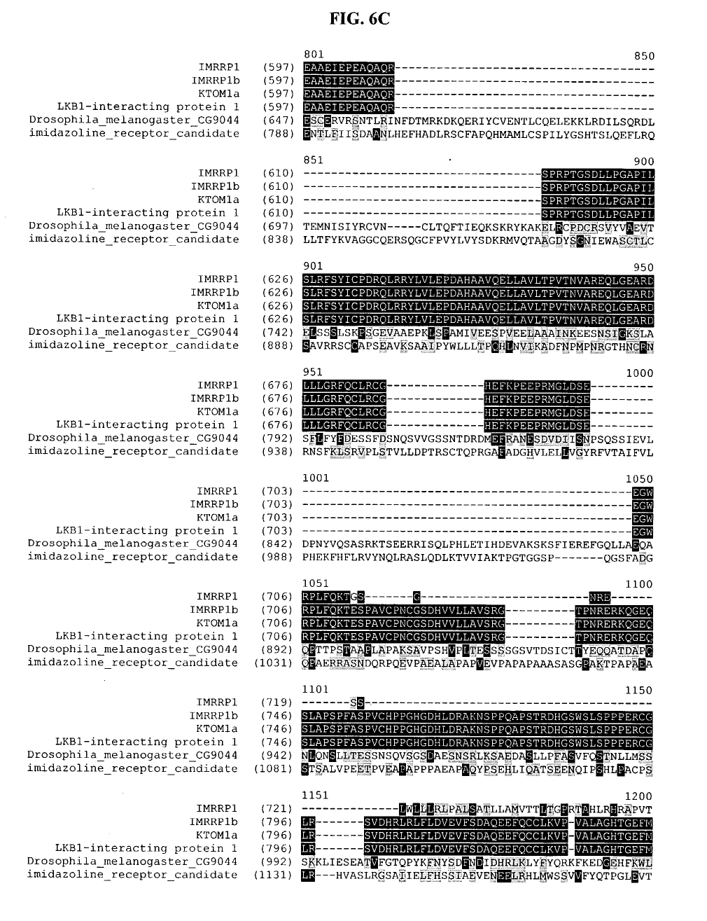

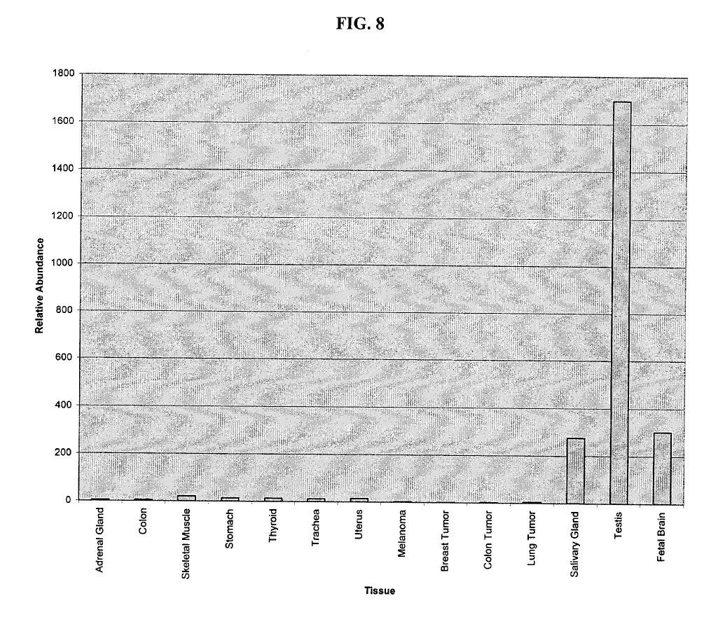

A comparison of the FL1-18 cDNA to that of the partial clone found in the Incyte database (clone 2499870) revealed that at nucleotide position 1725 of the Incyte clone a small insertion of 25 bases occurs and at position 3375 of clone FL1-18 an insertion of 47 bases occurs. (See FIG. 4: Top strand, FL1-18; bottom strand, Incyte 2499870.) An alignment of the two DNA sequences, FL1-18 and Incyte 2499870 is shown in FIG. 8.

An alignment of the two DNA sequences, FL1-18 and Incyte 2499870 to the rough draft of the human genome, revealed that both insertions/deletion in the two sequences correspond to putative exons as determined by the conservation of splice donor and acceptor sequences on either side of the inserted DNA and hence represent different RNA splice forms of a transcript that originates from one genomic location (i.e., one polynucleotide).

The Incyte clone is missing approximately 450 bp of the 5′-end. Combining the 5′-end sequences of FL1-18 sequence with that of the Incyte clone creates a novel nucleotide sequence which is referred to the FL1-18 splice variant. Translation of this sequence produces a longer polypeptide chain than that of FL1-18 because of the elimination of an in frame stop caused by the lack of the small exon in FL1-18. The first 712 amino acid are identical, but after that the remaining 97 amino acids of FL1-18 differ. The second alternatively spliced exon found in the Incyte clone is a coding exon. Hence, these splice variants produce different length and possibly different functional proteins.

In one embodiment, the invention encompasses a polypeptide comprising the amino acid sequence of SEQ ID NO:3 as shown in FIGS. 1A-C, or the amino acid sequence of SEQ ID NO:4 as shown in FIGS. 2A-D. IMRRP1 and IMRRP1b share chemical and structural homology with the human imidazoline receptor, Accession number NP_{—}009115. IMRRP1 and IMRRP1b also share chemical and structural homology with two Drosophila proteins identified as Accession number AAF52305 and Accession number AAF57514. IMRRP1 shares 26% identity with the human imidazoline receptor, Accession number NP_{—}009115, as illustrated in FIG. 3.

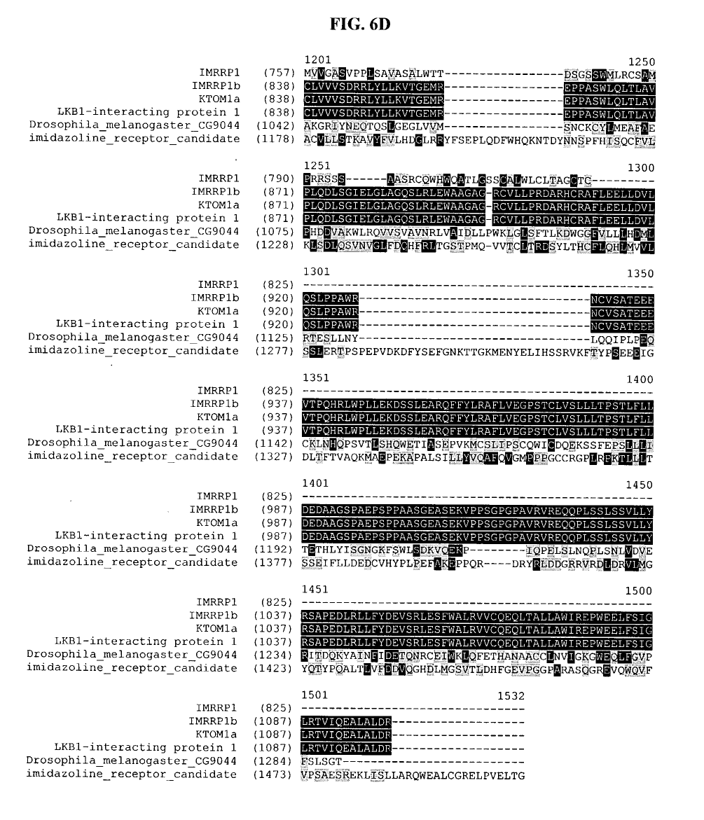

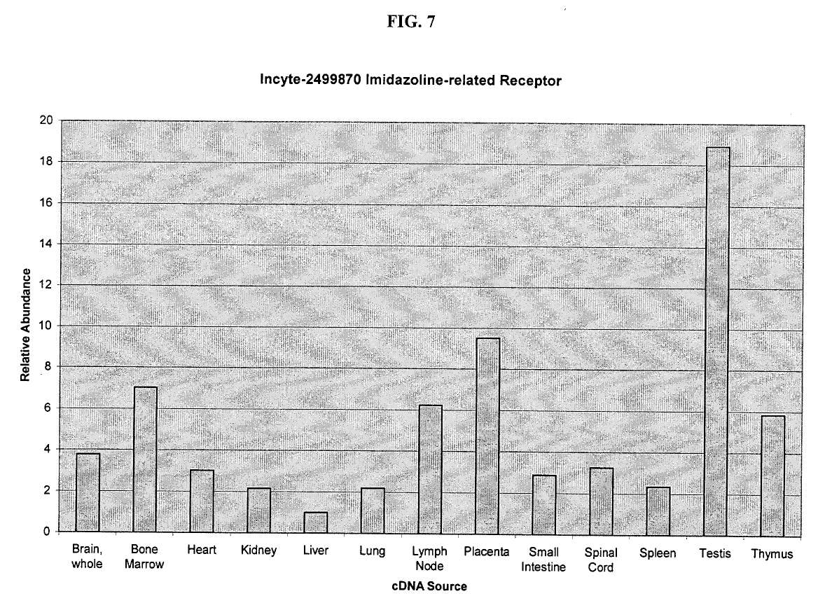

Expression profiling of imidazoline receptor homolog IMRRP1 showed expression in a variety of human tissue, most notably in testis tissue. The same PCR primer used in the cloning of imidazoline receptor IMRRP1 was used to measure the steady state levels of mRNA by quantitative PCR. Briefly, first strand cDNA was made from commercially available mRNA. The relative amount of cDNA used in each assay was determined by performing a parallel experiment using a primer pair for a polynucleotide expressed in equal amounts in all tissues, cyclophilin. The cyclophilin primer pair detected small variations in the amount of cDNA in each sample and these data were used for normalization of the data obtained with the primer pair for IMRRP1. The PCR data was converted into a relative assessment of the difference in transcript abundance amongst the tissues tested and the data is presented in FIGS. 7 and 8.

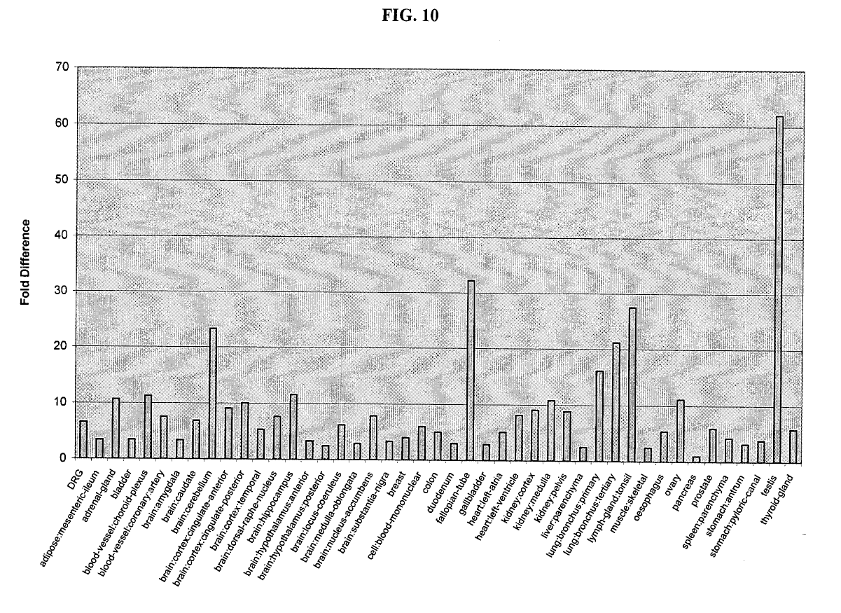

Expanded analysis of IMRRP1 expression levels by TaqMan™ quantitative PCR (see FIG. 10) confirmed that the IMRRP1 polypeptide is expressed in testis and lymph node as demonstrated initially in FIG. 7). IMRRP1 mRNA was expressed predominately in testis, significantly in fallopian tube, lymph gland, lung, brain, and to a lesser extent in other tissues.

IMRRP1 polynucleotides and polypeptides are useful for treating, diagnosing, prognosing, and/or preventing testicular, in addition to other male reproductive disorders. In preferred embodiments, IMRRP1 polynucleotides and polypeptides including agonists and fragments thereof, have uses which include treating, diagnosing, prognosing, and/or preventing the following, non-limiting, diseases or disorders of the testis: spermatopolynucleotidesis, infertility, Klinefelter's syndrome, XX male, epididymitis, genital warts, germinal cell aplasia, cryptorchidism, varicocele, immotile cilia syndrome, and viral orchitis. The IMRRP1 polynucleotides and polypeptides including agonists and fragements thereof, may also have uses related to modulating testicular development, embryopolynucleotidesis, reproduction, and in ameliorating, treating, and/or preventing testicular proliferative disorders (e.g., cancers, which include, for example, choriocarcinoma, Nonseminoma, seminona, and testicular germ cell tumors).

Likewise, the predominate localized expression in testis tissue also emphasizes the potential utility for IMRRP1 polynucleotides and polypeptides in treating, diagnosing, prognosing, and/or preventing metabolic diseases and disorders which include the following, not limiting examples: premature puberty, incomplete puberty, Kallman syndrome, Cushing's syndrome, hyperprolactinemia, hemochromatosis, congenital adrenal hyperplasia, FSH deficiency, and granulomatous disease, for example.

This IMRRP1 polypeptide may also be useful in assays designed to identify binding agents, as such agents (antagonists) are useful as male contraceptive agents. The testes are also a site of active polynucleotide expression of transcripts that is expressed, particularly at low levels, in other tissues of the body. Therefore, this polypeptide may be expressed in other specific tissues or organs where it may play related functional roles in other processes, such as hematopoiesis, inflammation, bone formation, and kidney function, to name a few possible target indications.

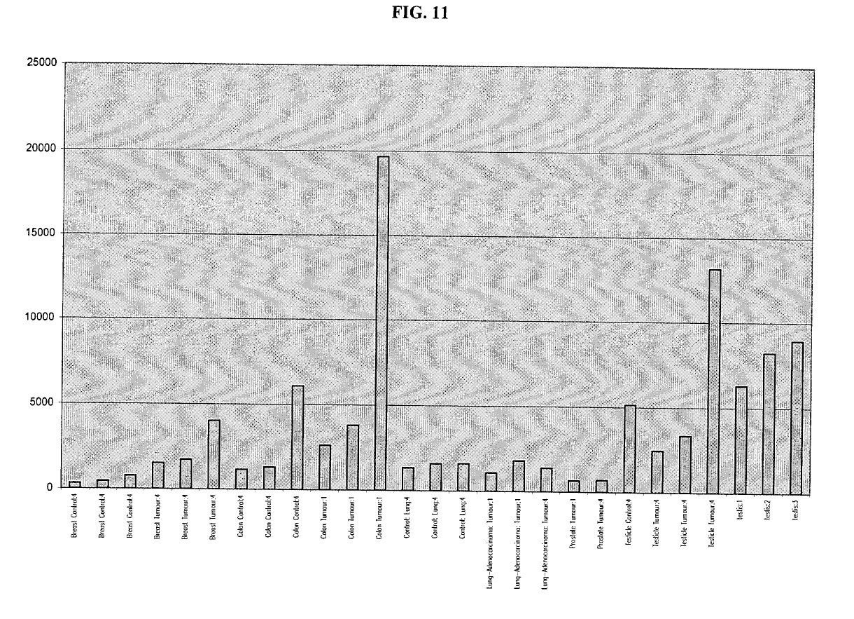

Morever, an additional analysis of IMRRP1 expression levels by TaqMan™ quantitative PCR (see FIG. 11) in disease cells and tissues indicated that the IMRRP1 polypeptide is differentially expressed in testicular tumor tissues, colon cancer tissues, and in breast tumor tissues. These data support a role of IMRRP1 in regulating various proliferative functions in the cell, particularly cell cycle regulation in a number of tissues and cell types. Small molecule modulators of IMRRP1 function may represent a novel therapeutic option in the treatment of proliferative diseases and disorders, particularly cancers and tumors of the testis, breast, and colon.

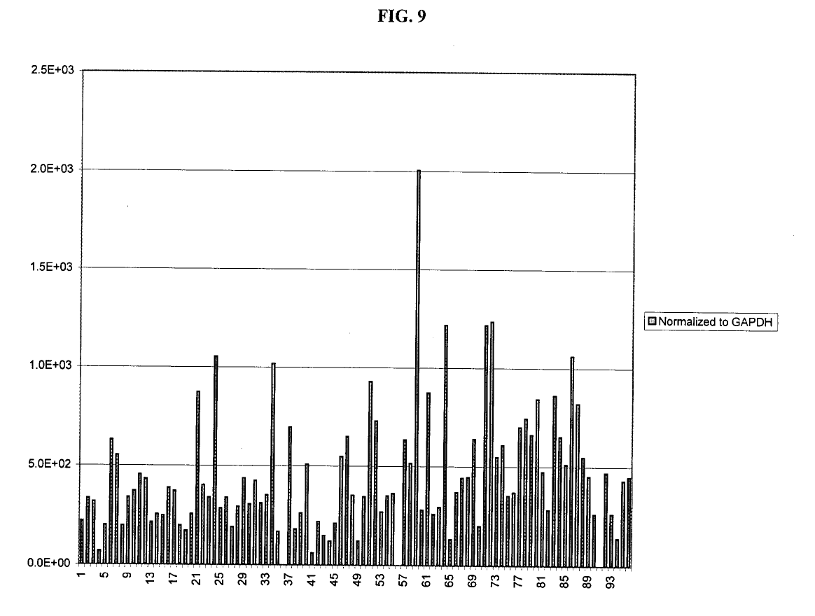

Additional expression profiling analysis of IMRRP1 expression levels in various cancer cell lines by TaqMan™ quantitative PCR (see FIG. 9) determined that IMRRP1 is expressed in several lung and breast cancer cell lines. The data suggests the IMRRP1 polypeptide may play a critical role in the development of a transformed phenotype leading to the development of cancers and/or a proliferative condition, either directly or indirectly. Alternatively, the IMRRP1 polypeptide may play a protective role and could be activated in response to a cancerous or proliferative phenotype. Whether IMRRP1 plays a role in directing transformation, or plays the role of protecting cells in response to a transformed phenotype, its role in ovarian tumors is likely to be enhanced relative to normal tissues. Therefore, antagonists or agonists of the IMRRP1 polypeptide may be useful in the treatment, amelioration, and/or prevention of a variety of proliferative conditions, including, but not limited to lung, and breast tumors.

A polypeptide sequence sharing 99% sequence identity to the IMRRP1b polypeptide, entitled LkB1-interacting protein 1 (Genbank Accession No. gi|17940700; SEQ ID NO:34) recently published. LkB1 is described as a serine/threonine kinase associated with Peutz-Jeghers syndrome (PJS), a condition characterized by multiple gastrointestinal hamartomatous polyps. Patients with PJS are 10 times more likely to develop cancer than the polynucleotideral population, particularly of the colon, small intestine, breast, cervix, ovary, and pancreas (Smith, D. P., et al., Hum. Mol. Genet. 10(25):2869-2877 (2001).

Based upon the identity between the IMRRP1b polypeptide to the LkB1-interacting protein 1, it is likely that the alternative splice form of IMRRP1b, the IMRRP1 polypeptide (SEQ ID NO:3), is also associated with the incidence of Peutz-Jeghers syndrome, in addition to cancers, particularly of the colon, small intestine, breast, cervix, ovary, and pancreas.

The association of IMRRP1 to proliferative disorders is consistent with the expression profiles described herein whereby IMRRP1 was differentially expressed in breast, testicular, and colon cancer cell lines.

Another polypeptide sequence sharing 100% sequence identity to the IMRRP 1b polypeptide, entitled KMOT1a protein (International Publication No. WO 02/24750; SEQ ID NO:33) also recently published. KMOT1a is described as a polypeptide associated with kidney tumors.

The independent association of the IMRRP1b polypeptide to the incidence of another cancer type, kidney tumors, further supports the association of the IMRRP1 polypeptide (SEQ ID NO:3) to the incidence of proliferative disorders.

As described more particularly herein, the IMRRP1 polypeptide was also shown to be associated with modulating the expression and/or activity of the p27 cell-cycle check point polypeptide, in addition to the inflammatory/apoptosis regulator, IkB.

Collectively, the data suggests that the IMRRP1 polypeptide is integrally involved in the incidence of a proliferative state in cells and tissues and that modulators of IMRRP1 may provide therapeutic benefit. Specifically, IMRRP1 polynucleotides (SEQ ID NO:1), polypeptides (SEQ ID NO:3), including fragments and modulators thereof, are useful for the treatment, amelioration, and/or detection of a number of proliferative disorders, particularly tumors, polyps, and cancers of the colon, small intestine, breast, cervix, ovary, pancreas, and kidney.

Characterization of the IMRRP1 and/or IMRRP1b polypeptides of the present invention using antisense oligonucleotides led to the determination that IMRRP1 and/or IMRRP1b are involved in the negative modulation of the p21 G1/G2 cell cycle check point modulatory protein as described in Example IX herein.

These results suggest that inhibition of IMRRP1 and/or IMRRP1b activity or expression with a modulator would induce differentiation, and stop cellular proliferation, as p21 is a cell cycle inhibitor and is known to be associated with committment down a differentiation pathway. Numerous known drugs in clinical trials (such as, for example, cdk2 inhibitors, dna methyltransferase inhibitors) also induce p21, and have been shown to have activity in patients with cancer. Thus, p21 induction is a plausable marker of anticancer potential when a target is appropriately modulated.

In preferred embodiments, IMRRP1 and/or IMRRP1b polynucleotides and polypeptides, including modulators and fragments thereof, are useful for treating, diagnosing, and/or ameliorating cell cycle defects, disorders related to aberrant phosphorylation, disorders related to aberrant signal transduction, proliferating disorders, and/or cancers.

Moreover, IMRRP1 and/or IMRRP1b polynucleotides and polypeptides, including modulators and fragments thereof, are useful for decreasing, or alternatively increasing, cellular proliferation; decreasing, or alternatively increasing, cellular proliferation in rapidly proliferating cells; increasing, or alternatively decreasing, the number of cells in the G1 phase of the cell cycle; increasing, or alternatively decreasing, the number of cells in the G2 phase of the cell cycle; decreasing, or alternatively increasing, the number of cells that progress to the S phase of the cell cycle; decreasing, or alternatively increasing, the number of cells that progress to the M phase of the cell cycle; modulating DNA repair, and increasing, or alternatively decreasing, hematopoietic stem cell expansion.

Moreover, antagonists, or alternatively agonists, directed against IMRRP1 and/or IMRRP1b are useful for decreasing, or alternatively increasing, cellular proliferation; decreasing, or alternatively increasing, cellular proliferation in rapidly proliferating cells; increasing, or alternatively decreasing, the number of cells in the G1 phase of the cell cycle; increasing, or alternatively decreasing, the number of cells in the G2 phase of the cell cycle; decreasing, or alternatively increasing, the number of cells that progress to the S phase of the cell cycle; decreasing, or alternatively increasing, the number of cells that progress to the M phase of the cell cycle; and inducing, or alternatively inhibiting, cells into G1 and/or G2 phase arrest. Such antagonists, or alternatively agonists, would be particularly useful for transforming transformed cells to normal cells.

Characterization of the IMRRP1 and IMRRP1b polypeptide of the present invention using antisense oligonucleotides led to the determination that IMRRP1 and IMRRP1b are involved in the positive modulation of the p21 G1/G2 cell cycle check point modulatory protein as described in Example IX herein.

These results suggest that induction of IMRRP1 and/or IMRRP1b activity or expression with a modulator would induce differentiation, and stop cellular proliferation, as p21 is a cell cycle inhibitor and is known to be associated with committment down a differentiation pathway. Numerous known drugs in clinical trials (such as, for example, cdk2 inhibitors, dna methyltransferase inhibitors) also induce p21, and have been shown to have activity in patients with cancer. Thus, p21 induction is a plausable marker of anticancer potential when a target is appropriately modulated.

In preferred embodiments, IMRRP1 and/or IMRRP1b polynucleotides and polypeptides, including fragments thereof, are useful for treating, diagnosing, and/or ameliorating cell cycle defects, disorders related to aberrant phosphorylation, disorders related to aberrant signal transduction, proliferating disorders, and/or cancers.

Moreover, IMRRP1 and/or IMRRP1b polynucleotides and polypeptides, including fragments thereof, are useful for decreasing cellular proliferation, decreasing cellular proliferation in rapidly proliferating cells, increasing the number of cells in the G1 phase of the cell cycle, increasing the number of cells in the G2 phase of the cell cycle, decreasing the number of cells that progress to the S phase of the cell cycle, decreasing the number of cells that progress to the M phase of the cell cycle, modulating DNA repair, and increasing hematopoietic stem cell expansion.

In preferred embodiments, agonists directed to IMRRP1 and/or IMRRP1b are useful for decreasing cellular proliferation, decreasing cellular proliferation in rapidly proliferating cells, increasing the number of cells in the G1 phase of the cell cycle, increasing the number of cells in the G2 phase of the cell cycle, decreasing the number of cells that progress to the S phase of the cell cycle, decreasing the number of cells that progress to the M phase of the cell cycle, modulating DNA repair, and increasing hematopoietic stem cell expansion.

IMRRP1 and/or IMRRP1b polynucleotides and polypeptides, including fragments and agonists thereof, are useful for treating, preventing, or ameliorating proliferative disorders in a patient in need of treatment, such as cancer patients, particularly patients that have proliferative immune disorders such as leukemia, lymphomas, multiple myeloma, etc.

Moreover, antagonists directed against IMRRP1 and/or IMRRP1b are useful for increasing cellular proliferation, increasing cellular proliferation in rapidly proliferating cells, decreasing the number of cells in the G1 phase of the cell cycle, decreasing the number of cells in the G2 phase of the cell cycle, increasing the number of cells that progress to the S phase of the cell cycle, increasing the number of cells that progress to the M phase of the cell cycle, and releasing cells from G1 and/or G2 phase arrest. Such antagonists would be particularly useful for transforming normal cells into immortalized cell lines, stimulating hematopoietic cells to grow and divide, increasing recovery rates of cancer patients that have undergone chemotherapy or other therapeutic regimen, by boosting their immune responses, etc. In addition, such antagonists of IMRRP1 and/or IMRRP1b would also be useful for repolynucleotiderating neural tissues (e.g., treatment of Parkinson's or Alzheimers patients with neural stem cells, or neural cells that have been activated by an IMRRP1 and/or IMRRP1b antagonist).

Characterization of the IMRRP1 and IMRRP1b polypeptide of the present invention using antisense oligonucleotides led to the determination that IMRRP1 or IMRRP1b is involved in modulation of the NFkB pathway through the negative modulation of the IkB modulatory protein as described in Example X herein.

In preferred embodiments, IMRRP1 or IMRRP1b polynucleotides and polypeptides, including modulators and fragments thereof, are useful for treating, diagnosing, and/or ameliorating proliferative disorders, cancers, ischemia-reperfusion injury, heart failure, immuno compromised conditions, HIV infection, and renal diseases.

Moreover, IMRRP1 or IMRRP1b polynucleotides and polypeptides, including modulators and fragments thereof, are useful for decreasing NF-kB activity, increasing apoptotic events, and/or increasing IκBα expression or activity levels.

In preferred embodiments, antagonists directed against IMRRP1 and/or IMRRP1b are useful for treating, diagnosing, and/or ameliorating autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, and HIV propagation in cells infected with other viruses.

Moreover, antagonists directed against IMRRP1 and/or IMRRP1b are useful for decreasing NF-kB activity, increasing apoptotic events, and/or increasing IκBα expression or activity levels.

In preferred embodiments, agonists directed against IMRRP I and/or IMRRP1b are useful for treating, diagnosing, and/or ameliorating autoimmune diorders, disorders related to hyper immune activity, hypercongenital conditions, birth defects, necrotic lesions, wounds, disorders related to aberrant signal transduction, immuno compromised conditions, HIV infection, proliferating disorders, and/or cancers.

Moreover, agonists directed against IMRRP1 and/or IMRRP1b are useful for increasing NF-kB activity, decreasing apoptotic events, and/or decreasing IκBα expression or activity levels.

Characterization of the IMRRP1 and/or IMRRP1b polypeptide of the present invention using antisense oligonucleotides led to the determination that IMRRP1 and/or IMRRP1b is involved in modulation of the NFkB pathway through the positive modulation of the IkB modulatory protein as described in Example X herein.

In preferred embodiments, IMRRP1 and/or IMRRP1b polynucleotides and polypeptides, including modulators and fragments thereof, are useful for treating, diagnosing, and/or ameliorating proliferative disorders, cancers, ischemia-reperfusion injury, heart failure, immuno compromised conditions, HIV infection, and renal diseases.

Moreover, IMRRP1 and/or IMRRP1b polynucleotides and polypeptides, including modulators and fragments thereof, are useful for increasing NF-kB activity, decreasing apoptotic events, and/or decreasing IκBα expression or activity levels.

In preferred embodiments, agonists directed against IMRRP 1 and/or IMRRP1b are useful for treating, diagnosing, and/or ameliorating autoimmune disorders, disorders related to hyper immune activity, inflammatory conditions, disorders related to aberrant acute phase responses, hypercongenital conditions, birth defects, necrotic lesions, wounds, organ transplant rejection, conditions related to organ transplant rejection, disorders related to aberrant signal transduction, proliferating disorders, cancers, HIV, and HIV propagation in cells infected with other viruses.

Moreover, agonists directed against IMRRP1 and/or IMRRP1b are useful for decreasing NF-kB activity, increasing apoptotic events, and/or increasing IκBα expression or activity levels.

In preferred embodiments, antagonists directed against IMRRP1 and/or IMRRP1b are useful for treating, diagnosing, and/or ameliorating autoimmune diorders, disorders related to hyper immune activity, hypercongenital conditions, birth defects, necrotic lesions, wounds, disorders related to aberrant signal transduction, immuno compromised conditions, HIV infection, proliferating disorders, and/or cancers.

Moreover, antagonists directed against IMRRP1 and/or IMRRP1b are useful for increasing NF-kB activity, decreasing apoptotic events, and/or decreasing IκBα expression or activity levels.

In preferred embodiments, immidazoline receptor polynucleotides and polypeptides, including fragments thereof, are useful for treating, diagnosing, and/or ameliorating cell cycle defects, disorders related to aberrant phosphorylation, disorders related to aberrant signal transduction, proliferating disorders, and/or cancers.

Moreover, immidazoline receptor polynucleotides and polypeptides, including fragments thereof, are useful for decreasing cellular proliferation, decreasing cellular proliferation in rapidly proliferating cells, increasing the number of cells in the G1 phase of the cell cycle, and decreasing the number of cells that progress to the S phase of the cell cycle.

In preferred embodiments, agonists directed to immidazoline receptor are useful for decreasing cellular proliferation, decreasing cellular proliferation in rapidly proliferating cells, increasing the number of cells in the G1 phase of the cell cycle, and decreasing the number of cells that progress to the S phase of the cell cycle.

Moreover, antagonists directed against immidazoline receptor are useful for increasing cellular proliferation, increasing cellular proliferation in rapidly proliferating cells, decreasing the number of cells in the G1 phase of the cell cycle, and increasing the number of cells that progress to the S phase of the cell cycle. Such antagonists would be particularly useful for transforming normal cells into immortalized cell lines, stimulating hematopoietic cells to grow and divide, increasing recovery rates of cancer patients that have undergone chemotherapy or other therapeutic regimen, by boosting their immune responses, etc.

As described herein, antisense reagents to IMRRP1 results in induction of P21 and IkB. These results suggest that IMRRP1 is involved in a pathway that controls a cells commitment to differentiation that is also involved in driving the cell into apoptosis as well. Controlling such a pathway would be favorable in cancer therapy, as it should result in cell death and impact the disease in a positive way if the IMRRP1 polypeptide were to be inhibited in a patient. An antagonist of IMRRP1 would be preferred for cancer therapy.

The invention also encompasses IMRRP1 and IMRRP1b variants. Preferred IMRRP1 and IMRRP1b variants are those having at least 80%, and more preferably 90% or greater, amino acid identity to the IMRRP1 and IMRRP1b amino acid sequence of SEQ ID NOS: 3 and 4, respectively Most preferred IMRRP1 and IMRRP1b variants are those having at least 95% amino acid sequence identity to SEQ ID NOS: 3 and 4, respectively.

The present invention provides isolated IMRRP1 and IMRRP1b and homologs thereof. Such proteins are substantially free of contaminating endogenous materials and, optionally, without associated nature-pattern glycosylation. Derivatives of the IMRRP1 and IMRRP1b receptors within the scope of the invention also include various structural forms of the primary protein which retain biological activity. Due to the presence of ionizable amino and carboxyl groups, for example, IMRRP1 and IMRRP1b proteins may be in the form of acidic or basic salts, or may be in neutral form. Individual amino acid residues may also be modified by oxidation or reduction.

The primary amino acid structure may be modified by forming covalent or aggregative conjugates with other chemical moieties, such as glycosyl groups, lipids, phosphate, acetyl groups and the like, or by creating amino acid sequence mutants. Covalent derivatives are prepared by linking particular functional groups to amino acid side chains or at the N- or C-termini.

The present invention further encompassed fusion proteins comprising the amino acid sequence of IMRRP1 or IMRRP1b or portions thereof linked to an immunoglobulin Fc region. Depending on the portion of the Fc region used, a fusion protein may be expressed as a dimer, through formation of interchain disulfide bonds. If the fusion proteins are made with both heavy and light chains of an antibody, it is possible to form a protein oligomer with as many as four IMRRP1 and/or IMRRP1b regions.

The invention also encompasses polynucleotides which encode IMRRP1 and IMRRP1b. Accordingly, any nucleic acid sequence which encodes the amino acid sequence of IMRRP1 or IMRRP1b can be used to polynucleotiderate recombinant molecules which express IMRRP1 and IMRRP1b. In a particular embodiment, the invention encompasses the polynucleotide comprising the nucleic acid sequence of SEQ ID NOS. 1 and 2 as shown in FIGS. 1 and 2.

It will be appreciated by those skilled in the art that as a result of the depolynucleotideracy of the polynucleotidetic code, a multitude of nucleotide sequences encoding IMRRP1 and IMRRP1b, some bearing minimal homology to the nucleotide sequences of any known and naturally occurring polynucleotide, may be produced. Thus, the invention contemplates each and every possible variation of nucleotide sequence that could be made by selecting combinations based on possible codon choices. These combinations are made in accordance with the standard triplet polynucleotidetic code as applied to the nucleotide sequence of naturally occurring IMRRP1 and IMRRP1b, and all such variations are to be considered as being specifically disclosed.

Although nucleotide sequences which encode IMRRP1 or IMRRP1b and their variants are preferably capable of hybridizing to the nucleotide sequence of the naturally occurring coding sequence for IMRRP1 or IMRRP1b under appropriately selected conditions of stringency, it may be advantageous to produce nucleotide sequences encoding IMRRP1 or IMRRP1b or their derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding IMRRP1 or IMRRP1b and their derivatives without altering the encoded amino acid sequences include the production of RNA transcripts having more desirable properties, such as a greater half-life, than transcripts produced from the naturally occurring sequence.

The invention also encompasses production of DNA sequences, or portions thereof, which encode IMRRP1 or IMRRP1b and their derivatives, entirely by synthetic chemistry. After production, the synthetic sequence may be inserted into any of the many available expression vectors and cell systems using reagents that are well known in the art at the time of the filing of this application. Moreover, synthetic chemistry may be used to introduce mutations into a sequence encoding IMRRP1 or IMRRP1b or any portion thereof.

Also encompassed by the invention are polynucleotide sequences that are capable of hybridizing to the claimed nucleotide sequences, and in particular, those shown in SEQ ID NOS: 1 and 2, under various conditions of stringency. Hybridization conditions are based on the melting temperature (Tm) of the nucleic acid binding complex or probe, as taught in Wahl, G. M. and S. L. Berger (1987; Methods Enzymol. 152:399-407) and Kimmel, A. R. (1987; Methods of Enzymol. 152:507-511), and may be used at a defined stringency. In one embodiment, sequences include those capable of hybridizing under moderately stringent conditions (prewashing solution of 2×SSC, 0.5% SOS, 1.0 mM MEDTA, pH 8.0) and hybridization conditions of 50° C., 5×SSC, overnight, to the sequences encoding IMRRP1 or IMRRP1b and other sequences which are depolynucleotiderate to those which encode IMRRP1 or IMRRP1b.

Altered nucleic acid sequences encoding IMRRP1 or IMRRP1b which are encompassed by the invention include deletions, insertions, or substitutions of different nucleotides resulting in a polynucleotide that encodes the same or a functionally equivalent IMRRP1 or IMRRP1b. The encoded protein may also contain deletions, insertions, or substitutions of amino acid residues which produce a silent change and result in a functionally equivalent IMRRP1 or IMRRP1b. Deliberate amino acid substitutions may be made on the basis of similarity in polarity, charge, solubility, hydrophobicity, hydrophilicity, and/or the amphipathic nature of the residues as long as the biological activity of IMRRP1 and IMRRP1b is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the polynucleotides encoding IMRRP1 and IMRRP1b. As used herein, an “allele” or “allelic sequence” is an alternative form of the polynucleotide which may result from at least one mutation in the nucleic acid sequence. Alleles may result in altered mRNAs or polypeptides whose structure or function may or may not be altered. Any given polynucleotide may have none, one, or many allelic forms. Common mutational changes which give rise to alleles are polynucleotiderally ascribed to natural deletions, additions, or substitutions of nucleotides. Each of these types of changes may occur alone, or in combination with the others, one or more times in a given sequence.

Methods for DNA sequencing which are well known and polynucleotiderally available in the art may be used to practice any embodiments of the invention. The methods may employ such enzymes as the Klenow fragment of DNA polymerase I, SEQUENCE (US Biochemical Corp. Cleveland, Ohio), Taq polymerase (Perkin Elmer), thermostable T7 polymerase (Amersham, Chicago, Ill.), or combinations of recombinant polymerases and proofreading exonucleases such as the ELONGASE Amplification System marketed by Gibco BRL (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI 377 DNA sequencers (Perkin Elmer).

The nucleic acid sequences encoding IMRRP1 or IMRRP9 may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). In particular, genomic DNA is first amplified in the presence of primer to linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia, T. et al. (1988) Nucleic Acids Res. 16:8186). The primers may be designed using OLIGO 4.06 Primer Analysis software (National Biosciences Inc., Plymouth, Minn.), or another appropriate program, to be 22-30 nucleotides in length, to have a GC content of 50% or more, and to anneal to the target sequence at temperatures about 68° C. to about 72° C. The method uses several restriction enzymes to polynucleotiderate a suitable fragment in the known region of a polynucleotide. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom, M. et al. (1991) PCR Methods Applic. 1:111-119). In this method, multiple restriction enzyme digestions and ligations may also be used to place an engineered double-stranded sequence into an unknown portion of the DNA molecule before performing PCR.

Another method which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences which contain the 5′ regions of polynucleotides. Use of a randomly primed library may be especially preferable for situations in which an oligo d(T) library does not yield a full-length cDNA. Genomic libraries may be useful for extension of sequence into the 5′ and 3′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled device camera. Output/light intensity may be converted to electrical signal using appropriate software (e.g., GENOTYPER and SEQUENCE NAVIGAT and/or, Perkin Elmer) and the entire process from loading of samples to computer analysis and electronic data display may be computer controlled. Capillary electrophoresis is especially preferable for the sequencing of small pieces of DNA which might be present in limited amounts in a particular sample.

In another embodiment of the invention, polynucleotide sequences or fragments thereof which encode or fusion proteins or functional equivalents thereof, may be used in recombinant DNA molecules to direct expression of IMRRP1 or IMRRP1b in appropriate host cells. Due to the inherent depolynucleotideracy of the polynucleotidetic code, other DNA sequences which encode substantially the same or a functionally equivalent amino acid sequence may be produced and these sequences may be used to clone and express IMRRP1 or IMRRP1b.

As will be understood by those of skill in the art, it may be advantageous to produce IMRRP1- or IMRRP1b-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript polynucleotiderated from the naturally occurring sequence.

The nucleotide sequences of the present invention can be engineered using methods polynucleotiderally known in the art in order to alter the IMRRP1 and IMRRP1b encoding sequences for a variety of reasons, including but not limited to, alterations which modify the cloning, processing, and/or expression of the polypeptide. DNA shuffling by random fragmentation and PCR reassembly of polynucleotide fragments and synthetic oligonucleotides may be used to engineer the nucleotide sequences. For example, site-directed mutapolynucleotidesis may be used to insert new restriction sites, alter glycosylation patterns, change codon preference, produce splice variants, or introduce mutations, and so forth.

In another embodiment of the invention, natural, modified, or recombinant nucleic acid sequences encoding IMRRP1 or IMRRP1b may be ligated to a heterologous sequence to encode a fusion protein. For example, to screen peptide libraries for inhibitors of IMRRP1 or IMRRP1b activity, it may be useful to encode a chimeric IMRRP1 or IMRRP1b protein that can be recognized by a commercially available antibody. A fusion protein may also be engineered to contain a cleavage site located between the IMRRP1 or IMRRP1b encoding sequence and the heterologous protein sequence, so that IMRRP1 or IMRRP1b may be cleaved and purified away from the heterologous moiety.

In another embodiment, sequences encoding IMRRP1 or IMRRP1b may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of IMRRP1 or IMRRP1b, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer).

The newly synthesized peptide may be substantially purified by preparative high performance liquid chromatography (e.g., Creighton, T. (1983) Proteins, Structures and Molecular Principles, W H Freeman and Co., New York, N.Y.), by reverse-phase high performance liquid chromatography, or other purification methods as are known in the art. The composition of the synthetic peptides may be confirmed by amino acid analysis or sequencing (e.g., the Edman degradation procedure; Creighton, supra). Additionally, the amino acid sequence of IMRRP1 or IMRRP1b, or any part thereof, may be altered during direct synthesis and/or combined using chemical methods with sequences from other proteins, or any part thereof, to produce a variant polypeptide.

In order to express a biologically active IMRRP1 or IMRRP1b the nucleotide sequences encoding IMRRP1 or IMRRP1b or functional equivalents, may be inserted into an appropriate expression vector, i.e., a vector which contains the necessary elements for the transcription and translation of the inserted coding sequence.

Methods which are well known to those skilled in the art may be used to construct expression vectors containing sequences encoding and appropriate transcriptional and translational control elements. These methods include in vitro recombinant DNA techniques, synthetic techniques, and in vivo polynucleotidetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilized to contain and express sequences encoding IMRRP1 or IMRRP1b. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV or tobacco mosaic virus, TMV) or with bacterial expression vectors (e.g., Ti or pBR322 plasmids); or animal cell systems.

The “control elements” or “regulatory sequences” are those non-translated regions of the vector-enhancers, promoters, 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratapolynucleotide, LaJolla, Calif.) or PSP and/or T1 plasmid (Gibco BILL) and the like may be used. The baculovirus polyhedrin promoter may be used in insect cells. Promoters or enhancers derived from the genomes of plant cells (e.g., heat shock, RUBISCO; and storage protein polynucleotides) or from plant viruses (e.g., viral promoters or leader sequences) may be cloned into the vector. In mammalian cell systems, promoters from mammalian polynucleotides or from mammalian viruses are preferable. If it is necessary to polynucleotiderate a cell line that contains multiple copies of the sequence encoding IMRRP1 or IMRRP1b, vectors based on SV40 or EBV may be used with an appropriate selectable marker.

In bacterial systems, a number of expression vectors may be selected depending upon the use intended for IMRRP1 or IMRRP1b. For example, when large quantities are needed for the induction of antibodies, vectors which direct high level expression of fusion proteins that are readily purified may be used. Such vectors include, but are not limited to, the multifunctional E. coli cloning and expression vectors such as BLUESCRIPT (Stratapolynucleotide), in which the sequence encoding IMRRP1 or IMRRP1b may be ligated into the vector in frame with sequences for the amino-terminal Met and the subsequent 7 residues of β-galactosidase so that a hybrid protein is produced, pIN vectors (Van Heeke, G. and S. M. Schuster (1989) J. Biol. Chem. 264:5503-5509); and the like. pGEX vectors (Promega, Madison, Wis.) may also be used to express foreign polypeptides, as fusion proteins with glutathione S-transferase (GST). In polynucleotideral, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding IMRRP1 or IMRRP1b may be driven by any of a number of promoters. For example, viral promoters such as the 35S and 19S promoters of CaMV may be used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of polynucleotiderally available reviews (see, for example, Hobbs, S. or Murry, L. E. in McGraw Hill Yearbook of Science and Technology (1992) McGraw Hill, New York, N.Y.; pp. 191-196).

An insect system may also be used to express IMRRP1 or IMRRP1b. For example, in one such system, Autographa californica nuclear polyhedrosis virus (AcNPV) is used as a vector to express foreign polynucleotides in Spodoptera frugiperda cells or in Trichoplusia larvae. The sequences encoding IMRRP1 or IMRRP1b may be cloned into a non-essential region of the virus such as the polyhedrin polynucleotide, and placed under control of the polyhedrin promoter. Successful insertion of IMRRP1 or IMRRP1b will render the polyhedrin polynucleotide inactive and produce recombinant virus lacking coat protein. The recombinant viruses may then be used to infect, for example, S. frugiperda cells or Trichoplusia larvae in which IMRRP1 or IMRRP1b may be expressed (Engelhard, E. K. et al. (1994) Proc. Nat. Acad. Sci. 91:3224-3227).

In mammalian host cells, a number of viral-based expression systems may be utilized. In cases where an adenovirus is used as an expression vector, sequences encoding IMRRP1 or IMRRP1b may be ligated into an adenovirus transcription/translation complex consisting of the late promoter and tripartite leader sequence. Insertion in a non-essential E1 or E3 region of the viral genome may be used to obtain a viable virus which is capable of expressing IMRRP1 or IMRRP1b in infected host cells (Logan, J. and Shenk, T. (1984) Proc. Natl. Acad. Sci. 81:3655-3659). In addition, transcription enhancers, such as the Rous, sarcoma virus (RSV) enhancer, may be used to increase expression in mammalian host cells.

Specific initiation signals may also be used to achieve more efficient translation of sequences encoding IMRRP1 or IMRRP1b. Such signals include the ATG initiation codon and adjacent sequences. In cases where sequences encoding IMRRP1 or IMRRP1b, their initiation codon, and upstream sequences are inserted into the appropriate expression vector, no additional transcriptional or translational control signals may be needed. However, in cases where only a coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is preferred. For example, cell lines which stably express IMRRP1 or IMRRP1b may be transformed using expression vectors which may contain viral origins of replication and/or endogenous expression elements and a selectable marker polynucleotide on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1980) Cell 22:817-23) polynucleotides which can be employed in t⁻ or aprt⁻ cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable polynucleotides have been described, for example, trpB, which allows cells to utilize indole in place of tryptophan, or hisd, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, β glucuronidase and its substrate GUS, and liciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker polynucleotide expression suggests that the polynucleotide of interest is also present, its presence and expression may need to be confirmed. For example, if the sequence encoding IMRRP1 or IMRRP1b is inserted within a marker polynucleotide sequence, recombinant cells containing sequences encoding can be identified by the absence of marker polynucleotide function. Alternatively, a marker polynucleotide can be placed in tandem with a sequence encoding IMRRP1 or IMRRP1b under the control of a single promoter. Expression of the marker polynucleotide in response to induction or selection usually indicates expression of the tandem polynucleotide as well.

Alternatively, host cells which contain the nucleic acid sequence encoding IMRRP1 or IMRRP1b and express IMRRP1 or IMRRP1b may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein.

The presence of polynucleotide sequences encoding IMRRP1 or IMRRP1b can be detected by DNA-DNA or DNA-RNA hybridization or amplification using probes or portions or fragments of polynucleotides encoding IMRRP1 or IMRRP1b. Nucleic acid amplification based assays involve the use of oligonucleotides or oligomers based on the sequences encoding IMRRP1 or IMRRP 1b to detect transformants containing DNA or RNA encoding IMRRP1 or IMRRP1b. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of IMRRP1 or IMRRP1b, using either polyclonal or monoclonal antibodies specific for the proteins are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on IMRRP1 or IMRRP1b is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding IMRRP1 or IMRRP1b include oligolabeling, nick translation, end-labeling or PCR amplification using a labeled nucleotide. Alternatively, the sequences encoding IMRRP1 or IMRRP1b, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio)). Suitable reporter molecules or labels, which may be used, include radionuclides, enzymes, fluorescent, chemiluminescent, or chromogenic agents as well as substrates, cofactors, inhibitors, magnetic particles, and the like.

Host cells transformed with nucleotide sequences encoding IMRRP1 or IMRRP1b may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode IMRRP1 or IMRRP1b may be designed to contain signal sequences which direct secretion of IMRRP1 or IMRRP1b through a prokaryotic or eukaryotic cell membrane. Other recombinant constructions may be used to join sequences encoding IMRRP1 or IMRRP1b to nucleotide sequence encoding a polypeptide domain which will facilitate purification of soluble proteins. Such purification facilitating domains include, but are not limited to, metal chelating peptides such as histidine tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and IMRRP1 or IMRRP1b may be used to facilitate purification. One such expression vector provides for expression of a fusion protein containing IMRRP1 or IMRRP1b and a nucleic acid encoding 6 histidine residues preceding a thioredoxin or an enterokinase cleavage site. The histidine residues facilitate purification on IMIAC (immobilized metal ion affinity chromatography) as described in Porath, J et al. (1992, Prot. Exp. Purif: 3:263-281) while the enterokinase cleavage site provides a means for purifying from the fusion protein. A discussion of vectors which contain fusion proteins is provided in Kroll, D. J. et al. 993; DNA Cell Biol. 12:441-453).

In addition to recombinant production, fragments of IMRRP1 or IMRRP1b may be produced by direct peptide synthesis using solid-phase techniques (Merrifiel J. (1963) J. Am. Chem. Soc. 85:2149-2154). Protein synthesis may be performed using manual techniques or by automation. Automated synthesis may be achieved, for example, using Applied Biosystems 431 A Peptide Synthesizer (Perkin Elmer). Various fragments of IMRRP1 or IMRRP1b can be chemically synthesized separately and combined using chemical methods to produce the full length molecule Chemical and structural homology exists among IMRRP1 or IMRRP1b and the human imidazoline receptor disclosed in DNA Cell Biol. 19 (6), 319-329 (2000). Furthermore, IMRRP1 and IMRRP1b are expressed in brain, bone marrow, heart, kidney, liver, lung, lymph node, placenta, small intestine, spinal cord, spleen testis, and thymus tissues, many of which are associated with the regulation of blood pressure, induction of feeding, stimulation of firing of locus coeruleus neurons, and stimulation of insulin release, as well as the induction of the expression of glial fibrillary acidic protein independent of the action of alpha-2 adrenoceptors, dysphoric premenstrual syndrome, neurodepolynucleotiderative disorders such as Alzheimer's disease, opiate addiction, monoamine turnover and therefore nociception, ageing, mood and stroke, salivary disorders and developmental disorders. IMRRP1 and IMRRP1b therefore play an important role in mammalian physiology.

In another embodiment a vector capable of expressing IMRRP1 or IMRRP1b, or a fragment or derivative thereof, may also be administered to a subject to treat or prevent a physical or psychological disorder, including those listed above.

In another embodiment, agonists or antagonists of IMRRP1 or IMRRP1b may be administered to a subject to treat or prevent a disorder associated with many neurological conditions and disorders including depression. In one aspect, antibodies which are specific for IMRRP1 or IMRRP1b may be used directly as an antagonist, or indirectly as a targeting or delivery mechanism for bringing a pharmaceutical agent to cells or tissue which express IMRRP1 or IMRRP1b.

In another embodiment, a vector expressing the complementary or antisense sequence of the polynucleotide encoding IMRRP1 or IMRRP1b may be administered to a subject to treat or prevent a disorder associated many neurological conditions and disorders including depression.

In another embodiment a vector expressing the complementary or antisense sequence of the polynucleotide encoding IMRRP1 or IMRRP1b may be administered to a subject to treat or many neurological conditions and disorders including depression associated with expression of IMRRP1 or IMRRP1b.

In other embodiments, any of the therapeutic proteins, antagonists, antibodies, agonists, antisense sequences or vectors described above may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Agonists and antagonists or inhibitors of IMRRP1 or IMRRP1b may be produced using methods which are polynucleotiderally known in the art. For example, cloned receptors may be expressed in mammalian cells and compounds can be screened for activity. In addition, purified IMRRP1 or IMRRP1b may be used to produce antibodies or to screen libraries of pharmaceutical agents to identify those which specifically bind IMRRP1 or IMRRP1b.

Antibodies specific for IMRRP1 or IMRRP1b may be polynucleotiderated using methods that are well known in the art. Such antibodies may include, but are not limited to, polyclonal, monoclonal, chimeric, single chain, Fab fragments, and fragments produced by a Fab expression library. Neutralizing antibodies, (i.e., those which inhibit dimer formation) are especially preferred for therapeutic use.

For the production of antibodies, various hosts including goats, rabbits, rats, mice, humans, and others, may be immunized by injection with or any fragment or oligopeptide of IMRRP1 or IMRRP1b which has immunogenic properties. Depending on the host species, various adjuvants may be used to increase immunological response. Such adjuvants include, but are not limited to, Ribi adjuvant R700 (Ribi, Hamilton, Mont.), incomplete Freund's adjuvant, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacillus Calmette Guerin) and Corynebacterium parvumn are especially preferable.

It is preferred that the peptides, fragments, or oligopeptides used to induce antibodies to IMRRP1 or IMRRP1b have an amino acid sequence consisting of at least five amino acids, and more preferably at least 10 amino acids. It is also preferable that they are identical to a portion of the amino acid sequence of the natural protein, and they may contain the entire amino acid sequence of a small, naturally occurring molecule. The peptides, fragments or oligopeptides may comprise a single epitope or antigenic determinant or multiple epitopes. Short stretches of IMRRP1 or IMRRP1b amino acids may be fused with those of another protein such as keyhole limpet hemocyanin and antibody produced against the chimeric molecule.

Monoclonal antibodies to IMRRP1 or IMRRP1b may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42, Cote, R. J. et al. (1983) Proc. Natl. Acad. Sci. 80:2026-2030; Cole, S. P. et al. (1984) Mol. Cell Biol. 62:109-120).

In addition, techniques developed for the production of “chimeric antibodies,” the splicing of mouse antibody polynucleotides to human antibody polynucleotides to obtain a molecule with appropriate antigen specificity and biological activity can be used (Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce IMRRP1- or IMRRP1b-specific single chain antibodies. Antibodies with related specificity, but of distinct idiotypic composition, may be polynucleotiderated by chain shuffling from random combinatorial immunoglobulin libraries (Burton D. R. (1991) Proc. Natl. Acad. Sci. 88:11120-3).

Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments which contain specific binding sites for IMRRP1 or IMRRP1b may also be polynucleotiderated. For example, such fragments include, but are not limited to, the F(ab′)2 fragments which can be produced by pepsin digestion of the antibody molecule and the Fab fragments which can be polynucleotiderated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 254.1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between IMRRP1 or IMRRP1b and their specific antibody. A two-site, monoclonal-based immunoassay utilizing monoclonal antibodies reactive to two non-interfering IMRRP1 or IMRRP1b epitopes is preferred, but a competitive binding assay may also be employed (Maddox, supra).

In another embodiment of the invention, the polynucleotides encoding IMRRP1 or IMRRP1b or any fragment thereof or antisense molecules, may be used for therapeutic purposes. In one aspect, antisense to the polynucleotide encoding IMRRP1 or IMRRP1b may be used in situations in which it would be desirable to block the transcription of the mRNA. In particular, cells may be transformed with sequences complementary to polynucleotides encoding IMRRP1 or IMRRP1b. Thus, antisense molecules may be used to modulate IMRRP1 or IMRRP1b activity, or to achieve regulation of polynucleotide function. Such technology is well known in the art, and sense or antisense oligomers or larger fragments, can be designed from various locations along the coding or control regions of sequences encoding IMRRP1 or IMRRP1b.

Expression vectors derived from retroviruses, adenovirus, herpes or vaccinia viruses, or from various bacterial plasmids may be used for delivery of nucleotide sequences to the targeted organ, tissue or cell population. Methods which are well known to those skilled in the art can be used to construct recombinant vectors which will express antisense molecules complementary to the polynucleotides of the polynucleotides encoding IMRRP1 or IMRRP1b. These techniques are described both in Sambrook et al. (supra) and in Ausubel et al. (supra).

Genes encoding IMRRP1 or IMRRP1b can be turned off by transforming a cell or tissue with expression vectors which express high levels of a polynucleotide or fragment thereof which encodes IMRRP1 or IMRRP1b. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of polynucleotide expression can be obtained by designing antisense molecules, DNA, RNA, or PNA, to the control regions of the polynucleotides encoding IMRRP1 or IMRRP1b, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. L. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples which may be used include engineered hammerhead motif ribozyme molecules that can specifically and efficiently catalyze endonucleolytic cleavage of sequences encoding IMRRP1 or IMRRP1b.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target polynucleotide containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be polynucleotiderated by in vitro and in vivo transcription of DNA sequences encoding IMRRP1 or IMRRP1b. Such DNA sequences may be incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient as disclosed in U.S. Pat. Nos. 5,399,493 and 5,437,994. Delivery by transfection and by liposome injections may be achieved using methods which are well known in the art.

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of IMRRP1 or IMRRP1b, antibodies to IMRRP1 or IMRRP1b, mimetics, agonists, antagonists, or inhibitors of IMRRP1 or IMRRP1b. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions utilized in this invention may be administered by any number of routes including, but not limited to, oral, intravenous, intramuscular, intraarterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Mack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethylcellulose, or sodium carboxymethylcellulose; gums including arabic and tragacanth, and proteins such as gelatin and collagen. If desired, disintegrating or solubilizing agents may be added, such as the cross-linked polyvinyl pyrrohdone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are polynucleotiderally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of IMRRP1 or IMRRP1b, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example IMRRP1 or IMRRP1b or fragments thereof antibodies of IMRRP1 or IMRRP1b, agonists, antagonists or inhibitors of IMRRP1 or IMRRP 1b which ameliorates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, polynucleotideral health of the subject age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 microgram, up to a total dose of about 1 g, depending upon the route of administration. Guidance as to particular dosages and methods of delivery is provided in the literature and polynucleotiderally available to practitioners in the art. In one embodiment, dosages of IMRRP1 or IMRRP1b or fragment thereof from about 1 ng/kg/day to about 10 mg/kg/day, and preferably from about 500 ug/kg/day to about 5 mg/kg/day are expected to induce a biological effect. Those skilled in the art will employ different formulations for nucleotides than for proteins or their inhibitors. Similarly, delivery of polynucleotides or polypeptides will be specific to particular cells, conditions, locations, etc.

In another embodiment, antibodies which specifically bind IMRRP1 or IMRRP1b may be used for the diagnosis of conditions or diseases characterized by expression of IMRRP1 or IMRRP1b, or in assays to monitor patients being treated with IMRRP1 or IMRRP1b, agonists, antagonists or inhibitors. The antibodies useful for diagnostic purposes may be prepared in the same manner as those described above for therapeutics. Diagnostic assays for IMRRP1 or IMRRP1b include methods which utilize the antibody and a label to detect it in human body fluids or extracts of cells or tissues. The antibodies may be used with or without modification, and may be labeled by joining them, either covalently or non-covalently, with a reporter molecule. A wide variety of reporter molecules which are known in the art may be used, several of which are described above.

A variety of protocols including ELISA, RIA, and FACS for measuring IMRRP1 or IMRRP1b are known in the art and provide a basis for diagnosing altered or abnormal levels of IMRRP1 or IMRRP1b expression. Normal or standard values for IMRRP1 or IMRRP1b expression are established by combining body fluids or cell extracts taken from normal mammalian subjects, preferably human, with antibody to IMRRP1 or IMRRP1b under conditions suitable for complex formation. The amount of standard complex formation may be quantified by various methods, but preferably by photometric means. Quantities of IMRRP1 or IMRRP1b expressed in subject samples, control and disease from biopsied tissues are compared with the standard values. Deviation between standard and subject values establishes the parameters for diagnosing disease.

In another embodiment of the invention, the polynucleotides encoding IMRRP1 or IMRRP1b may be used for diagnostic purposes. The polynucleotides which may be used include oligonucleotide sequences, antisense RNA and DNA molecules, and PNAs. The polynucleotides may be used to detect and quantitate polynucleotide expression in biopsied tissues in which expression of IMRRP1 or IMRRP1b may be correlated with disease. The diagnostic assay may be used to distinguish between absence, presence, and excess expression of IMRRP1 or IMRRP1b, and to monitor regulation of IMRRP1 or IMRRP1b levels during therapeutic intervention.

In one aspect, hybridization with PCR probes which are capable of detecting polynucleotide sequences, including genomic sequences, encoding IMRRP1 or IMRRP1b or closely related molecules, may be used to identify nucleic acid sequences which encode IMRRP1 or IMRRP1b. The specificity of the probe, whether it is made from a highly specific region, e.g., 10 unique nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding IMRRP1 or IMRRP1b, alleles, or related sequences.

Probes may also be used for the detection of related sequences, and should preferably contain at least 50% of the nucleotides from any of the IMRRP1 or IMRRP1b encoding sequences. The hybridization probes of the subject invention may be DNA or RNA and derived from the nucleotide sequence of SEQ ID NOS: 1 or 2 or from genomic sequence including promoter, enhancer elements, and introns of the naturally occurring IMRRP1 or IMRRP1b polynucleotides.

Means for producing specific hybridization probes for DNAs encoding IMRRP1 or IMRRP1b include the cloning of nucleic acid sequences encoding IMRRP1 or IMRRP1b or derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as 32P or 35S, or enzymatic labels, such as alkaline phosphatase coupled to the probe via avidin/biotin coupling systems, and the like.

Polynucleotide sequences encoding IMRRP1 or IMRRP1b may be used for the diagnosis of disorders associated with expression of IMRRP1 and IMRRP1b. Examples of such disorders or conditions include regulation of blood pressure, hypertension, induction of feeding, stimulation of firing of locus coeruleus neurons, and stimulation of insulin release, as well as the aberrant induction of the expression of glial fibrillary acidic protein independent of the action of alpha-2 adrenoceptors, dysphoric premenstrual syndrome, neurodepolynucleotiderative disorders such as Alzheimer's disease, opiate addiction, monoamine turnover and therefore nociception, ageing, mood and stroke, salivary disorders and developmental disorders. The polynucleotide sequences encoding IMRRP1 or IMRRP1b may be used in Southern or northern analysis, dot blot, or other membrane-based technologies; in PCR technologies; or in dip stick, pin, ELISA or chip assays utilizing fluids or tissues from patient biopsies to detect altered IMRRP1 or IMRRP1b expression. Such qualitative or quantitative methods are well known in the art.

The nucleotide sequences encoding IMRRP1 or IMRRP1b may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable control sample, the nucleotide sequences have hybridized with nucleotide sequences in the sample, and the presence of altered levels of nucleotide sequences encoding IMRRP1 or IMRRP1b in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of IMRRP1 or IMRRP1b, a normal or standard profile for expression is established. This may be accomplished by combining body fluids or cell extracts taken from normal subjects, either animal or human, with a sequence, or a fragment thereof, which encodes IMRRP1 or IMRRP1b, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

Additional diagnostic uses for oligonucleotides designed from the sequences encoding IMRRP1 or IMRRP1b may involve the use of PCR. Such oligomers may be chemically synthesized, polynucleotiderated enzymatically, or produced from a recombinant source. Oligomers will preferably consist of two nucleotide sequences, one with sense orientation (5′≧3′) and another with antisense (3′≧5′), employed under optimized conditions for identification of a specific polynucleotide or condition. The same two oligomers, nested sets of oligomers, or even a depolynucleotiderate pool of oligomers may be employed under less stringent conditions for detection and/or quantitation of closely related DNA or RNA sequences.

Methods which may also be used to quantitate the expression of IMRRP1 or IMRRP1b include radiolabeling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 229-236). The speed of quantitation of multiple samples may be accelerated by running the assay in an ELISA format where the oligomer of interest is presented in various dilutions and a spectrophotometric or calorimetric response gives rapid quantitation.

In another embodiment of the invention, the nucleic acid sequences which encode IMRRP1 or IMRRP1b may also be used to polynucleotiderate hybridization probes which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154.

FISH (as described in Verma et al. (1988) Human Chromosomes: A Manual of Basic Techniques Pergamon Press, New York, N.Y.) may be correlated with other physical chromosome mapping techniques and polynucleotidetic map data. Examples of polynucleotidetic map data can be found in the 1994 Genome Issue of Science (265:1981f). Correlation between the location of the polynucleotide encoding IMRRP1 or IMRRP1b on a physical chromosomal map and a specific disease, or predisposition to a specific disease, may help delimit the region of DNA associated with that polynucleotidetic disease. The nucleotide sequences of the subject invention may be used to detect differences in polynucleotide sequences between normal, carrier, or affected individuals.

In situ hybridization of chromosomal preparations and physical mapping techniques such as linkage analysis using established chromosomal markers may be used for extending polynucleotidetic maps. Often the placement of a polynucleotide on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease polynucleotides using positional cloning or other polynucleotide discovery techniques. Once the disease or syndrome has been crudely localized by polynucleotidetic linkage to a particular genomic region, for example, AT to 11q22-23 (Gatti, R. A. et al. (1988) Nature 336:577-580), any sequences mapping to that area may represent associated or regulatory polynucleotides for further investigation. The nucleotide sequence of the subject invention may also be used to detect differences in the chromosomal location due to translocation, inversion, etc. among normal, carrier, or affected individuals.

In another embodiment of the invention, IMRRP1 or IMRRP1b, their catalytic or immunogenic fragments or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes, between IMRRP1 or IMRRP1b and the agent being tested, may be measured.

Another technique for drug screening which may be used provides for high throughput screening of compounds having suitable binding affinity to the protein of interest as described in published PCT application W084/03564. In this method, as applied to IMRRP1 or IMRRP1b, large numbers of different small test compounds are synthesized on a solid substrate, such as plastic pins or some other surface. The test compounds are contacted with IMRRP1 or IMRRP1b or fragments thereof, and washed. Bound IMRRP1 or IMRRP1b are then detected by methods well known in the art. Purified IMRRP1 or IMRRP1b can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding IMRRP1 or IMRRP1b specifically compete with a test compound for binding IMRRP1 or IMRRP1b. In this manner, the antibodies can be used to detect the presence of any peptide which shares one or more antigenic determinants with IMRRP1 or IMRRP1b.

In additional embodiments, the nucleotide sequences which encode IMRRP1 or IMRRP1b may be used in any molecular biology techniques that have yet to be developed, provided the new techniques rely on properties of nucleotide sequences that are currently known, including, but not limited to, such properties as the triplet polynucleotidetic code and specific base pair interactions.

The examples below are provided to illustrate the subject invention and are not included for the purpose of limiting the invention. All publications and patents mentioned in the specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### EXAMPLES

### Example 1

Method of Isolation of cDNA Encoding IMRRP1 or IMRRP1b

Human imidazoline receptor protein sequence was used as a probe to search the Incyte and public domain EST databases. The search program used was gapped BLAST (Altschul et al., 1997). The top EST hits from the BLAST results were searched back against the non-redundant protein and patent sequence databases. From this analysis, ESTs encoding a potential novel imidazoline receptor was identified based on sequence homology. The Incyte EST (CloneID: 2499870) was selected as a potential novel imidazoline receptor candidate for subsequent analysis.

A PCR primer pair, designed from the DNA sequence of Incyte clone-2499870 was used to amplify a piece of DNA from the clone in which the anti-sense strand of the amplified fragment was biotinylated on the 5′ end. This biotinylated piece of double stranded DNA was denatured and incubated with a mixture of single-stranded covalently closed circular cDNA libraries which contain DNA corresponding to the sense strand. The cDNA libraries were total brain tissue libraries obtained from Gibco Life Technologies. Hybrids between the biotinylated DNA and the circular cDNA were captured on streptavidin magnetic beads. Upon thermal release of the cDNA from the biotinylated DNA, the single stranded cDNA was converted into double strands using a primer homologous to a sequence on the cDNA cloning vector. The double stranded cDNA was introduced into E. coli by electroporation and the resulting colonies were screen by PCR, using the original primer pair, to identify the proper cDNA clones. One clone named FL1-18 was sequenced on both strands (FIG. 1).

### Example 2

Cellular and Tissue Distribution of IMRRP1

The same PCR primer used in the cloning of imidazoline receptor IMRRP1 used to measure the steady state levels of mRNA by quantitative PCR. Briefly, first strand cDNA was made from commercially available mRNA. The relative amount of cDNA used in each assay was determined by performing a parallel experiment using a primer pair for a polynucleotide expressed in equal amounts in all tissues, cyclophilin. The cyclophilin primer pair detected small variations in the amount of cDNA in each sample and these data were used for normalization of the data obtained with the primer pair for IMRRP1. The PCR data was converted into a relative assessment of the difference in transcript abundance amongst the tissues tested and the data is presented in FIG. 7.

### Example 3

Labeling and Use of Hybridization Probes

Hybridization probes derived from SEQ ID NOS: 1 or 2 are employed to screen cDNAs, genomic DNAs, or mRNAs. Although the labeling of oligonucleotides, consisting of about 20 base-pairs, is specifically described, essentially the same procedure is used with larger cDNA fragments. Oligonucleotides are designed using state-of-the-art software such as OLIGO 4.06 (National Biosciences), labeled by combining 50 μmol of each oligomer and 250 μCi of [γ-³²P] adenosine triphosphate (Amersham) and T4 polynucleotide kinase (DuPont NEN, Boston, Mass.). The labeled oligonucleotides are substantially purified with SEPHADEX G-25 superfine resin column (Pharmacia & Upjohn). A portion containing about 10⁷ counts per minute of each of the sense and antisense oligonucleotides is used in a typical membrane based hybridization analysis of human genomic DNA digesed with one of the following endonucleases (Ase I, Bgl II, Eco RI, Pst I, Xba 1, or Pvu II: DuPont NEN).

The DNA from each digest is fractionated on a 0.7 percent agarose gel and transferred to nylon membranes (Nytran Plus, Schleicher & Schuell, Durham, N.H.). Hybridization is carried out for 16 hours at 40° C. To remove nonspecific signals, blots are sequentially washed at room temperature under increasingly stringent conditions up to 0.1×saline sodium citrate and 0.5% sodium dodecyl sulfate. After XOMATAR film (Kodak, Rochester, N.Y.) is exposed to the blots in a Phosphoimager cassette (Molecular Dynamics, Sunnyvale, Calif.) for several hours, hybridization patterns are compared visually.

### Example 4

Antisense Molecules

Antisense molecules or nucleic acid sequence complementary to the IMRRP1 or IMRRP1b encoding sequences, or any part thereof, is used to inhibit in vivo or in vitro expression of naturally occurring IMRRP1 or IMRRP1b. Although use of antisense oligonucleotides, comprising about 20 base-pairs, is specifically described, essentially the same procedure is used with larger cDNA fragments. An oligonucleotide based on the coding sequences of IL-17R, as shown in FIGS. 1 and 2 is used to inhibit expression of naturally occurring IMRRP1 or IMRRP1b. The complementary oligonucleotide is designed from the unique 5′ sequence as shown in FIG. 1 or 2 and used either to inhibit transcription by preventing promoter binding to the upstream nontranslated sequence or translation of an IMRRP1 or IMRRP1b encoding transcript by preventing the ribosome from binding. Using an appropriate portion of the signal and 5′ sequence of SEQ ID NOS: 1 or 2 an effective antisense oligonucleotide includes any 15-20 nucleotides spanning the region which translates into the signal or 5′ coding sequence of the polypeptide as shown in FIGS. 1 and 2.

### Example 5

Production of IMRRP1 or IMRRP1b Specific Antibodies

IMRRP1 or IMRRP1b that is substantially purified using PAGE electrophoresis (Sambrook, supra), or other purification techniques, is used to immunize rabbits and to produce antibodies using standard protocols. The amino acid sequence from SEQ ID NOS: 3 or 4 is analyzed using DNASTAR software (DNASTAR Inc.) to determine regions of high immunogenicity and a corresponding oligopolypepide is synthesized and used to raise antibodies by means known to those of skill in the art. Selection of appropriate epitopes, such as those near the C-terminus or in hydrophilic regions, is described by Ausubel et al. (supra) and others.

Typically, the oligopeptides are 15 residues in length, synthesized using an Applied Biosystems Peptide Synthesizer Model 431A using fmoc-chemistry, and coupled to keyhole limpet hemacyanin (KLH, Sigma, St. Lousi, Mo.) by reaction with N-maleimidobenzoyl-N-hydroxysuccinimide ester (MBS; Ausubel et al., supra). Rabbits are immunized with the oligopeptide-KLH complex in complete Freund's adjuvant. The resulting antisera are tested for antipeptide activity, for example, by binding the rabbit antisera, washing, and reacting with radioiodinated, goat and anti-rabbit IgG.

### Example 6

Purification of Naturally Occurring IMRRP1 or IMRRP1b Using Specific Antibodies

Naturally occurring or recombinant IMRRP1 or IMRRP1b is substantially purified by immunoaffinity chromatography using antibodies specific for IMRRP1 or IMRRP1b. An immunoaffinity column is constructed by covalently coupling IMRRP1 or IMRRP1b specific antibody to an activated chromatographic resin, such as CNRr-activated SEPHAROSE (Pharmacia & Upjohn). After the coupling, the resin is blocked and washed according to the manufacturer's instructions.

Media containing IMRRP1 or IMRRP1b is passed over the immunoaffinity column, and the column is washed under conditions that allow the preferential absorbance of IMRRP1 or IMRRP1b (e.g., high ionic strength buffers in the presence of detergent). The column is eluted under conditions that disrupt antibody—IMRRP1 or IMRRP1b binding (e.g., buffer of pH 2-3 or a high concentration of a chaotrope, such as urea or thiocyanate ion), and IMRRP1 or IMRRP1b is collected.

### Example 7

Identification of Molecules which Interact with IMRRP1 or IMRRP1b

IMRRP1 or IMRRP1b or biologically active fragments thereof are labeled with ¹²⁵I Bolton-Hunter reagent (Bolton et al. (1973) Biochem. J., 133:529). Candidate molecules previously arrayed in the wells of a multi-well plate are incubated with the labeled IMRRP1 or IMRRP1b, washed and any wells with labeled IMRRP1 or IMRRP1b complex are assayed. Data obtained using different concentrations of IMRRP1 or IMRRP1b are used to calculate values for the number, affinity, and associate of IMRRP1 or IMRRP1b with the candidate molecules.

### Example 8

Expression Profiling of IMMRP1

Expression profiling in 12 tissue RNA samples was carried out to show the overall pattern of polynucleotide expression in the body. The same PCR primer pair, shown below as Incyte-2499870, that was used to identify IMMRP1 cDNA clones was used to measure the steady state levels of mRNA by quantitative PCR.

| | |
|---|---|
| INCYTE-2499870-s | |
| GCTGGAGACCCTGATTTGCA | (SEQ ID NO:5) |
| INCYTE-2499870-ab | |
| bTGGACTTGATTGTGGCTTAGGTT | (SEQ ID NO:6) |

First strand cDNA was made from commercially available mRNA (Clontech, Stratapolynucleotide, and Life Technologies) and subjected to real time quantitative PCR using a PE 5700 instrument (Applied Biosystems, Foster City, Calif.) which detects the amount of DNA amplified during each cycle by the fluorescent output of SYBR green, a DNA binding dye specific for double strands. The specificity of the primer pair for its target is verified by performing a thermal denaturation profile at the end of the run which gives an indication of the number of different DNA sequences present by determining melting Tm. In the case of the FGFR1ΔCP primer pair, only one DNA fragment was detected having a homopolynucleotideous melting point. Contributions of contaminating genomic DNA to the assessment of tissue abundance is controlled for by performing the PCR with first strand made with and without reverse transcriptase. In all cases, the contribution of material amplified in the no reverse transcriptase controls was negligible.

Small variations in the amount of cDNA used in each tube was determined by performing a parallel experiment using a primer pair for cyclophilin, a polynucleotide expressed in equal amounts in all tissues. These data were used to normalize the data obtained with the IMMRP1 primer pair. The PCR data was converted into a relative assessment of the difference in transcript abundance amongst the tissues tested and the data are presented in bar graph form in FIG. 8. Transcripts corresponding to IMMRP1 were found in all the additional RNAs tested with the highest amount present in the testis (like that of the first panel tested). Relatively high expression was also observed in the salivary gland and the fetal brain.

The quantitative PCT was performed by determining the number of reactions and amount of mix needed. All samples were run in triplicate, so each sample tube need 3.5 reactions worth of mix. This is determined by the following formula: 2×#tissue samples+1 no template control+1 for pipetting error.

The reaction mixture was prepared as follows.

| Components | vol/rxn | |
|---|---|---|
| 2X SybrGreen Master Mix | 25 | microliters |
| water | 23.5 | microliters |
| primer mix (10 μM ea.) | 0.5 | microliters |
| cDNA (2.5 ng/μL) | 1 | microliter |

An aliquot 171.5 μL of mix was added to each to sample tubes followed by the addition of 1 μL of cDNA. Each sample tube was mixed gently and spun down. An aliquot of 3×50 μL was added to an optical plate and analyzed.

### Example 9

Complementary Polynucleotides

Antisense molecules or nucleic acid sequences complementary to the IMRRP1 or IMRRP1b protein-encoding sequence, or any part thereof, is used to decrease or to inhibit the expression of naturally occurring IMRRP1 or IMRRP1b. Although the use of antisense or complementary oligonucleotides comprising about 15 to 35 base-pairs is described, essentially the same procedure is used with smaller or larger nucleic acid sequence fragments. An oligonucleotide based on the coding sequence of IMRRP1 or IMRRP1b protein, as shown in FIGS. 1-2, or as depicted in SEQ ID NO: 1 or SEQ ID NO:2 for example, is used to inhibit expression of naturally occurring IMRRP1 and/or IMRRP1b. The complementary oligonucleotide is typically designed from the most unique 5′ sequence and is used either to inhibit transcription by preventing promoter binding to the coding sequence, or to inhibit translation by preventing the ribosome from binding to the IMRRP1 and/or IMRRP1b protein-encoding transcript, among others. However, other regions may also be targeted.

Using an appropriate portion of the signal and 5′ sequence of SEQ ID NO: 1 or SEQ ID NO:2, an effective antisense oligonucleotide includes any of about 15-35 nucleotides spanning the region which translates into the signal or 5′ coding sequence, among other regions, of the polypeptide as shown in FIGS. 3-4 (SEQ ID NO:3 or SEQ ID NO:4). Appropriate oligonucleotides are designed using OLIGO 4.06 software and the IMRRP1 and/or IMRRP1b protein coding sequence (SEQ ID NO: 1or SEQ ID NO:2). Preferred oligonucleotides are deoxynucleotide, or chimeric deoxynucleotide/ribonucleotide based and are provided below. The oligonucleotides were synthesized using chemistry essentially as described in U.S. Pat. No. 5,849,902; which is hereby incorporated herein by reference in its entirety.

| | |
|---|---|
| ID# | Sequence |

| | | |
|---|---|---|
| 13606 | CCCAGGUGCAGCUCAAAUACGUGGU | (SEQ ID NO:14) |
| 13607 | CAUUCUUGGCACCAAAUGCAGGCGA | (SEQ ID NO:15) |
| 13608 | AUUCCUCAGCUGCUCUAGGCCAUGC | (SEQ ID NO:16) |
| 13609 | GUCCUUCCAGCAGGUUGUAUGCCAA | (SEQ ID NO:17) |
| 13610 | CCUUGCCAUCGAGAAGGAAGCCAGU | (SEQ ID NO:18) |

The IMRRP1 and/or IMRRP1B polypeptide has been shown to be involved in the regulation of mammalian cell cycle pathways. Subjecting cells with an effective amount of a pool of all five of the above antisense oligoncleotides resulted in a significant decrease in p21 expression/activity providing convincing evidence that IMRRP1 and/or IMRRP1B at least regulates the activity and/or expression of p21 either directly, or indirectly. Moreover, the results suggest that IMRRP1 and/or IMRRP1B is involved in the positive/negative regulation of p21 activity and/or expression, either directly or indirectly. The p21 assay used is described below and was based upon the analysis of p21 activity as a downstream marker for proliferative signal transduction events.

### Transfection of Post-Quiescent A549 Cells with AntiSense Oligonucleotides

### Materials Needed

A549 cells maintained in DMEM with high glucose (Gibco-BRL) supplemented with 10% Fetal Bovine Serum, 2 mM L-Glutamine, and IX penicillin/streptomycin.

Opti-MEM (Gibco-BRL)

Lipofectamine 2000 (Invitrogen)

Antisense oligomers (Sequitur)

Polystyrene tubes.

Tissue culture treated plates.

Quiescent cells were prepared as follows:

Day 0: 300, 000 A549 cells were seeded in a T75 tissue culture flask in 10 ml of A549 media (as specified above), and incubated in at 37° C., 5% CO₂ in a humidified incubator for 48 hours.

Day 2: The T75 flasks were rocked to remove any loosely adherent cells, and the A549 growth media removed and replenished with 10 ml of fresh A549 media. The cells were cultured for six days without changing the media to create a quiescent cell population.

Day 8: Quiescent cells were plated in multi-well format and transfected with antisense oligonucleotides.

A549 cells were transfected according to the following:

1. Trypsinize T75 flask containing quiescent population of A549 cells.

2. Count the cells and seed 24-well plates with 60K quiescent A549 cells per well.

3. Allow the cells to adhere to the tissue culture plate (approximately 4 hours).

4. Transfect the cells with antisense and control oligonucleotides according to the following:

a. A 10×stock of lipofectamine 2000 (10 ug/ml is 10×) was prepared, and diluted lipid was allowed to stand at RT for 15 minutes.

Stock solution of lipofectamine 2000 was 1 mg/ml.

10×solution for transfection was 10 ug/mI.

To prepare 10×solution, dilute 10 ul of lipofectamine 2000 stock per 1 ml of Opti-MEM (serum free media).

b. A 10×stock of each oligomer was prepared to be used in the transfection.

Stock solutions of oligomers were at 100 uM in 20 mM HEPES, pH 7.5. 10×concentration of oligomer was 0.25 uM.

To prepare the 10×solutions, dilute 2.5 ul of oligomer per 1 ml of Opti-MEM.

c. Equal volumes of the 10×lipofectamine 2000 stock and the 10×oligomer solutions were mixed well, and incubated for 15 minutes at RT to allow complexation of the oligomer and lipid. The resulting mixture was 5×.

d. After the 15 minute complexation, 4 volumes of full growth media was added to the oligomer/lipid complexes (solution was 1×).

e. The media was aspirated from the cells, and 0.5 ml of the 1×oligomer/lipid complexes added to each well.

f. The cells were incubated for 16-24 hours at 37° C. in a humidified CO₂ incubator.

g. Cell pellets were harvested for RNA isolation and TaqMan analysis of downstream marker polynucleotides.

TaqMan Reactions

Quantitative RT-PCR analysis was performed on total RNA preps that had been treated with DNaseI or poly A selected RNA. The Dnase treatment may be performed using methods known in the art, though preferably using a Qiagen RNeasy kit to purify the RNA samples, wherein DNAse I treatment is performed on the column.

Briefly, a master mix of reagents was prepared according to the following table:

Dnase I Treatment

| Reagent | Per r'xn (in uL) |
|---|---|
| 10x Buffer | 2.5 |
| Dnase I (1 unit/ul @ 1 unit per ug sample) | 2 |
| DEPC H₂O | 0.5 |
| RNA sample @ 0.1 ug/ul | 20 |
| (2-3 ug total) | |
| Total | 25 |

Next, 5 ul of master mix was aliquoted per well of a 96-well PCR reaction plate (PE part #N801-0560). RNA samples were adjusted to 0.1 ug/ul with DEPC treated H₂O (if necessary), and 20 ul was added to the aliquoted master mix for a final reaction volume of 25 ul.

The wells were capped using strip well caps (PE part #N801-0935), placed in a plate, and briefly spun in a plate centrifuge (Beckman) to collect all volume in the bottom of the tubes. Generally, a short spin up to 500 rpm in a Sorvall RT is sufficient

The plates were incubated at 37° C. for 30 mins. Then, an equal volume of 0.1 mM EDTA in 10 mM Tris was added to each well, and heat inactivated at 70° C. for 5 min. The plates were stored at −80° C. upon completion.

RT Reaction

A master mix of reagents was prepared according to the following table:

RT reaction

| | RT | | No RT | |
|---|---|---|---|---|
| Reagent | Per Rx'n (in ul) | | Per Rx'n (in ul) | |
| 10x RT buffer | 5 | | 2.5 | |
| MgCl₂ | 11 | | 5.5 | |
| DNTP mixture | 10 | | 5 | |
| Random Hexamers | 2.5 | | 1.25 | |
| Rnase inhibitors | 1.25 | | 0.625 | |
| RT enzyme | 1.25 | | — | |
| Total RNA 500 ng (100 ng no RT) | 19.0 | max | 10.125 | max |
| DEPC H₂O | — | | — | |
| Total | 50 | uL | 25 | uL |

Samples were adjusted to a concentration so that 500 ng of RNA was added to each RT rx′n (100 ng for the no RT). A maximum of 19 ul can be added to the RT rx′n mixture (10.125 ul for the no RT.) Any remaining volume up to the maximum values was filled with DEPC treated H₂O, so that the total reaction volume was 50 ul (RT) or 25 ul (no RT).

On a 96-well PCR reaction plate (PE part #N801-0560), 37.5 ul of master mix was aliquoted (22.5 ul of no RT master mix), and the RNA sample added for a total reaction volume of 50 ul (25 ul, no RT). Control samples were loaded into two or even three different wells in order to have enough template for polynucleotideration of a standard curve.

The wells were capped using strip well caps (PE part # N801-0935), placed in a plate, and spin briefly in a plate centrifuge (Beckman) to collect all volume in the bottom of the tubes. Generally, a short spin up to 500 rpm in a Sorvall RT is sufficient.

For the RT-PCR reaction, the following thermal profile was used:

25° C. for 10 min

48° C. for 30 min

95° C. for 5 min

4° C. hold (for 1 hour)

Store plate @-20° C. or lower upon completion.

### TaqMan Reaction (Template Comes from RT Plate)

A master mix was prepared according to the following table:

TaqMan reaction (per well)

| Reagent | Per Rx'n (in ul) |
|---|---|
| TaqMan Master Mix | 4.17 |
| 100 uM Probe | .025 |
| (SEQ ID NO:21) | |
| 100 uM Forward primer | .05 |
| (SEQ ID NO:19) | |
| 100 uM Reverse primer | .05 |
| (SEQ ID NO:20) | |
| Template | — |
| DEPC H₂O | 18.21 |
| Total | 22.5 |

The primers used for the RT-PCR reaction is as follows:

### P21 Primer and Probes

| | |
|---|---|
| Forward Primer: | |
| CTGGAGACTCTCAGGGTCGAA | (SEQ ID NO:19) |
| Reverse Primer: | |
| GCGCTTCCAGGACTGCA | (SEQ ID NO:20) |
| TaqMan Probe: | |
| ACAGATTTCTACCACTCCAAACGCCGG | (SEQ ID NO:21) |

Using a Gilson P-10 repeat pipetter, 22.5 ul of master mix was aliquouted per well of a 96-well optical plate. Then, using P-10 pipetter, 2.5 ul of sample was added to individual wells. Generally, RT samples are run in triplicate with each primer/probe set used, and no RT samples are run once and only with one primer/probe set, often graph (or other internal control).

A standard curve is then constructed and loaded onto the plate. The curve has five points plus one no template control (NTC, =DEPC treated H₂O). The curve was made with a high point of 50 ng of sample (twice the amount of RNA in unknowns), and successive samples of 25, 10, 5, and 1 ng. The curve was made from a control sample(s) (see above).

The wells were capped using optical strip well caps (PE part #N801-0935), placed in a plate, and spun in a centrifuge to collect all volume in the bottom of the tubes. Generally, a short spin up to 500 rpm in a Sorvall RT is sufficient.

Plates were loaded onto a PE 5700 sequence detector making sure the plate is aligned properly with the notch in the upper right hand corner. The lid was tightened down and run using the 5700 and 5700 quantitation program and the SYBR probe using the following thermal profile:

50° C. for 2 min

95° C. for 10 min

and the following for 40 cycles:

95° C. for 15 sec

60° C. for 1 min

Change the reaction volume to 25 ul.

Once the reaction was complete, a manual threshold of around 0.1 was set to minimize the background signal. Additional information relative to operation of the GeneAmp 5700 machine may be found in reference to the following manuals: “GeneAmp 5700 Sequence Detection System Operator Training CD”; and the “User's Manual for 5700 Sequence Detection System”; available from Perkin-Elmer and hereby incorporated by reference herein in their entirety.

### Example 10

Complementary Polynucleotides

Antisense molecules or nucleic acid sequences complementary to the IMRRP1 and/or IMRRP1b protein-encoding sequence, or any part thereof, was used to decrease or to inhibit the expression of naturally occurring IMRRP1 and/or IMRRP1b. Although the use of antisense or complementary oligonucleotides comprising about 15 to 35 base-pairs is described, essentially the same procedure is used with smaller or larger nucleic acid sequence fragments. An oligonucleotide based on the coding sequence of IMRRP1 and/or IMRRP1b protein, as shown in FIGS. 1-2, or as depicted in SEQ ID NO: 1 or SEQ ID NO:2 for example, is used to inhibit expression of naturally occurring IMRRP1 and/or IMRRP1b. The complementary oligonucleotide is typically designed from the most unique 5′ sequence and is used either to inhibit transcription by preventing promoter binding to the coding sequence, or to inhibit translation by preventing the ribosome from binding to the IMRRP1 and/or IMRRP1b protein-encoding transcript, among others. However, other regions may also be targeted.

Using an appropriate portion of a 5′ sequence of SEQ ID NO: 1 or SEQ ID NO:2, an effective antisense oligonucleotide includes any of about 15-35 nucleotides spanning the region which translates into the signal or 5′ coding sequence, among other regions, of the polypeptide as shown in FIGS. 3-4 (SEQ ID NO:3 or SEQ ID NO:4). Appropriate oligonucleotides are designed using OLIGO 4.06 software and the IMRRP1 and/or IMRRP1b protein coding sequence (SEQ ID NO: 1 or SEQ ID NO:2). Preferred oligonucleotides are deoxynucleotide, or chimeric deoxynucleotide/ribonucleotide based and are provided below. The oligonucleotides were synthesized using chemistry essentially as described in U.S. Pat. No. 5,849,902; which is hereby incorporated herein by reference in its entirety.

| | |
|---|---|
| ID# | Sequence |

| | | |
|---|---|---|
| 13606 | CCCAGGUGCAGCUCAAAUACGUGGU | (SEQ ID NO:14) |
| 13607 | CAUUCUUGGCACCAAAUGCAGGCGA | (SEQ ID NO:15) |
| 13608 | AUUCCUCAGCUGCUCUAGGCCAUGC | (SEQ ID NO:16) |
| 13609 | GUCCUUCCAGCAGGUUGUAUGCCAA | (SEQ ID NO:17) |
| 13610 | CCUUGCCAUCGAGAAGGAAGCCAGU | (SEQ ID NO:18) |

The IMRRP1 and/or IMRRP1b polypeptide has been shown to be involved in the regulation of mammalian NF-κB and apoptosis pathways. Subjecting cells with an effective amount of a pool of all five of the above antisense oligoncleotides resulted in a significant increase in IκBα expression/activity providing convincing evidence that IMRRP1 and/or IMRRP1b at least regulates the activity and/or expression of IκBα either directly, or indirectly. Moreover, the results suggest that IMRRP1 and/or IMRRP1b is involved in the positive/negative regulation of NF-κB/IκBα activity and/or expression, either directly or indirectly. The IκBα assay used is described below and was based upon the analysis of IκBα activity as a downstream marker for proliferative signal transduction events.

### Transfection of Post-Quiescent A549 Cells with AntiSense Oligonucleotides

### Materials Needed

A549 cells maintained in DMEM with high glucose (Gibco-BRL) supplemented with 10% Fetal Bovine Serum, 2 mM L-Glutamine, and 1×penicillin/streptomycin.

Opti-MEM (Gibco-BRL)

Lipofectamine 2000 (Invitrogen)

Antisense oligomers (Sequitur)

Polystyrene tubes.

Tissue culture treated plates.

Quiescent cells were prepared as follows:

Day 0: 300, 000 A549 cells were seeded in a T75 tissue culture flask in 10 ml of A549 media (as specified above), and incubated in at 37° C., 5% CO₂ in a humidified incubator for 48 hours.

Day 2: The T75 flasks were rocked to remove any loosely adherent cells, and the A549 growth media removed and replenished with 10 ml of fresh A549 media. The cells were cultured for six days without changing the media to create a quiescent cell population.

Day 8: Quiescent cells were plated in multi-well format and transfected with antisense oligonucleotides.

A549 cells were transfected according to the following:

1. Trypsinize T75 flask containing quiescent population of A549 cells.

2. Count the cells and seed 24-well plates with 60K quiescent A549 cells per well.

3. Allow the cells to adhere to the tissue culture plate (approximately 4 hours).

4. Transfect the cells with antisense and control oligonucleotides according to the following:

a. A 10×stock of lipofectamine 2000 (10 ug/ml is 10×) was prepared, and diluted lipid was allowed to stand at RT for 15 minutes.

Stock solution of lipofectamine 2000 was 1 mg/ml.

10×solution for transfection was 10 ug/ml.

To prepare 1×solution, dilute 10 ul of lipofectamine 2000 stock per 1 ml of Opti-MEM (serum free media).

b. A 10×stock of each oligomer was prepared to be used in the transfection.

Stock solutions of oligomers were at 100 uM in 20 mM HEPES, pH 7.5.

10×concentration of oligomer was 0.25 uM.

To prepare the 10×solutions, dilute 2.5 ul of oligomer per 1 ml of Opti-MEM.

c. Equal volumes of the 10×lipofectamine 2000 stock and the 10×oligomer solutions were mixed well, and incubated for 15 minutes at RT to allow complexation of the oligomer and lipid. The resulting mixture was 5×.

d. After the 15 minute complexation, 4 volumes of full growth media was added to the oligomer/lipid complexes (solution was 1×).

e. The media was aspirated from the cells, and 0.5 ml of the 1×oligomer/lipid complexes added to each well.

f. The cells were incubated for 16-24 hours at 37° C. in a humidified CO₂ incubator.

g. Cell pellets were harvested for RNA isolation and TaqMan analysis of downstream marker polynucleotides.

### TaqMan Reactions

Quantitative RT-PCR analysis was performed on total RNA preps that had been treated with DNaseI or poly A selected RNA. The Dnase treatment may be performed using methods known in the art, though preferably using a Qiagen RNeasy kit to purify the RNA samples, wherein DNAse I treatment is performed on the column.

Briefly, a master mix of reagents was prepared according to the following table:

Dnase I Treatment

| Reagent | Per r'xn (in uL) |
|---|---|
| 10x Buffer | 2.5 |
| Dnase I (1 unit/ul @ 1 unit per ug sample) | 2 |
| DEPC H₂O | 0.5 |
| RNA sample @ 0.1 ug/ul | 20 |
| (2-3 ug total) | |
| Total | 25 |

Next, 5 ul of master mix was aliquoted per well of a 96-well PCR reaction plate (PE part #N801-0560). RNA samples were adjusted to 0.1 ug/ul with DEPC treated H₂O (if necessary), and 20 ul was added to the aliquoted master mix for a final reaction volume of 25 ul.

The wells were capped using strip well caps (PE part #N801-0935), placed in a plate, and briefly spun in a plate centrifuge (Beckman) to collect all volume in the bottom of the tubes. Generally, a short spin up to 500 rpm in a Sorvall RT is sufficient The plates were incubated at 37° C. for 30 mins. Then, an equal volume of 0.1 mM EDTA in 10 mM Tris was added to each well, and heat inactivated at 70° C. for 5 min. The plates were stored at −80° C. upon completion.

### RT Reaction

A master mix of reagents was prepared according to the following table:

RT reaction

| | RT | | No RT | |
|---|---|---|---|---|
| Reagent | Per Rx'n (in ul) | | Per Rx'n (in ul) | |
| 10x RT buffer | 5 | | 2.5 | |
| MgCl₂ | 11 | | 5.5 | |
| DNTP mixture | 10 | | 5 | |
| Random Hexamers | 2.5 | | 1.25 | |
| Rnase inhibitors | 1.25 | | 0.625 | |
| RT enzyme | 1.25 | | — | |
| Total RNA 500 ng (100 ng no RT) | 19.0 | max | 10.125 | max |
| DEPC H₂O | — | | — | |
| Total | 50 | uL | 25 | uL |

Samples were adjusted to a concentration so that 500 ng of RNA was added to each RT rx′n (100 ng for the no RT). A maximum of 19 ul can be added to the RT rx′n mixture (10.125 ul for the no RT.) Any remaining volume up to the maximum values was filled with DEPC treated H₂O, so that the total reaction volume was 50 ul (RT) or 25 ul (no RT).

On a 96-well PCR reaction plate (PE part #N801-0560), 37.5 ul of master mix was aliquoted (22.5 ul of no RT master mix), and the RNA sample added for a total reaction volume of 50 ul (25 ul, no RT). Control samples were loaded into two or even three different wells in order to have enough template for polynucleotideration of a standard curve.

The wells were capped using strip well caps (PE part #N801-0935), placed in a plate, and spin briefly in a centrifuge to collect all volume in the bottom of the tubes. Generally, a short spin up to 500 rpm in a Sorvall RT is sufficient.

For the RT-PCR reaction, the following thermal profile was used:

25° C. for 10 min

48° C. for 30 min

95° C. for 5 min

4° C. hold (for 1 hour)

Store plate @-20° C. or lower upon completion.

### TaqMan Reaction (Template Comes from RT Plate)

A master mix was prepared according to the following table:

TaqMan reaction (per well)

| Reagent | Per Rx'n (in ul) |
|---|---|
| TaqMan Master Mix | 4.17 |
| 100 uM Probe | .025 |
| (SEQ ID NO:24) | |
| 100 uM Forward primer | .05 |
| (SEQ ID NO:22) | |
| 100 uM Reverse primer | .05 |
| (SEQ ID NO:23) | |
| Template | — |
| DEPC H₂O | 18.21 |
| Total | 22.5 |

The primers used for the RT-PCR reaction is as follows:

### IκBα Primer and Probes

| | |
|---|---|
| Forward Primer: | GAGGATGAGGAGAGCTATGACACA |
| | (SEQ ID NO:22) |
| Reverse Primer: | CCCTTTGCACTCATAACGTCAG |
| | (SEQ ID NO:23) |
| TaqMan Probe: | AAACACACAGTCATCATAGGGCAGCTCGT |
| | (SEQ ID NO:24) |

Using a Gilson P-10 repeat pipetter, 22.5 ul of master mix was aliquouted per well of a 96-well optical plate. Then, using P-10 pipetter, 2.5 ul of sample was added to individual wells. Generally, RT samples are run in triplicate with each primer/probe set used, and no RT samples are run once and only with one primer/probe set, often gapdh (or other internal control).

A standard curve is then constructed and loaded onto the plate. The curve has five points plus one no template control (NTC, =DEPC treated H₂O). The curve was made with a high point of 50 ng of sample (twice the amount of RNA in unknowns), and successive samples of 25, 10, 5, and 1 ng. The curve was made from a control sample(s) (see above).

The wells were capped using optical strip well caps (PE part #N801-0935), placed in a plate, and spun in a centrifuge to collect all volume in the bottom of the tubes. Generally, a short spin up to 500 rpm in a Sorvall RT is sufficient.

Plates were loaded onto a PE 5700 sequence detector making sure the plate is aligned properly with the notch in the upper right hand corner. The lid was tightened down and run using the 5700 and 5700 quantitation program and the SYBR probe using the following thermal profile:

50° C. for 2 min

95° C. for 10 min

and the following for 40 cycles:

95° C. for 15 sec

60° C. for 1 min

Change the reaction volume to 25 ul.

Once the reaction was complete, a manual threshold of around 0.1 was set to minimuze the background signal. Additional information relative to operation of the GeneAmp 5700 machine may be found in reference to the following manuals: “GeneAmp 5700 Sequence Detection System Operator Training CD”; and the “User's Manual for 5700 Sequence Detection System”; available from Perkin-Elmer and hereby incorporated by reference herein in their entirety.

### Example 11

Method of Assessing the Expression Profile of the Novel Immidazoline Receptorpolypeptides of the Present Invention in a Variety of Cancer Cell Lines

RNA quantification may, be performed using the Taqman® real-time-PCR fluorogenic assay. The Taqman® assay is one of the most precise methods for assaying the concentration of nucleic acid templates.

All cell lines were grown using standard conditions: RPMI 1640 supplemented with 10% fetal bovine serum, 100 IU/ml penicillin, 100 mg/ml streptomycin, and 2 mM L-glutamine, 10 mM Hepes (all from GibcoBRL; Rockville, Md.). Eighty percent confluent cells were washed twice with phosphate-buffered saline (GibcoBRL) and harvested using 0.25% trypsin (GibcoBRL). RNA was prepared using the RNeasy Maxi Kit from Qiagen (Valencia, Calif.).

cDNA template for real-time PCR may be polynucleotiderated using the Superscript™ First Strand Synthesis system for RT-PCR.

SYBR Green real-time PCR reactions were prepared as follows: The reaction mix consisted of 20 ng first strand cDNA; 50 nM Forward Primer; 50 nM Reverse Primer; 0.75×SYBR Green I (Sigma); 1×SYBR Green PCR Buffer (50 mM Tris-HCl pH8.3, 75 mM KCl); 10% DMSO; 3 mM MgCl₂; 300 μM each dATP, dGTP, dTTP, dCTP; 1 U Platinum® Taq DNA Polymerase High Fidelity (Cat#11304-029; Life Technologies; Rockville, Md.); 1:50 dilution; ROX (Life Technologies). Real-time PCR was performed using an Applied Biosystems 5700 Sequence Detection System. Conditions were 95° C. for 10 min (denaturation and activation of Platinum® Taq DNA Polymerase), 40 cycles of PCR (95° C. for 15 sec, 60° C. for 1 min). PCR products are analyzed for uniform melting using an analysis algorithm built into the 5700 Sequence Detection System.

| | |
|---|---|
| Forward primer: -F: | |
| 5′-GCTGGAGACCCTGATTTGCA-3′; | (SEQ ID NO:25) |
| and | |
| Reverse primer: -R: | |
| 5′-TGGACTTGATTGTGGCTTAGGTT-3′ | (SEQ ID NO:26) |

cDNA quantification used in the normalization of template quantity was performed using Taqman® technology. Taqman® reactions are prepared as follows: The reaction mix consisted of 20 ng first strand cDNA; 25 nM GAPDH-F3, Forward Primer; 250 nM GAPDH-R1 Reverse Primer; 200 nM GAPDH-PVIC Taqman® Probe (fluorescent dye labeled oligonucleotide primer); 1×Buffer A (Applied Biosystems); 5.5 mM MgCl2; 300 μM dATP, dGTP, dTTP, dCTP; 1 U Amplitaq Gold (Applied Biosystems). GAPDH, D-glyceraldehyde -3-phosphate dehydrogenase, was used as control to normalize mRNA levels.

Real-time PCR was performed using an Applied Biosystems 7700 Sequence Detection System. Conditions were 95° C. for 10 min. (denaturation and activation of Amplitaq Gold), 40 cycles of PCR (95° C. for 15 sec, 60° C. for 1 min).

The sequences for the GAPDH oligonucleotides used in the Taqman® reactions are as follows:

| | |
|---|---|
| GAPDH-F3- | |
| 5′-AGCCGAGCCACATCGCT-3′ | (SEQ ID NO:27) |
| GAPDH-R1- | |
| 5′-AGCCGAGCCACATCGCT-3′ | (SEQ ID NO:28) |
| GAPDH-PVIC Taqman® Probe -VIC- | |
| 5′-AGCCGAGCCACATCGCT-3′ TAMRA. | (SEQ ID NO:29) |

The Sequence Detection System polynucleotiderates a Ct (threshold cycle) value that is used to calculate a concentration for each input cDNA template. cDNA levels for each polynucleotide of interest are normalized to GAPDH cDNA levels to compensate for variations in total cDNA quantity in the input sample. This is done by polynucleotiderating GAPDH Ct values for each cell line. Ct values for the polynucleotide of interest and GAPDH are inserted into a modified version of the δδCt equation (Applied Biosystems Prism® 7700 Sequence Detection System User Bulletin #2) which is used to calculate a GAPDH normalized relative cDNA level for each specific cDNA. The δδCt equation is as follows: relative quantity of nucleic acid template=2^{δδCt}=2^{(δCta-δCtb)}, where δCta=Ct target—Ct GAPDH, and δCtb=Ct reference—Ct GAPDH. (No reference cell line was used for the calculation of relative quantity; δCtb was defined as 21).

The Graph Position of Table 1 corresponds to the tissue type position number of FIG. 9. Interestingly, IMRRP1 (also IMRRP1b) was found to be expressed greater in breast, colon, and lung carcinoma cell lines in comparison to other cancer cell lines in the OCLP-1 (oncology cell line panel). IMRRP1 is also expressed at moderate levels in prostate and ovarian cancer cell lines.

TABLE 1

| Graph | | | |
|---|---|---|---|
| position | Name | Tissue | Quant. |
| 1 | A-427 | lung | 2.2E+02 |
| 2 | A431 | squamous | 3.4E+02 |
| 3 | A2780/DDP-S | ovarian | 3.2E+02 |
| 4 | A2780/DDP-R | ovarian | 7.0E+01 |
| 5 | HCT116/epo5 | colon | 2.0E+02 |
| 6 | A2780/TAX-R | ovarian | 6.3E+02 |
| 7 | A2780/TAX-S | ovarian | 5.5E+02 |
| 8 | A549 | lung | 2.0E+02 |
| 9 | AIN4/myc | breast | 3.4E+02 |
| 10 | AIN 4T | breast | 3.7E+02 |
| 11 | AIN 4 | breast | 4.6E+02 |
| 12 | BT-549 | breast | 4.3E+02 |
| 13 | BT-20 | breast | 2.1E+02 |
| 14 | C-33A | cervical | 2.5E+02 |
| 15 | CACO-2 | colon | 2.5E+02 |
| 16 | Calu-3 | lung | 3.9E+02 |
| 17 | Calu-6 | lung | 3.7E+02 |
| 18 | BT-474 | breast | 2.0E+02 |
| 19 | Cx-1 | colon | 1.7E+02 |
| 20 | CCRF-CEM | leukemia | 2.6E+02 |
| 21 | ChaGo-K-1 | lung | 8.7E+02 |
| 22 | DU4475 | breast | 4.0E+02 |
| 23 | ES-2 | ovarian | 3.4E+02 |
| 24 | H3396 | breast | 1.1E+03 |
| 25 | HBL100 | breast | 2.8E+02 |
| 26 | HCT116/VM46 | colon | 3.4E+02 |
| 27 | HCT116/VP35 | colon | 1.9E+02 |
| 28 | HCT116 | colon | 2.9E+02 |
| 29 | A2780/epo5 | ovarian | 4.4E+02 |
| 30 | HCT116/ras | colon | 3.0E+02 |
| 31 | HCT116/TX15CR | colon | 4.2E+02 |
| 32 | HT-29 | colon | 3.1E+02 |
| 33 | HeLa | cervical | 3.5E+02 |
| 34 | Her2 MCF-7 | breast | 1.0E+03 |
| 35 | HL-60 | leukemia | 1.7E+02 |
| 36 | HOC-76 | ovarian | Mouse |
| 37 | Hs 294T | melanoma | 6.9E+02 |
| 38 | HS 578T | breast | 1.8E+02 |
| 39 | HT-1080 | fibrosarcoma | 2.6E+02 |
| 40 | HCT116/vivo | colon | 5.1E+02 |
| 41 | HT-3 | cervical | 6.1E+01 |
| 42 | K562 | leukemia | 2.2E+02 |
| 43 | SiHa | cervical | 1.5E+02 |
| 44 | LNCAP | prostate | 1.2E+02 |
| 45 | LS 174T | colon | 2.1E+02 |
| 46 | LX-1 | lung | 5.5E+02 |
| 47 | MCF7 | breast | 6.5E+02 |
| 48 | MCF-7/AdrR | breast | 3.5E+02 |
| 49 | MDA-MB-175-VII | breast | 1.2E+02 |
| 50 | MDA-MB-231 | breast | 3.4E+02 |
| 51 | MDA-MB-453 | breast | 9.3E+02 |
| 52 | MDA-MB-468 | breast | 7.3E+02 |
| 53 | MDAH 2774 | breast | 2.7E+02 |
| 54 | ME-180 | cervical | 3.5E+02 |
| 55 | MIP | colon | 3.6E+02 |
| 56 | ddH2O | colon | ND |
| 57 | SK-CO-1 | colon | 6.3E+02 |
| 58 | LoVo | colon | 5.2E+02 |
| 59 | SHP-77 | lung | 2.0E+03 |
| 60 | T84 | colon | 2.8E+02 |
| 61 | BT-483 | breast | 8.7E+02 |
| 62 | CCD-18Co | colon, fibroblast | 2.6E+02 |
| 63 | Colo320DM | colon | 2.9E+02 |
| 64 | DMS 114 | lung | 1.2E+03 |
| 65 | Sk-LU-1 | lung | 1.3E+02 |
| 66 | SK-MES-1 | lung | 3.7E+02 |
| 67 | SW1573 | lung | 4.4E+02 |
| 68 | SW 626 | ovarian | 4.5E+02 |
| 69 | SW1271 | lung | 6.4E+02 |
| 70 | SW756 | cervical | 2.0E+02 |
| 71 | SW900 | lung | 1.2E+03 |
| 72 | T47D | breast | 1.2E+03 |
| 73 | UACC-812 | breast | 5.5E+02 |
| 74 | UPN251 | ovarian | 6.1E+02 |
| 75 | ZR-75-1 | breast | 3.5E+02 |
| 76 | SKBR3 | breast | 3.7E+02 |
| 77 | SW403 | colon | 7.0E+02 |
| 78 | SW837 | colon | 7.4E+02 |
| 79 | CCD-112Co | colon | 6.6E+02 |
| 80 | Colo201 | colon | 8.4E+02 |
| 81 | PC-3 | prostate | 4.7E+02 |
| 82 | OVCAR-3 | ovarian | 2.8E+02 |
| 83 | SW480 | colon | 8.6E+02 |
| 84 | SW620 | colon | 6.5E+02 |
| 85 | SW1417 | colon | 5.1E+02 |
| 86 | Cob 205 | colon | 1.1E+03 |
| 87 | HCT-8 | colon | 8.2E+02 |
| 88 | PA-1 | ovarian | 5.5E+02 |
| 89 | CCD-33Co | colon | 4.5E+02 |
| 90 | MRC-5 | lung | 2.6E+02 |
| 91 | Pat-21 R60 | breast | ND |
| 92 | NCI-H596 | lung | 4.7E+02 |
| 93 | MSTO-211H | lung | 2.6E+02 |
| 94 | Caov-3 | ovarian | 1.4E+02 |
| 95 | Ca Ski | cervical | 4.3E+02 |
| 96 | LS123 | colon | 4.5E+02 |

### Example 12

Method of Assessing the Expression Profile of the Novel Imidazoline Receptor Polypeptides of the Present Invention Using Expanded mRNA Tissue and Cell Sources

Total RNA from tissues was isolated using the TriZol protocol (Invitrogen) and quantified by determining its absorbance at 260 nM. An assessment of the 18 s and 28 s ribosomal RNA bands was made by denaturing gel electrophoresis to determine RNA integrity.

The specific sequence to be measured was aligned with related polynucleotides found in GenBank to identity regions of significant sequence divergence to maximize primer and probe specificity. Gene-specific primers and probes were designed using the ABI primer express software to amplify small amplicons (150 base pairs or less) to maximize the likelihood that the primers function at 100% efficiency. All primer/probe sequences were searched against Public Genbank databases to ensure target specificity. Primers and probes were obtained from ABI.

For IMRRP, the primer probe sequences were as follows

| | |
|---|---|
| Forward Primer | |
| 5′-GGGCAGGGAATGCTTTCTC-3′ | (SEQ ID NO:30) |
| Reverse Primer | |
| 5′-AGGTGCGAGCTGCTTGGA-3′ | (SEQ ID NO:31) |
| TaqMan Probe | |
| 5′-ACTTCTGCCCACCTGTTTGAGGTGGA-3′ | (SEQ ID NO:32) |

### DNA Contamination

To access the level of contaminating genomic DNA in the RNA, the RNA was divided into 2 aliquots and one half was treated with Rnase-free Dnase (Invitrogen). Samples from both the Dnase-treated and non-treated were then subjected to reverse transcription reactions with (RT+) and without (RT−) the presence of reverse transcriptase. TaqMan assays were carried out with polynucleotide-specific primers (see above) and the contribution of genomic DNA to the signal detected was evaluated by comparing the threshold cycles obtained with the RT+/RT− non-Dnase treated RNA to that on the RT+/RT− Dnase treated RNA. The amount of signal contributed by genomic DNA in the Dnased RT-RNA must be less that 10% of that obtained with Dnased RT+RNA. If not the RNA was not used in actual experiments.

### Reverse Transcription Reaction and Sequence Detection

100 ng of Dnase-treated total RNA was annealed to 2.5 μM of the respective polynucleotide-specific reverse primer in the presence of 5.5 mM Magnesium Chloride by heating the sample to 72° C. for 2 min and then cooling to 55° C. for 30 min. 1.25 U/μl of MuLv reverse transcriptase and 500 μM of each dNTP was added to the reaction and the tube was incubated at 37° C. for 30 min. The sample was then heated to 90° C. for 5 min to denature enzyme.

Quantitative sequence detection was carried out on an ABI PRISM 7700 by adding to the reverse transcribed reaction 2.5 μM forward and reverse primers, 2.0 μM of the TaqMan probe, 500 μM of each dNTP, buffer and 5U AmpliTaq Gold™. The PCR reaction was then held at 94° C. for 12 min, followed by 40 cycles of 94° C. for 15 sec and 60° C. for 30 sec.

### Data Handling

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{(ΔCt)}.

The expanded expression profile of the IMRRP polypeptide is provided in FIG. 10 and FIG. 11 and described elsewhere herein.

### References

Altschul, S. F., Madden, T. L., Schaffer, A. A., Zhang, J., Zhang, Z., Miller, W., and Lipman, D. L. (1997). Gapped BLAST and PSI-BLAST: a new polynucleotideration of protein database search programs. Nucleic Acid Res. 25, 3389-3402.

Escriba, P. V., Ozaita, A., Miralles, A., Reis, D. J., and Garcia-Sevilla, J. A. (1995). Molecular characterization and isolation of a 45-kilodalton imidazoline receptor protein from the rat brain. Molec. Brain. Res. 32, 187-196.

Farsang, C., and Kapocsi, J. (1999). Imidazoline receptors: from discovery to antihypertensive therapy (facts and doubts). Brain Res. Bull. 49, 317-331.

Garcia-Sevilla, J. A., Escriba , P. V., and Guimon, J. (1999). Imidazoline receptors and human brain disorders. Ann. N. Y. Acad. Sci . 21, 392-409.

What is claimed is:

1. An isolated nucleic acid molecule consisting of a polynucleotide having a nucleotide sequence selected from the group consisting of:
(a) a polynucleotide fragment of SEQ ID NO:1 or a polynucleotide fragment of the cDNA sequence included in ATCC Deposit No:PTA-2671, which is hybridizable to SEQ ID NO:1;
(b) a polynucleotide encoding a polypeptide fragment of SEQ ID NO:3 or a polypeptide fragment encoded by the cDNA sequence included in ATCC Deposit No:PTA-2671, which is hybridizable to SEQ ID NO:1;
(c) a polynucleotide encoding a polypeptide domain of SEQ ID NO:3 or a polypeptide domain encoded by the cDNA sequence included in ATCC Deposit No:PTA-2671, which is hybridizable to SEQ ID NO:1;
(d) a polynucleotide encoding a polypeptide epitope of SEQ ID NO:3 or a polypeptide epitope encoded by the cDNA sequence included in ATCC Deposit No:PTA-267 1, which is hybridizable to SEQ ID NO:1;
(e) a polynucleotide encoding a polypeptide of SEQ ID NO:3 or the cDNA sequence included in ATCC Deposit No:PTA-2671, which is hybridizable to SEQ ID NO:1, having biological activity;
(f) an isolated polynucleotide comprising nucleotides 4 to 2472 of SEQ ID NO:1, wherein said nucleotides encode amino acids 2 to 824 of SEQ ID NO:3 minus the start methionine;
(g) an isolated polynucleotide comprising nucleotides 1 to 2472 of SEQ ID NO:1, wherein said nucleotides encode amino acids 1 to 824 of SEQ ID NO:3 including the start methionine;
(h) a polynucleotide which represents the complimentary sequence (antisense)of SEQ ID NO:1;
(i) a polynucleotide fragment of SEQ ID NO:2 or a polynucleotide fragment of the cDNA sequence included in ATCC Deposit No:PTA-2671, which is hybridizable to SEQ ID NO:2;
(j) a polynucleotide encoding a polypeptide fragment of SEQ ID NO:4 or a polypeptide fragment encoded by the cDNA sequence included in ATCC Deposit No:PTA-2671, which is hybridizable to SEQ ID NO:2;
(k) a polynucleotide encoding a polypeptide domain of SEQ ID NO:4 or a polypeptide domain encoded by the cDNA sequence included in ATCC Deposit No:PTA-2671, which is hybridizable to SEQ ID NO:2;
(l) a polynucleotide encoding a polypeptide epitope of SEQ ID NO:4 or a polypeptide epitope encoded by the cDNA sequence included in ATCC Deposit No:PTA-2671, which is hybridizable to SEQ ID NO:2;
(m) a polynucleotide encoding a polypeptide of SEQ ID NO:4 or the cDNA sequence included in ATCC Deposit No:PTA-2671, which is hybridizable to SEQ ID NO:2, having biological activity;
(n) an isolated polynucleotide comprising nucleotides 4 to 3297 of SEQ ID NO:1, wherein said nucleotides encode amino acids 2 to 1099 of SEQ ID NO:3 minus the start methionine;
(o) an isolated polynucleotide comprising nucleotides 1 to 3297 of SEQ ID NO:1, wherein said nucleotides encode amino acids 1 to 1099 of SEQ ID NO:3 including the start methionine;
(p) a polynucleotide which represents the complimentary sequence (antisense) of SEQ ID NO:2;
(q) a polynucleotide capable of hybridizing under stringent conditions to any one of the polynucleotides specified in (a)-(p) wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding an imidazoline receptor protein.

3. A recombinant vector comprising the isolated nucleic acid molecule of claim 1.

4. The recombinant host cell of claim 6 comprising vector sequences.

5. An isolated polypeptide comprising an amino acid sequence selected from the group consisting of:
(a) a polypeptide fragment of SEQ ID NO:3 or the encoded sequence included in ATCC Deposit No:PTA-2671;
(b) a polypeptide fragment of SEQ ID NO:3 or the encoded sequence included in ATCC Deposit No:PTA-2671, having biological activity;
(c) a polypeptide domain of SEQ ID NO:3 or the encoded sequence included in ATCC Deposit No:PTA-2671;
(d) a polypeptide epitope of SEQ ID NO:3 or the encoded sequence included in ATCC Deposit No:PTA-2671;
(e) a full length protein of SEQ ID NO:3 or the encoded sequence included in ATCC Deposit No:PTA-2671;
(f) comprising amino acids 2 to 337 of SEQ ID NO:3, wherein said amino acids 2 to 337 comprise a polypeptide of SEQ ID NO:3 minus the start methionine;
(g) a polypeptide comprising amino acids 1 to 337 of SEQ ID NO:3;
(h) a polypeptide fragment of SEQ ID NO:3 or the encoded sequence included in ATCC Deposit No:PTA-2671;
(i) a polypeptide fragment of SEQ ID NO:3 or the encoded sequence included in ATCC Deposit No:PTA-2671, having biological activity;
(j) a polypeptide domain of SEQ ID NO:3 or the encoded sequence included in ATCC Deposit No:PTA-2671;
(k) a polypeptide epitope of SEQ ID NO:3 or the encoded sequence included in ATCC Deposit No:PTA-2671;
(l) a full length protein of SEQ ID NO:3 or the encoded sequence included in ATCC Deposit No:PTA-2671;
(m) comprising amino acids 2 to 337 of SEQ ID NO:3, wherein said amino acids 2 to 337 comprise a polypeptide of SEQ ID NO:3 minus the start methionine; and
(n) a polypeptide comprising amino acids 1 to 337 of SEQ ID NO:3.

6. An isolated antibody that binds specifically to the isolated polypeptide of claim 8.

7. A recombinant host cell that expresses the isolated polypeptide of claim 8.

8. A method of making an isolated polypeptide comprising:
(a) culturing the recombinant host cell of claim 10 under conditions such that said polypeptide is expressed; and
(b) recovering said polypeptide.

9. A polypeptide produced by claim 11.

10. A method for preventing, treating, or ameliorating a medical condition, comprising administering to a mammalian subject a therapeutically effective amount of the polypeptide of claim 8 or a modulator thereof.

11. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

12. A method of diagnosing a pathological condition or a susceptibility to a pathological condition in a subject comprising:
(a) determining the presence or amount of expression of the polypeptide of claim 8 in a biological sample; and
(b) diagnosing a pathological condition or a susceptibility to a pathological condition based on the presence or amount of expression of the polypeptide.

13. The method of diagnosing a pathological condition of claim 15 wherein the condition is a member of the group consisting of: a disorder related to aberrant NF-kB activity; disorders related to aberrant IkBa expression or activity; a disorder linked to aberrant DNA synthesis; a disorder related to aberrant imidazoline receptor activity or expression; a disorder related to aberrant kinase activity; a disorder related to aberrant serine/threonine activity; proliferative disorder associated with p21 modulation; cellular proliferation in rapidly proliferating cells; disorders in which increased number of cells in the G1 phase of the cell cycle would be therapeutically beneficial; disorders in which decreased number of cells in the G1 phase of the cell cycle would be therapeutically beneficial; disorders in which increased number of cells in the G2 phase of the cell cycle would be therapeutically beneficial; disorders in which decreased number of cells in the G2 phase of the cell cycle would be therapeutically beneficial; disorders in which decreased number of cells that progress into the S phase of the cell cycle would be therapeutically beneficial; disorders in which increased number of cells that progress into the M phase of the cell cycle would be therapeutically beneficial; disorders in which decreased number of cells that progress into the M phase of the cell cycle would be therapeutically beneficial; disorders associated with aberrant p21 activity; disorders associated with aberrant p21 expression; disorders related to aberrant signal transduction; proliferative disorder of the colon; colon cancer; colon adenocarcinoma; Peutz-Jeghers polyposis; intestinal polyps; disorders associated with the immune response to tumors; proliferative disorder of the kidney; kidney tumors; other proliferative diseases and/or disorders; male reproductive system disorders; testicular disorders; spermatogenesis disorders; infertility; Klinefelter's syndrome; XX male; epididymitis; genital warts; germinal cell aplasia; cryptorchidism; varicocele; immotile cilia syndrome; viral orchitis; proliferative disorder of the testis; testicular cancer; choriocarcinoma; Nonseminoma; seminona; disorders of the breast; proliferative breast disorders; breast cancer; disorders of the lung; proliferative lung disorders; lung cancer; a disorder wherein increased NFkB expression or activity would be therapeutically beneficial; a disorder wherein decreased NFkB expression or activity would be therapeutically beneficial; a disorder wherein increased IkB expression or activity would be therapeutically beneficial; a disorder wherein decreased IkB expression or activity would be therapeutically beneficial; a disorder wherein increased apoptosis would be therapeutically beneficial; a disorder wherein decreased apoptosis would be therapeutically beneficial; healing disorder; necrosis disorder; aberrant regulation of blood pressure; feeding disorders; aberrant stimulation of locus coeruleus neurons; aberrant stimulation of insulin release; aberrant induction of the expression of glial fibrillary acidic protein independent of the action of alpha-2 adrenoceptors; dysphoric premenstrual syndrome; neurodegenerative disorders such as Alzheimer's disease; opiate addiction; monoamine turnover; nociception; aging; mood and stroke; salivary disorders and developmental disorders.

14. A method for treating, or ameliorating a medical condition with the polypeptide provided as SEQ ID NO:3, or a modulator thereof, wherein the medical condition is a member of the group consisting of: a disorder related to aberrant NF-kB activity; disorders related to aberrant IkBa expression or activity; a disorder linked to aberrant DNA synthesis; a disorder related to aberrant imidazoline receptor activity or expression; a disorder related to aberrant kinase activity; a disorder related to aberrant serine/threonine activity; proliferative disorder associated with p21 modulation; cellular proliferation in rapidly proliferating cells; disorders in which increased number of cells in the G1 phase of the cell cycle would be therapeutically beneficial; disorders in which decreased number of cells in the G1 phase of the cell cycle would be therapeutically beneficial; disorders in which increased number of cells in the G2 phase of the cell cycle would be therapeutically beneficial; disorders in which decreased number of cells in the G2 phase of the cell cycle would be therapeutically beneficial; disorders in which decreased number of cells that progress into the S phase of the cell cycle would be therapeutically beneficial; disorders in which increased number of cells that progress into the M phase of the cell cycle would be therapeutically beneficial; disorders in which decreased number of cells that progress into the M phase of the cell cycle would be therapeutically beneficial; disorders associated with aberrant p21 activity; disorders associated with aberrant p21 expression; disorders related to aberrant signal transduction; proliferative disorder of the colon; colon cancer; colon adenocarcinoma; Peutz-Jeghers polyposis; intestinal polyps; disorders associated with the immune response to tumors; proliferative disorder of the kidney; kidney tumors; other proliferative diseases and/or disorders; male reproductive system disorders; testicular disorders; spermatogenesis disorders; infertility; Klinefelter's syndrome; XX male; epididymitis; genital warts; germinal cell aplasia; cryptorchidism; varicocele; immotile cilia syndrome; viral orchitis; proliferative disorder of the testis; testicular cancer; choriocarcinoma; Nonseminoma; seminona; disorders of the breast; proliferative breast disorders; breast cancer; disorders of the lung; proliferative lung disorders; lung cancer; a disorder wherein increased NFkB expression or activity would be therapeutically beneficial; a disorder wherein decreased NFkB expression or activity would be therapeutically beneficial; a disorder wherein increased IkB expression or activity would be therapeutically beneficial; a disorder wherein decreased IkB expression or activity would be therapeutically beneficial; a disorder wherein increased apoptosis would be therapeutically beneficial; a disorder wherein decreased apoptosis would be therapeutically beneficial; healing disorder; necrosis disorder; aberrant regulation of blood pressure; feeding disorders; aberrant stimulation of locus coeruleus neurons; aberrant stimulation of insulin release; aberrant induction of the expression of glial fibrillary acidic protein independent of the action of alpha-2 adrenoceptors; dysphoric premenstrual syndrome; neurodegenerative disorders such as Alzheimer's disease; opiate addiction; monoamine turnover; nociception; aging; mood and stroke; salivary disorders and developmental disorders.

15. A method for treating, or ameliorating a medical condition according to claim 14 wherein the modulator is a member of the group consisting of: a small molecule, a peptide, and an antisense molecule.

16. A method for treating, or ameliorating a medical condition according to claim 15 wherein the modulator is an antagonist.

17. A method for treating, or ameliorating a medical condition according to claim 15 wherein the modulator is an agonist.

18. A method of screening for candidate compounds capable of modulating the activity of a receptor polypeptide, comprising:
(a) contacting a test compound with a cell or tissue expressing the polypeptide comprising an amino acid sequence as set forth in SEQ ID NO:3 or SEQ ID NO:4; and
(b) selecting as candidate modulating compounds those test compounds that modulate activity of the receptor polypeptide.


SEQUENCE LISTING (34 sequences)

SEQ ID NO: 1 - DNA, 2475 bp, Homo sapiens
atgttcggctccgccccccagcgtcccgtggccatgacgaccgctcagagggactccctgttgtggaagctcgcggggttgctgcgggagtccggggatgtggtcctgtctggctgtagcaccctgagcctgctgactcccacactgcaacagctgaaccacgtatttgagctgcacctggggccatggggccctggccagacaggctttgtggctctgccctcccatcctgccgactcccctgttattcttcagcttcagtttctcttcgatgtgctgcagaaaacactttcactcaagctggtccatgttgctggtcctggccccacagggcccatcaagattttccccttcaaatcccttcggcacctggagctccgaggtgttcccctccactgtctgcatggcctccgaggcatctactcccagctggagaccctgatttgcagcaggagcctccaggcattagaggagctcctctcagcctgcggcggcgacttctgctctgccctcccttggctggctctgctttctgccaacttcagctacaatgcactgaccgccttagacagctccctgcgcctcttgtcagctctgcgtttcttgaacctaagccacaatcaagtccaggactgtcagggattcctgatggatttgtgtgagctccaccatctggacatctcctataatcgcctgcatttggtgccaagaatgggaccctcaggggctgctctgggggtcctgatactgcgaggcaatgagcttcggagcctgcatggcctagagcagctgaggaatctgcggcacctggatttggcatacaacctgctggaaggacaccgggagctgtcaccactgtggctgctggctgagctccgcaagctctacctggaggggaaccctctttggttccaccctgagcaccgagcagccactgcccagtacttgtcaccccgggccagggatgctgctactggcttccttctcgatggcaaggtcttgtcactgacagattttcagactcacacatccttggggctcagccccatgggcccacctttgccctggccagtggggagtactcctgaaacctcaggtggccctgacctgagtgacagcctctcctcagggggtgttgtgacccagcccctgcttcataaggttaagagccgagtccgtgtgaggcgggcaagcatctctgaacccagtgatacggacccggagccccgaactctgaacccctctccggctggatggttcgtgcagcagcacccggagctggagctcatgagcagcttccgggaacggttcggccgcaactggctgcagtacaggagtcacctggagccctccggaaaccctctgccggccacccccactacttctgcacccagtgcacctccagccagctcccagggccccgacactgcacccagaccttcacccccgcaggaggaagccagaggcccccaggagtcaccacagaaaatgtcagaggaggtcagggcggagccacaggaggaggaagaggagaaggaggggaaggaggagaaggaggagggggagatggtggaacagggagaagaggaggcaggagaggaggaagaagaggagcaggaccagaaggaagtggaagcggaactctgtcgccccttgttggtgtgtcccctggaggggcctgagggcatacggggcagggaatgctttctcagggtcacttctgcccacctgtttgaggtggaactccaagcagctcgcaccttggagcgactggagctccagagtctggaggcagctgagatagagccggaggcccaggcccagaggtcgcccaggcccacgggctcagatctgctccctggagcccccatcctcagtctgcgcttctcctacatctgccctgaccggcagttgcgtcgctatttggtgctggagcctgatgcccacgcagctgtccaggagctgcttgccgtgttgaccccagtcaccaatgtggctcgggaacagcttggggaggccagggacctcctgctgggtagattccagtgtctacgctgtggccatgagttcaagccagaggagcccaggatgggattagacagtgaggaaggctggaggcctctgttccaaaagacagggagcggaaacagggagagcagtctctggctccttctccgtttgccagccctgtctgccaccctcctggccatggtgaccaccttgacagggccaagaacagcccacctcaggcaccgagcacccgtgaccatggtagttggagcctcagtcccccccctgagcgctgtggcctccgctctgtggaccaccgactccggctcttcctggatgttgaggtgttcagcgatgcccaggaggagttccagtgctgcctcaaggtgccagtggcattggcaggccacactggggagttcatgtgccttgtggttgtgtctgaccgcaggctgtacctgttga

SEQ ID NO: 2 - DNA, 3300 bp, Homo sapiens
atgttcggctccgccccccagcgtcccgtggccatgacgaccgctcagagggactccctgttgtggaagctcgcggggttgctgcgggagtccggggatgtggtcctgtctggctgtagcaccctgagcctgctgactcccacactgcaacagctgaaccacgtatttgagctgcacctggggccatggggccctggccagacaggctttgtggctctgccctcccatcctgccgactcccctgttattcttcagcttcagtttctcttcgatgtgctgcagaaaacactttcactcaagctggtccatgttgctggtcctggccccacagggcccatcaagattttccccttcaaatcccttcggcacctggagctccgaggtgttcccctccactgtctgcatggcctccgaggcatctactcccagctggagaccctgatttgcagcaggagcctccaggcattagaggagctcctctcagcctgcggcggcgacttctgctctgccctcccttggctggctctgctttctgccaacttcagctacaatgcactgaccgccttagacagctccctgcgcctcttgtcagctctgcgttcttgaacctaagccacaatcaagtccaggactgtcagggattcctgatggatttgtgtgagctccaccatctggacatctcctataatcgcctgcatttggtgccaagaatgggaccctcaggggctgctctgggggtcctgatactgcgaggcaatgagcttcggagcctgcatggcctagagcagctgaggaatctgcggcacctggatttggcatacaacctgctggaaggacaccgggagctgtcaccactgtggctgctggctgagctccgcaagctctacctggaggggaaccctctttggttccaccctgagcaccgagcagccactgcccagtacttgtcaccccgggccagggatgctgctactggcttccttctcgatggcaaggtcttgtcactgacagattttcagactcacacatccttggggctcagccccatgggcccacctttgccctggccagtggggagtactcctgaaacctcaggtggccctgacctgagtgacagcctctcctcagggggtgttgtgacccagcccctgcttcataaggttaagagccgagtccgtgtgaggcgggcaagcatctctgaacccagtgatacggacccggagccccgaactctgaacccctctccggctggatggttcgtgcagcagcacccggagctggagctcatgagcagcttccgggaacggttcggccgcaactggctgcagtacaggagtcacctggagccctccggaaaccctctgccggccacccccactacttctgcacccagtgcacctccagccagctcccagggccccgacactgcacccagaccttcacccccgcaggaggaagccagaggcccccaggagtcaccacagaaaatgtcagaggaggtcagggcggagccacaggaggaggaagaggagaaggaggggaaggaggagaaggaggagggggagatggtggaacagggagaagaggaggcaggagaggaggaagaagaggagcaggaccagaaggaagtggaagcggaactctgtcgccccttgttggtgtgtcccctggaggggcctgagggcgtacggggcagggaatgctttctcagggtcacttctgcccacctgtttgaggtggaactccaagcagctcgcaccttggagcgactggagctccagagtctggaggcagctgagatagagccggaggcccaggcccagaggtcgcccaggcccacgggctcagatctgctccctggagcccccatcctcagtctgcgcttctcctacatctgccctgaccggcagttgcgtcgctatttggtgctggagcctgatgcccacgcagctgtccaggagctgcttgccgtgttgaccccagtcaccaatgtggctcgggaacagcttggggaggccagggacctcctgctgggtagattccagtgtctacgctgtggccatgagttcaagccagaggagcccaggatgggattagacagtgaggaaggctggaggcctctgttccaaaagacagaatctcctgctgtgtgtcctaactgtggtagtgaccacgtggttctcctcgctgtgtctcggggaacccccaacagggagcggaaacagggagagcagtctctggctccttctccgtttgccagccctgtctgccaccctcctggccatggtgaccaccttgacagggccaagaacagcccacctcaggcaccgagcacccgtgaccatggtagttggagcctcagtcccccccctgagcgctgtggcctccgctctgtggaccaccgactccggctcttcctggatgttgaggtgttcagcgatgcccaggaggagttccagtgctgcctcaaggtgccagtggcattggcaggccacactggggagttcatgtgccttgtggttgtgtctgaccgcaggctgtacctgttgaaggtgactggggagatgcgtgagcctccagctagctggctgcagctgaccctggctgttcccctgcaggatctgagtggcatagagctgggcctggcaggccagagcctgcggctagagtgggcagctggggcgggccgctgtgtgctgctgccccgagatgccaggcattgccgggccttcctagaggagctccttgatgtcttgcagtctctgccccctgcctggaggaactgtgtcagtgccacagaggaggaggtcaccccccagcaccggctctggccattgctggaaaaagactcatccttggaggctcgccagttcttctaccttcgggcgttcctggttgaaggcccttccacctgcctcgtatccctgttgctgactccgtccaccctgttcctgttagatgaggatgctgcagggtccccggcagagccctctcctccagcagcatctggcgaagcctctgagaaggtgcctccctcggggccgggccctgctgtgcgtgtcagggagcagcagccactcagcagcctgagctccgtgctgctctaccgctcagcccctgaggacttgcggctgctcttctacgatgaggtgtcccggctggagagcttttgggcactccgtgtggtgtgtcaggagcagctgacagccctgcttgcctggatccgggaaccatgggaggagctgttttccatcggactccggacagtgatccaagaggcgctggcccttgaccgatgav

SEQ ID NO: 3 - protein, 824 aa, Homo sapiens
MFGSAPQRPVAMTTAQRDSLLWKLAGLLRESGDVVLSGCSTLSLLTPTLQQLNHVFELHLGPWGPGQTGFVALPSHPADSPVILQLQFLFDVLQKTLSLKLVHVAGPGPTGPIKIFPFKSLRHLELRGVPLHCLHGLRGIYSQLETLICSRSLQALEELLSACGGDFCSALPWLALLSANFSYNALTALDSSLRLLSALRFLNLSHNQVQDCQGFLMDLCELHHLDISYNRLHLVPRMGPSGAALGVLILRGNELRSLHGLEQLRNLRHLDLAYNLLEGHRELSPLWLLAELRKLYLEGNPLWFHPEHRAATAQYLSPRARDAATGFLLDGKVLSLTDFQTHTSLGLSPMGPPLPWPVGSTPETSGGPDLSDSLSSGGVVTQPLLHKVKSRVRVRRASISEPSDTDPEPRTLNPSPAGWFVQQHPELELMSSFRERFGRNWLQYRSHLEPSGNPLPATPTTSAPSAPPASSQGPDTAPRPSPPQEEARGPQESPQKMSEEVRAEPQEEEEEKEGKEEKEEGEMVEQGEEEAGEEEEEEQDQKEVEAELCRPLLVCPLEGPEGIRGRECFLRVTSAHLFEVELQAARTLERLELQSLEAAEIEPEAQAQRSPRPTGSDLLPGAPILSLRFSYICPDRQLRRYLVLEPDAHAAVQELLAVLTPVTNVAREQLGEARDLLLGRFQCLRCGHEFKPEEPRMGLDSEEGWRPLFQKTGSGNRESSLWLLLRLPALSATLLAMVTTLTGPRTAHLRHRAPVTMVVGASVPPLSAVASALWTTDSGSSWMLRCSAMPRRSSSAASRCQWHWQATLGSSCALWLCLTAGCTC

SEQ ID NO: 4 - protein, 1099 aa, Homo sapiens [sequence omitted]

SEQ ID NO: 5 - DNA, 20 bp, Homo sapiens
gctggagaccctgatttgca

SEQ ID NO: 6 - DNA, 23 bp, Homo sapiens
tggacttgattgtggcttaggtt

SEQ ID NO: 7 - protein, 1504 aa, Homo sapiens
MATARTFGPEREAEPAKEARVVGSELVDTYTVYIIQVTDGSHEWTVKHRYSDFHDLHEKLVAERKIDKNLLPPKKIIGKNSRSLVEKREKDLEVYLQKLLAAFPGVTPRVLAHFLHFHFYEINGITAALAEELFEKGEQLLGAGEVFAIGPLQLYAVTEQLQQGKPTCASGDAKTDLGHILDFTCRLKYLKVSGTEGPFGTSNIQEQLLPFDLSIFKSLHQVEISHCDAKHIRGLVASKPTLATLSVRFSATSMKEVLVPEASEFDEWEPEGTTLEGPVTAVIPTWQALTTLDLSHNSISEIDESVKLIPKIEFLDLSHNGLLVVDNLQHLYNLVHLDLSYNKLSSLEGLHTKLGNIKTLNLAGNLLESLSGLHKLYSLVNLDLRDNRIEQMEEVRSIGSLPCLEHVSLLNNPLSIIPDYRTKVLAQFGERASEVCLDDTVTTEKELDTVEVLKAIQKAKEVKSKLSNPEKKGGEDSRLSAAPCIRPSSSPPTVAPASASLPQPILSNQGIMFVQEEALASSLSSTDSLTPEHQPIAQGCSDSLESIPAGQAASDDLRDVPGAVGGASPEHAEPEVQVVPGSGQIIFLPFTCIGYTATNQDFIQRLSTLIRQAIERQLPAWIEAANQREEGQGEQGEEEDEEEEEEEDVAENRYFEMGPPDVEEEEGGGQGEEEEEEEEDEEAEEERLALEWALGADEDFLLEHIRILKVLWCFLIHVQGSIRQFAACLVLTDFGIAVFEIPHQESRGSSQHILSSLRFVFCFPHGDLTEFGFLMPELCLVLKVRHSENTLFIISDAANLHEFHADLRSCFAPQHMAMLCSPILYGSHTSLQEFLRQLLTFYKVAGGCQERSQGCFPVYLVYSDKRMVQTAAGDYSGNIEWASCTLCSAVRRSCCAPSEAVKSAAIPYWLLLTPQHLNVIKADFNPMPNRGTHNCRNRNSFKLSRVPLSTVLLDPTRSCTQPRGAFADGHVLELLVGYRFVTAIFVLPHEKFHFLRVYNQLRASLQDLKTVVIAKTPGTGGSPQGSFADGQPAERRASNDQRPQEVPAEALAPAPVEVPAPAPAAASASGPAKTPAPAEASTSALVPEETPVEAPAPPPAEAPAQYPSEHLIQATSEENQIPSHLPACPSLRHVASLRGSAIIELFHSSIAEVENEELRHLMWSSVVFYQTPGLEVTACVLLSTKAVYFVLHDGLRRYFSEPLQDFWHQKNTDYNNSPFHISQCFVLKLSDLQSVNVGLFDQHFRLTGSTPMQVVTCLTRDSYLTHCFLQHLMVVLSSLERTPSPEPVDKDFYSEFGNKTTGKMENYELIHSSRVKFTYPSEEEIGDLTFTVAQKMAEPEKAPALSILLYVQAFQVGMPPPGCCRGPLRPKTLLLTSSEIFLLDEDCVHYPLPEFAKEPPQRDRYRLDDGRRVRDLDRVLMGYQTYPQALTLVFDDVQGHDLMGSVTLDHFGEVPGGPARASQGREVQWQVFVPSAESREKLISLLARQWEALCGRELPVELTG

SEQ ID NO: 8 - DNA, 100 bp, Homo sapiens
tacgctgtggccatgagttcaagccagaggagcccaggatgggattagacagtgaggaaggctggaggcctctgttccaaaagacagaatctcctgctgt

SEQ ID NO: 9 - DNA, 100 bp, Homo sapiens
gaacccccaacagggagcggaaacagggagagcagtctctggctccttctccgtttgccagccctgtctgccaccctcctggccatggtgaccaccttga

SEQ ID NO: 10 - DNA, 103 bp, Homo sapiens
tcatccttggaggctcgccagttcttctaccttcgggcgttcctggttgaaggcccttccacctgcctcgtatccctgttgctgactccgtccaccctgttcc

SEQ ID NO: 11 - protein, 1289 aa, Drosophila melanogaster
MDPQKITELANLLRQNGDKILSSEFTLTLSGSLLRALNDSFTLIADTEIGTGAGYLQPQSFQVVKPINAKSSVFPDLQLVHDFVQKTTLLKLTYFPSEHYFEGAIDIAKFRALRRLEVNKINIGQVVGIQPLRGQLQHLICVKSLTSVDDIITRCGGDNSNGFVWNELKTADFSYNSLRSVDTALEFAQHLQHLNLRHNKLTSVAAIKWLPHLKTLDLSYNCLTHLPQFHMEACKRLQLLNISNNYVEELLDVAKLDALYNLDLSDNCLLEHSQLLPLSALMSLIVLNLQGNPLACNPKHRQATAQYLHKNSATVKFVLDFEPLTKAEKALTGSQKWRYISGLSHRSPRSTSMSINSSSASINTSDGSQFSSFGSQRSVSIRGKNYTLEDNQSMDTSQSSKRISSCKIRTVDIEESSEINTDAASVSTPNPRSEYEEEPDNSHLETKKKIETLRLTYGNEWLKSGNAELMLGIETPQPTERERNESRQLFNEYLGELSGFTEAKNDSEHHNISSTPTNNVLLASTFDATITPIKSEANDTSGQTLYETCTEGEETNYESFGNNTTELSTEERPPDRHEELLRLYASSSNAQDEDPVSDAESDEETYIVYHEQKPSEVLFLTISSNFIREKDTLTERTKAKWSLKILESCERVRSNTLRINFDTMRKDKQERIYCVENTLCQELEKKLRDILSQRDLTEMNISIYRCVNCLTQFTIEQKSKRYKAKELRCPDCRSVYVAEVTELSSSLSKPSGEVAAEPKLSPAMIVEESPVEELAAAINKEESNSIGKSLASFLFYFDESSFDSNQSVVGSSNTDRDMEFRANESDVDIISNPSQSSIEVLDPNYVQSASRKTSEERRISQLPHLETIHDEVAKSKSFIEREFGQLLAEQAQPTTPSTAAPLAPAKSAVPSHVPLTESSSSGSVTDSICTTYEQQATDAPQNLQNSLLTESSNSQVSGSDAESNSRLKSAEDASLLPFASVFQSTNLLMSSSKKLIESEATVFGTQPYKFNYSDFNDIDHRLKLYFYQRKFKEDGEHFKWLAKGRIYNEQTQSLGEGLVVMSNCKCYLMEAFAEPHDDVAKWLRQVVSVAVNRLVAIDLLPWKLGLSFTLKDWGGFVLLLHDMLRTESLLNYLQQIPLPEQCKLNHQPSVTLSHQWETIASEPVKMCSLIPSCQWICDQEKSSFEPSLLLITETHLYISGNGKFSWLSDKVQEKPIQPELSLNQPLSNLVDVERITDQKYAINFIDETQNRCEIWKLQFETHANAACCLNVIGKGWEQLFGVPFSLSGT

SEQ ID NO: 12 - DNA, 20 bp, Homo sapiens
gctggagaccctgatttgca

SEQ ID NO: 13 - DNA, 23 bp, Homo sapiens
tggacttgattgtggcttaggtt

SEQ ID NO: 14 - DNA, 25 bp, Artificial Sequence
  Synthesized Oligonucleotide.
cccaggugcagcucaaauacguggu

SEQ ID NO: 15 - DNA, 25 bp, Artificial Sequence
  Synthesized Oligonucleotide.
cauucuuggcaccaaaugcaggcga

SEQ ID NO: 16 - DNA, 25 bp, Artificial Sequence
  Synthesized Oligonucleotide.
auuccucagcugcucuaggccaugc

SEQ ID NO: 17 - DNA, 25 bp, Artificial Sequence
  Synthesized Oligonucleotide.
guccuuccagcagguuguaugccaa

SEQ ID NO: 18 - DNA, 25 bp, Artificial Sequence
  Synthesized Oligonucleotide.
ccuugccaucgagaaggaagccagu

SEQ ID NO: 19 - DNA, 21 bp, Homo sapiens
ctggagactctcagggtcgaa

SEQ ID NO: 20 - DNA, 17 bp, Homo sapiens
gcgcttccaggactgca

SEQ ID NO: 21 - DNA, 27 bp, Homo sapiens
acagatttctaccactccaaacgccgg

SEQ ID NO: 22 - DNA, 24 bp, Homo sapiens
gaggatgaggagagctatgacaca

SEQ ID NO: 23 - DNA, 22 bp, Homo sapiens
ccctttgcactcataacgtcag

SEQ ID NO: 24 - DNA, 29 bp, Homo sapiens
aaacacacagtcatcatagggcagctcgt

SEQ ID NO: 25 - DNA, 20 bp, Homo sapiens
gctggagaccctgatttgca

SEQ ID NO: 26 - DNA, 23 bp, Homo sapiens
tggacttgattgtggcttaggtt

SEQ ID NO: 27 - DNA, 17 bp, Homo sapiens
agccgagccacatcgct

SEQ ID NO: 28 - DNA, 17 bp, Homo sapiens
agccgagccacatcgct

SEQ ID NO: 29 - DNA, 17 bp, Homo sapiens
agccgagccacatcgct

SEQ ID NO: 30 - DNA, 19 bp, Homo sapiens
gggcagggaatgctttctc

SEQ ID NO: 31 - DNA, 18 bp, Homo sapiens
aggtgcgagctgcttgga

SEQ ID NO: 32 - DNA, 26 bp, Homo sapiens
acttctgcccacctgtttgaggtgga

SEQ ID NO: 33 - protein, 1099 aa, Homo sapiens [sequence omitted]

SEQ ID NO: 34 - protein, 1099 aa, Homo sapiens [sequence omitted]